SEQUENCE LISTING (66 sequences)

SEQ ID NO: 1 - DNA, 42 bp, Artificial Sequence
  Description of Artificial Sequence Primer
ggctagagaattccaggtaagatgggcgatcccctcacctgg

SEQ ID NO: 2 - DNA, 22 bp, Artificial Sequence
  Description of Artificial Sequence Primer
ttgggtactcctcgctaggttc

SEQ ID NO: 3 - DNA, 21 bp, Artificial Sequence
  Description of Artificial Sequence Synthetic oligonucleotide
tctagagaattcgccaccatg

SEQ ID NO: 4 - RNA, 18 nt, Artificial Sequence
  Description of Artificial Sequence Synthetic oligonucleotide
ugaacccggggcggccgc

SEQ ID NO: 5 - DNA, 42 bp, Artificial Sequence
  Description of Artificial Sequence Primer
cagtacccgcgggccaccatgtttgactgtatggatgttctg

SEQ ID NO: 6 - DNA, 37 bp, Artificial Sequence
  Description of Artificial Sequence Primer
cagtacctgcagatcattgcacgagtggtgactgact

SEQ ID NO: 7 - DNA, 38 bp, Artificial Sequence
  Description of Artificial Sequence Primer
caggttattctagagtcgacgctctcattacttgtaac

SEQ ID NO: 8 - DNA, 41 bp, Artificial Sequence
  Description of Artificial Sequence Primer
cgaatgcgttctagagtcgaccatgttcaccagggattttg

SEQ ID NO: 9 - DNA, 23 bp, Artificial Sequence
  Description of Artificial Sequence Primer
cacctagcaggcgtgaccggtgg

SEQ ID NO: 10 - DNA, 37 bp, Artificial Sequence
  Description of Artificial Sequence Primer
cctaccaattgtataaaacccctcataaaaaccccac

SEQ ID NO: 11 - DNA, 24 bp, Artificial Sequence
  Description of Artificial Sequence Primer
cacaggtcaaacctcctaggaatg

SEQ ID NO: 12 - DNA, 21 bp, Artificial Sequence
  Description of Artificial Sequence Primer
tcctgctcaacttcctgtcga

SEQ ID NO: 13 - DNA, 19 bp, Artificial Sequence
  Description of Artificial Sequence Probe
cgagacgctaccatggcta

SEQ ID NO: 14 - DNA, 21 bp, Artificial Sequence
  Description of Artificial Sequence Primer
attgggagaccctttgacatt

SEQ ID NO: 15 - DNA, 30 bp, Artificial Sequence
  Description of Artificial Sequence Probe
caccttctctaacttcttgagcgccttgct

SEQ ID NO: 16 - DNA, 42 bp, Artificial Sequence
  Description of Artificial Sequence Primer
actgccgcgggccaccatgtttgactgtatggatgttctgtc

SEQ ID NO: 17 - DNA, 66 bp, Artificial Sequence
  Description of Artificial Sequence Primer
actgccgcgggccaccatggactacaaggacgacgatgacaagtttgactgtatggatgttctgtc

SEQ ID NO: 18 - DNA, 29 bp, Artificial Sequence
  Description of Artificial Sequence Primer
actggcggccgctcactgcagcagtggtg

SEQ ID NO: 19 - DNA, 38 bp, Artificial Sequence; identical to SEQ ID NO: 7
  Description of Artificial Sequence Primer

SEQ ID NO: 20 - DNA, 41 bp, Artificial Sequence; identical to SEQ ID NO: 8
  Description of Artificial Sequence Primer

SEQ ID NO: 21 - DNA, 10998 bp, Artificial Sequence
  Description of Artificial Sequence pONY8.0Z vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 22 - DNA, 12481 bp, Artificial Sequence
  Description of Artificial Sequence pONY3.1, EIAV gag/pol expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgaggtcgacggtatcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatccaatcatgatagatggggctggaaacagaaattttagacctctaacacctagaggatatactacttgggtgaataccatacagacaaatggtctattaaatgaagctagtcaaaacttatttgggatattatcagtagactgtacttctgaagaaatgaatgcatttttggatgtggtacctggccaggcaggacaaaagcagatattacttgatgcaattgataagatagcagatgattgggataatagacatccattaccgaatgctccactggtggcaccaccacaagggcctattcccatgacagcaaggtttattagaggtttaggagtacctagagaaagacagatggagcctgcttttgatcagtttaggcagacatatagacaatggataatagaagccatgtcagaaggcatcaaagtgatgattggaaaacctaaagctcaaaatattaggcaaggagctaaggaaccttacccagaatttgtagacagactattatcccaaataaaaagtgagggacatccacaagagatttcaaaattcttgactgatacactgactattcagaacgcaaatgaggaatgtagaaatgctatgagacatttaagaccagaggatacattagaagagaaaatgtatgcttgcagagacattggaactacaaaacaaaagatgatgttattggcaaaagcacttcagactggtcttgcgggcccatttaaaggtggagccttgaaaggagggccactaaaggcagcacaaacatgttataactgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatataatctagagaaaaggcctactacaatagtattaattaatgatactcccttaaatgtactgttagacacaggagcagatacttcagtgttgactactgcacattataataggttaaaatatagagggagaaaatatcaagggacgggaataataggagtgggaggaaatgtggaaacattttctacgcctgtgactataaagaaaaagggtagacacattaagacaagaatgctagtggcagatattccagtgactattttgggacgagatattcttcaggacttaggtgcaaaattggttttggcacagctctccaaggaaataaaatttagaaaaatagagttaaaagagggcacaatggggccaaaaattcctcaatggccactcactaaggagaaactagaaggggccaaagagatagtccaaagactattgtcagagggaaaaatatcagaagctagtgacaataatccttataattcacccatatttgtaataaaaaagaggtctggcaaatggaggttattacaagatctgagagaattaaacaaaacagtacaagtaggaacggaaatatccagaggattgcctcacccgggaggattaattaaatgtaaacacatgactgtattagatattggagatgcatatttcactatacccttagatccagagtttagaccatatacagctttcactattccctccattaatcatcaagaaccagataaaagatatgtgtggaaatgtttaccacaaggattcgtgttgagcccatatatatatcagaaaacattacaggaaattttacaaccttttagggaaagatatcctgaagtacaattgtatcaatatatggatgatttgttcatgggaagtaatggttctaaaaaacaacacaaagagttaatcatagaattaagggcgatcttactggaaaagggttttgagacaccagatgataaattacaagaagtgccaccttatagctggctaggttatcaactttgtcctgaaaattggaaagtacaaaaaatgcaattagacatggtaaagaatccaacccttaatgatgtgcaaaaattaatggggaatataacatggatgagctcagggatcccagggttgacagtaaaacacattgcagctactactaagggatgtttagagttgaatcaaaaagtaatttggacggaagaggcacaaaaagagttagaagaaaataatgagaagattaaaaatgctcaagggttacaatattataatccagaagaagaaatgttatgtgaggttgaaattacaaaaaattatgaggcaacttatgttataaaacaatcacaaggaatcctatgggcaggtaaaaagattatgaaggctaataagggatggtcaacagtaaaaaatttaatgttattgttgcaacatgtggcaacagaaagtattactagagtaggaaaatgtccaacgtttaaggtaccatttaccaaagagcaagtaatgtgggaaatgcaaaaaggatggtattattcttggctcccagaaatagtatatacacatcaagtagttcatgatgattggagaatgaaattggtagaagaacctacatcaggaataacaatatacactgatgggggaaaacaaaatggagaaggaatagcagcttatgtgaccagtaatgggagaactaaacagaaaaggttaggacctgtcactcatcaagttgctgaaagaatggcaatacaaatggcattagaggataccagagataaacaagtaaatatagtaactgatagttattattgttggaaaaatattacagaaggattaggtttagaaggaccacaaagtccttggtggcctataatacaaaatatacgagaaaaagagatagtttattttgcttgggtacctggtcacaaagggatatatggtaatcaattggcagatgaagccgcaaaaataaaagaagaaatcatgctagcataccaaggcacacaaattaaagagaaaagagatgaagatgcagggtttgacttatgtgttccttatgacatcatgatacctgtatctgacacaaaaatcatacccacagatgtaaaaattcaagttcctcctaatagctttggatgggtcactgggaaatcatcaatggcaaaacaggggttattaattaatggaggaataattgatgaaggatatacaggagaaatacaagtgatatgtactaatattggaaaaagtaatattaaattaatagagggacaaaaatttgcacaattaattatactacagcatcactcaaattccagacagccttgggatgaaaataaaatatctcagagaggggataaaggatttggaagtacaggagtattctgggtagaaaatattcaggaagcacaagatgaacatgagaattggcatacatcaccaaagatattggcaagaaattataagataccattgactgtagcaaaacagataactcaagaatgtcctcattgcactaagcaaggatcaggacctgcaggttgtgtcatgagatctcctaatcattggcaggcagattgcacacatttggacaataagataatattgacttttgtagagtcaaattcaggatacatacatgctacattattgtcaaaagaaaatgcattatgtacttcattggctattttagaatgggcaagattgttttcaccaaagtccttacacacagataacggcactaattttgtggcagaaccagttgtaaatttgttgaagttcctaaagatagcacataccacaggaataccatatcatccagaaagtcagggtattgtagaaagggcaaataggaccttgaaagagaagattcaaagtcatagagacaacactcaaacactggaggcagctttacaacttgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 23 - DNA, 29 bp, Artificial Sequence
  Description of Artificial Sequence Primer
accagtagttaatttctgagacccttgta

SEQ ID NO: 24 - DNA, 10112 bp, Artificial Sequence
  Description of Artificial Sequence pESYNGP, codon-optimised EIAV gag/pol expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattcgccaccatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 25 - DNA, 10114 bp, Artificial Sequence
  Description of Artificial Sequence pESDSYNGP, codon-optimised EIAV gag/pol expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattccaggtaagatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 26 - DNA, 6845 bp, Artificial Sequence
  Description of Artificial Sequence pRV67, VSV-G expression plasmid
tcgacctgcaggatatcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtaccgagctcgcgtaatcatgtcatagctgtttcctgtgtgaaattgttatccgctcacaattccacacaacatacgagccggaagcataaagtgtaaagcctggggtgcctaatgagtgagctaactacattaattgcgttgcgctcackgcccgctttccartcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacmctagaagracagtatttggkatctgsgcttctgytgaagmcagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaataagcggccgcggccatgccggccactagtctcgagttattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgaagacgtcgcctcctcactacttctggaatagctcagaggccgaggcggcctcggcctctgcataaataaaaaaaatwaktcasggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccatcgtgtcaaaggacagtgactgcagtgaataataaaatgtgtgtttgtccgaaatacgcgttttgagawttctgtcgccgactaaattcatgtcgcgcgatartggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggsggagyttctacatccgrrsccttgytcccatgcctccasgrmttcatgktcgytcggcagctccttgctcctaamcagtggaggccagacttaggcacagcacgatgcccaccaccaccagtgtgccgcacaaggccgtggcggtagggtatgtgtctgaaaatgagctcggggagcgggcttgcaccgctggacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttcccgggataggtacctcgcgagatccctcgaggaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatgggggcctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagggatctagag

SEQ ID NO: 27 - DNA, 1375 bp, Artificial Sequence
  Description of Artificial Sequence RARbeta2 PCR nucleotide product
actgccgcgggccaccatgtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgccagt

SEQ ID NO: 28 - DNA, 1399 bp, Artificial Sequence
  Description of Artificial Sequence FLAG RARbeta2 PCR nucleotide product
actgccgcgggccaccatggactacaaggacgacgatgacaagtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgccagt

SEQ ID NO: 29 - DNA, 9127 bp, Artificial Sequence
  Description of Artificial Sequence pONY-RARbeta2 vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagagtcgacgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatggtcgactctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcgggccaccatgtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgcgactctagagtcgacctcgagggggggcccggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 30 - DNA, 9151 bp, Artificial Sequence
  Description of Artificial Sequence pONY-FLAG-RARbeta2 vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagagtcgacgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatggtcgactctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcgggccaccatggactacaaggacgacgatgacaagtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgcgactctagagtcgacctcgagggggggcccggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 31 - DNA, 8528 bp, Artificial Sequence
  Description of Artificial Sequence pONY8G 5′cPPT POS delCTS EIAV vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagagtcgacgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatggtcgactctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctcgagggggggcccggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 32 - DNA, 5993 bp, Artificial Sequence
  Description of Artificial Sequence pClneoERev, EIAV Rev expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtaacggccgccagtgtgctggaattcggcttatggcagaatcgaaggaagcaagagaccaagaaatgaacctgaaagaagaatctaaagaagaaaaaagaagaaatgactggtggaaaatagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgaagccgaattctgcagatatccatcacactggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 33 - DNA, 517 bp, Artificial Sequence
  Description of Artificial Sequence codon optimised EIAV Rev expression cassette with flanking restriction endonuclease sites
gaattcgccaccatggctgagagcaaggaggccagggatcaagagatgaacctcaaggaagagagcaaagaggagaagcgccgcaacgactggtggaagatcgacccacaaggccccctggagggggaccagtggtgccgcgtgctgagacagtccctgcccgaggagaagattcctagccagacctgcatcgccagaagacacctcggccccggtcccacccagcacacaccctccagaagggataggtggattaggggccagattttgcaagccgaggtcctccaagaaaggctggaatggagaattaggggcgtgcaacaagccgctaaagagctgggagaggtgaatcgcggcatctggagggagctctacttccgcgaggaccagaggggcgatttctccgcatggggaggctaccagagggcacaagaaaggctgtggggcgagcagagcagcccccgcgtcttgaggcccggagactccaaaagacgccgcaaacacctgtgaagtcgac

SEQ ID NO: 34 - DNA, 11131 bp, Artificial Sequence
  Description of Artificial Sequence pONY4.0Z nucleotide sequence
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggcagatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - DNA, 19 bp, Artificial Sequence
  Description of Artificial Sequence Primer
tgtagctctctgagcactc

SEQ ID NO: 36 - DNA, 19 bp, Artificial Sequence
  Description of Artificial Sequence Primer
tacgccttcttctttcccc

SEQ ID NO: 37 - DNA, 21 bp, Artificial Sequence
  Description of Artificial Sequence Primer
cttttataaccagaaccgggc

SEQ ID NO: 38 - DNA, 21 bp, Artificial Sequence
  Description of Artificial Sequence Primer
caagtagaagccaggaaagtc

SEQ ID NO: 39 - DNA, 21 bp, Artificial Sequence
  Description of Artificial Sequence Primer
ctaagaagacccacacttctg

SEQ ID NO: 40 - DNA, 19 bp, Artificial Sequence
  Description of Artificial Sequence Primer
aagtgaggtgaaaactggg

SEQ ID NO: 41 - DNA, 18 bp, Artificial Sequence
  Description of Artificial Sequence Primer
ttcacagcctggcataac

SEQ ID NO: 42 - DNA, 19 bp, Artificial Sequence
  Description of Artificial Sequence Primer
gagaaggaagtgagccatc

SEQ ID NO: 43 - DNA, 21 bp, Artificial Sequence
  Description of Artificial Sequence Primer
tctctgtgcattcctgctttg

SEQ ID NO: 44 - DNA, 20 bp, Artificial Sequence
  Description of Artificial Sequence Primer
tggacacatgactcactacc

SEQ ID NO: 45 - DNA, 20 bp, Artificial Sequence
  Description of Artificial Sequence Primer
atgttctgtcagtgagtccc

SEQ ID NO: 46 - DNA, 19 bp, Artificial Sequence
  Description of Artificial Sequence Primer
gcatgtcagaggacaactg

SEQ ID NO: 47 - DNA, 18 bp, Artificial Sequence
  Description of Artificial Sequence Primer
agcctggaaaatgccatc

SEQ ID NO: 48 - DNA, 22 bp, Artificial Sequence
  Description of Artificial Sequence Primer
ttacagcttccttggacatgcc

SEQ ID NO: 49 - DNA, 20 bp, Artificial Sequence
  Description of Artificial Sequence Primer
agatgctgagccctagcttc

SEQ ID NO: 50 - DNA, 20 bp, Artificial Sequence
  Description of Artificial Sequence Primer
ttactacgcagagccactgg

SEQ ID NO: 51 - DNA, 19 bp, Artificial Sequence
  Description of Artificial Sequence Primer
ggaagatggaagagggaac

SEQ ID NO: 52 - DNA, 19 bp, Artificial Sequence
  Description of Artificial Sequence Primer
caaatttactgggggttgg

SEQ ID NO: 53 - DNA, 20 bp, Artificial Sequence
  Description of Artificial Sequence Primer
ggctggattttggattgaag

SEQ ID NO: 54 - DNA, 22 bp, Artificial Sequence
  Description of Artificial Sequence Primer
ttctgtcctctcactaccttgg

SEQ ID NO: 55 - DNA, 20 bp, Artificial Sequence
  Description of Artificial Sequence Primer
cattaccgcgagtcactaac

SEQ ID NO: 56 - DNA, 20 bp, Artificial Sequence
  Description of Artificial Sequence Primer
cgtagacaaaatggtgaagg

SEQ ID NO: 57 - DNA, 20 bp, Artificial Sequence
  Description of Artificial Sequence Primer
gactccacgacatactcagc

SEQ ID NO: 58 - DNA, 18 bp, Artificial Sequence
  Description of Artificial Sequence Primer
gcttcttcattgacccac

SEQ ID NO: 59 - DNA, 20 bp, Artificial Sequence
  Description of Artificial Sequence Primer
cttcaccgtcaggtctttac

SEQ ID NO: 60 - DNA, 20 bp, Artificial Sequence
  Description of Artificial Sequence Primer
gcaaggaccggaatgagaac

SEQ ID NO: 61 - DNA, 21 bp, Artificial Sequence
  Description of Artificial Sequence Primer
tctaggggcagctcagaaaag

SEQ ID NO: 62 - DNA, 21 bp, Artificial Sequence
  Description of Artificial Sequence Primer
agaataaaggggtagtgaagg

SEQ ID NO: 63 - DNA, 18 bp, Artificial Sequence
  Description of Artificial Sequence Primer
catcaatgtccccacttg

SEQ ID NO: 64 - DNA, 20 bp, Artificial Sequence
  Description of Artificial Sequence Primer
tgccagtagtagccacgaag

SEQ ID NO: 65 - DNA, 19 bp, Artificial Sequence
  Description of Artificial Sequence Primer
tgagcagttcattccaccc

SEQ ID NO: 66 - DNA, 5961 bp, Artificial Sequence
  Description of Artificial Sequence pESYNREV, codon-optimised EIAV Rev expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccaccatggctgagagcaaggaggccagggatcaagagatgaacctcaaggaagagagcaaagaggagaagcgccgcaacgactggtggaagatcgacccacaaggccccctggagggggaccagtggtgccgcgtgctgagacagtccctgcccgaggagaagattcctagccagacctgcatcgccagaagacacctcggccccggtcccacccagcacacaccctccagaagggataggtggattaggggccagattttgcaagccgaggtcctccaagaaaggctggaatggagaattaggggcgtgcaacaagccgctaaagagctgggagaggtgaatcgcggcatctggagggagctctacttccgcgaggaccagaggggcgatttctccgcatggggaggctaccagagggcacaagaaaggctgtggggcgagcagagcagcccccgcgtcttgaggcccggagactccaaaagacgccgcaaacacctgtgaagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

(19) United States
(12) Patent Application Publication
(10) Pub. No.: US 2004/0266715 A1
(43) Pub. Date: Dec. 30, 2004

(54) Neurite regeneration

(76) Inventors: Liang Fong Wong, Oxford (GB); Nicholas Mazarakis, Oxford (GB); Alan Kingsman, Oxford (GB); Stephen McMahon, London (GB); Malcolm Maden, London (GB)

Correspondence Address: FROMMER LAWRENCE & HAUG, 745 FIFTH AVENUE- 10TH FL., NEW YORK, NY, 10151, US

(21) Appl. No.: 10/838,906

(22) Filed: May 3, 2004

Related U.S. Application Data
(63) Continuation-in-part of application No. 10/239,804, filed on Sep. 23, 2002.
Application No. 10/239,804 is a 371 of international application No. PCT/GB01/01478, filed on Mar. 30, 2001.
(63) Continuation-in-part of application No. 09/937,716, filed on Jul. 1, 2002.
Application No. 09/937,716 is a 371 of international application No. PCT/GB00/01211, filed on Mar. 30, 2000.
(63) Continuation-in-part of application No. 10/716,725, filed on Nov. 19, 2003.
Application No. 10/716,725 is a continuation-in-part of application No. 10/429,608, filed on May 5, 2003.
Application No. 10/429,608 is a continuation-in-part of application No. PCT/GB01/04866, filed on Nov. 2, 2001.
Application No. 10/716,725 is a continuation-in-part of application No. PCT/GB03/00426, filed on Oct. 3, 2003.

(30) Foreign Application Priority Data
Oct. 4, 2000 (GB) 0024300.6
Nov. 3, 2000 (GB) 0026943.1
Jan. 30, 2001 (GB) 0102339.9
Sep. 14, 2001 (GB) 0122238.9
Oct. 4, 2002 (GB) 0223076.1
Dec. 4, 2002 (GB) 0228314.1
Aug. 4, 2003 (GB) 0318213.6
Mar. 31, 1999 (GB) 9907461.9

(51) Int. Cl. A61K 48/00

(52) U.S. Cl. 514/44

(57) ABSTRACT

The present invention relates to the use of RARβ2 and/or an agonist thereof in the preparation of a medicament to cause neurite development, neurite growth and/or neurite regeneration.

### CROSS REFERENCE TO RELATED APPLICATIONS

This application is a continuation-in-part of U.S. application Ser. No. 10/239,804, filed on Sep. 23, 2002, which is a 371 of PCT/GB01/01478, filed on Mar. 30, 2001 and claiming priority to PCT/GB00/01211, filed on Mar. 30, 2000, which claims priority to GB application Serial No. 0024300.6, filed on Oct. 4, 2000. This application is also a continuation-in-part of U.S. application Ser. No. 09/937,716, filed on Jul. 1, 2002, which is a 371 of PCT/GB00/01211, filed on Mar. 30, 2000 and claiming priority to GB application 9907461.9 filed on Mar. 31, 1999. This application is also a continuation-in-part of U.S. application Ser. No. 10/716,725, filed on Nov. 19, 2003, which is a continution-in-part of U.S. application Ser. No. 10/429,608, filed on May 5, 2003. U.S. application Ser. No. 10/429,608 is a continuation-in-part of International application no. PCT/GB01/04866, filed on Nov. 2, 2001, and claiming priority from GB application nos. 0026943.1, filed on Nov. 3, 2000, 0102339.9, filed on Jan. 30, 2001 and 0122238.9 filed on Sep. 14, 2001. U.S. application Ser. No. 10/429,608 is also a continuation-in-part of International application no. PCT/GB03/00426, filed on Oct. 3, 2003, and claiming priority from GB application nos. 0223076.1, filed on Oct. 4, 2002, 0228314.1, filed on Dec. 4, 2002 and 0318213.6, filed on Aug. 4, 2003.

This application makes reference to U.S. application Ser. No. 09/701,014, filed on Nov. 22, 2000, Ser. No. 09/224,014, filed on Dec. 28, 1998, Ser. No. 09/915,169, filed on Jul. 25, 2001, Ser. No. 10/661,761, filed on Sep. 11, 2003, Ser. No. 09/238,356, filed on Jan. 27, 1999, Ser. No. 10/408,456, filed on Apr. 7, 2003 and 10/258,089, filed on Oct. 18, 2002.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a factor relating to neurite growth. Furthermore, the invention relates to vectors capable of directing the expression of a factor, such as Retinoic Acid Receptor (RAR) β2, relating to neurite growth.

### BACKGROUND OF THE INVENTION

The human peripheral and central nervous system consists of terminally differentiated cells which are not capable of directing neurite outgrowth or neurite regeneration.

It is desirable to cause neurite development, such as neurite outgrowth and/or neurite regeneration, for example in cases of nervous injuries such as avulsion injuries or spinal cord injuries or in diseases such as diabetes or neuropathies.

Nerve growth factor (NGF) is known to stimulate certain events such as neurite outgrowth. However, NGF is a relatively large molecule with a correspondingly high molecular weight. Moreover, NGF is susceptible to protease mediated degradation. Due to these and other considerations, NGF is difficult to administer. NGF is also relatively expensive to prepare. These are problems associated with the prior art.

### SUMMARY OF THE INVENTION

We have surprisingly found that it is possible to cause neurite development, such as neurite outgrowth and/or neurite regeneration, by using retinoic acid receptor β2 (RARβ2) and/or an agonist thereof. Moreover, it is surprisingly shown that RARβ2 can be delivered to non-dividing mammalian cells using vectors according to the invention.

The present invention is based on the surprising finding that it is possible to cause neurite development, such as neurite outgrowth and/or neurite regeneration, by using RARβ2 and/or an agonist thereof, and that RARβ2 may be introduced into neuronal cells using retroviral vectors based on lentiviral vectors.

Aspects of the present invention utilise this finding. For example it is possible to have a method that causes modulation of neurite development, such as neurite outgrowth and/or neurite regeneration, by using RARβ2 and/or a vector comprising same and/or an agonist thereof as explained herein.

In one aspect, the present invention relates to a viral vector comprising a nucleic acid sequence encoding a receptor such as RARβ2.

The viral vector may be based on or derived from a DNA virus, or an RNA virus (a retrovirus). Examples of such viral vectors include but are not limited to herpes viruses, adenoviruses, adeno-associated viruses, retroviruses, lentiviruses and other viruses. This is discussed in more detail below.

The receptor may be any eukaryotic receptor, such as a vertebrate receptor. Examples of such receptors include but are not limited to mammalian receptors, primate receptors and human receptors. This is explained more fully in the following section(s).

In another aspect, the present invention relates to a retroviral vector derived from a lentivirus genome comprising a nucleic acid sequence capable of directing the expression of a receptor.

In another aspect, the present invention relates to a viral vector comprising a nucleic acid sequence encoding the retinoic acid receptor β2 (RARβ2).

In another aspect, the present invention relates to a retroviral vector derived from a lentivirus genome comprising a nucleic acid sequence capable of directing the expression of the retinoic acid receptor β2 (RARβ2).

In another aspect, the present invention relates to a gene therapy vector comprising a nucleic acid sequence encoding a retinoic acid receptor β2. In a preferred aspect, delivery of the nucleic acid encoding the retinoic acid receptor β2 enables neurite growth and/or neurite regeneration.

In another aspect, the present invention relates to the use of a vector as described herein in the preparation of a medicament to cause neurite development and/or neurite regeneration.

In another aspect, the present invention relates to the use of a vector as described herein in the preparation of a medicament for the treatment of a neurological disorder such as nerve injuries.

In another aspect, the present invention relates to a method of treating a neurological disorder such as nerve injuries comprising administering a vector as described herein to a subject.

In another aspect, the present invention relates to a host or target cell when transduced by a vector as described herein.

In another aspect, the present invention relates to a pharmaceutical composition comprising a vector as described herein in admixture with a pharmaceutically acceptable carrier, diluent or excipient; wherein the pharmaceutical composition is for use to cause neurite development and/or neurite regeneration.

In another aspect, the present invention relates to the use of RARβ2 and/or an agonist thereof in the preparation of a medicament to cause neurite development and/or neurite regeneration.

The term RARβ2′ as used herein may refer to the polypeptide translation product of the RARβ2 gene open reading frame (ORF), that is to say the actual receptor itself, or may refer to the nucleic acid ORF encoding said polypeptide, or may even occasionally refer to the RARβ2 gene itself. It will be apparent to the reader which of these entities, or combination of said entities, is referred to by the term ‘RARβ2’ from the particular context in which such term is used.

In the present invention the RARβ2 and/or an agonist can be termed a pharmaceutically active agent.

Neurites are well known structures which develop from various neuronal cell types. They appear as microscopic branch or comb-like structures or morphological projections from the surface of the cell from which they emanate. Since it is usually difficult to distinguish a dendrite from an axon in culture, the term neurite is usually used for both. Examples of neurite outgrowth and neurite regeneration are shown in the accompanying figures, and in publications such as those referenced in (Maden 1998-review article), and are well known in the art. In addition, neurite regeneration will include growth or regeneration of neurons and/or nerve fibers. Nerve fibers refer to threadlike extensions of a nerve cell, and are essentially the axon of a nerve cell, ensheathed by oligodendroglia cells in the brain and spinal cord, and by Schwann cells in the peripheral nerves.

The RARβ2 coding sequence (i.e. the RARβ2 gene) is used as described hereinbelow. The RARβ2 gene may be prepared by use of recombinant DNA techniques and/or by synthetic techniques. For example, it may be prepared using the PCR amplified gene fragment prepared as in the Examples section of this document using the primers etc. detailed therein, or it may be prepared according to any other suitable method known in the art.

In another aspect, the present invention relates to the use of RARβ2 and/or an agonist thereof in the preparation of a medicament to cause neurite development and/or neurite regeneration, wherein said agonist is retinoic acid (RA) and/or CD2019.

Retinoic acid is commercially available. CD2019 is a polycyclic heterocarbyl molecule which is a RARβ2 agonist having the structure as discussed herein and as shown in (Elmazar et al., (1996) Teratology vol. 53 pp158-167).

In another aspect, the present invention relates to the use of RARβ2 and/or an agonist thereof in the preparation of a medicament for the treatment of a neurological disorder such as a nerve injury.

In another aspect, the present invention relates to the use of RARβ2 and/or an agonist thereof in the preparation of a medicament for the treatment of a neurological disorder, wherein said neurological disorder comprises neurological injury such as an avulsion injury or a spinal cord injury. An avulsion injury is the tearing away of nerves as a result of an injury.

In another aspect, the present invention relates to a method of treating a neurological disorder such as a nerve injury comprising administering a pharmacologically active amount of an RARβ2 receptor, and/or an agonist thereof.

In another aspect, the present invention relates to a method of treating a neurological disorder such as a nerve injury comprising administering a pharmacologically active amount of an RARβ2 receptor, and/or an agonist thereof, wherein said agonist is RA and/or CD2019.

In another aspect, the present invention relates to a method of treating a neurological disorder such as a nerve injury comprising administering a pharmacologically active amount of an RARβ2 receptor, and/or an agonist thereof, wherein said RARβ2 receptor is administered by an entity comprising a RARβ2 expression system.

In another aspect, the present invention relates to a method of causing neurite development or neurite regeneration in a subject, said method comprising providing a nucleic acid construct capable of directing the expression of at least part of a RARβ2 receptor, introducing said construct into one or more cells of said subject, and optionally administering a RARβ2 agonist, such as RA and/or CD2019, to said subject.

In a further aspect, the invention relates to an assay method for determining whether an agent is capable of modulating RARβ2 signalling, said method comprising providing target neural cells, contacting said cells with said agent, and assessing the activity of the RARβ2 receptor, such as through the monitoring of neurite outgrowth and/or neurite regeneration.

Neural cells for use in the assay method of the invention may be any suitable neural cell line, whether stably maintained in culture, or primary cells derived from an animal directly. Preferably said cells will be embryonic mouse dorsal root ganglion (DRG) cells prepared as described below.

In a further aspect, the invention relates to a process comprising the steps of (i) performing the assay for modulation of RARβ2 signalling described above, (ii) identifying one or more agents that are capable of modulating said RARβ2 signalling, and (iii) preparing a quantity of those one or more identified agents.

In a further aspect, the invention relates to a process comprising the steps of (i) performing the assay for modulation of RARβ2 signalling described above, (ii) identifying one or more agents that are capable of modulating said RARβ2 signalling, (iii) preparing a quantity of those one or more identified agents, and (iv) preparing a pharmaceutical composition comprising those one or more identified agents.

In a further aspect, the invention relates to a method of affecting the in vivo activity of RARβ2 with an agent, wherein the agent is capable of modulating RARβ2 signalling, for example capable of modulating RARβ2 signalling in an in vitro assay method as described above.

In a further aspect, the invention relates to the use of an agent in the preparation of a pharmaceutical composition for the treatment of a neurological disorder or nerve injury, wherein the agent is capable of modulating RARβ2 signalling, for example capable of modulating RARβ2 signalling in an in vitro assay method as described above.

In a further aspect, the invention relates to a method of treating a subject with an agent, wherein the agent is capable of modulating RARβ2 signalling, for example capable of modulating RARβ2 signalling in an in vitro assay method as described above.

In a further aspect, the invention relates to a pharmaceutical composition comprising RARβ2 and/or an agonist thereof in admixture with a pharmaceutically acceptable carrier, diluent or excipient; wherein the pharmaceutical composition is for use to cause neurite development and or neurite regeneration.

In a further aspect of the invention, there is provided a viral vector genome comprising nucleic acid sequence(s) capable of directing the expression of a receptor. Preferably said vector genome comprises nucleic acid sequence(s) capable of directing the expression of at least part of the RARβ2 receptor.

In a further aspect of the invention, there is provided a retroviral vector genome comprising nucleic acid sequence(s) capable of directing the expresion of at least part of RARβ2, said genome containing a deleted gag gene from a lentivirus wherein the deletion in gag removes one or more nucleotides downstream of nucleotide 350 of the gag coding sequence. Preferably the deletion extends from nucleotide 350 to at least the C-terminus of the gagpol coding region. More preferably the deletion additionally removes nucleotide 300 of the gag coding region and most preferably the deletion retains only the first 150 nucleotides of the gag coding region. However even larger deletions of gag can also be used, for example the gag coding region may contain only the first 109 nucleotides of the gag coding region. It may also be possible for the gag coding region to contain only the first 2 nucleotides of the gag coding region. Preferably, said vector genome is capable of directing the expression of substantially all of the RARβ2 polypeptide.

Preferably, the vector of the present invention is based on or derived from a lentivirus. More preferably, the vector of the present invention is based on or derived from a non-primate lentivirus. In a highly preferred embodiment, the vector of the present invention is based on or derived from a non-primate lentivirus such as equine infectious anaemia virus (EIAV).

This is discussed in more detail below.

Additional features of the lentiviral genome are included in the vector genome which are necessary for transduction of the target cell such as reverse transcription and integration. These are, at least, a portion of an LTR containing sequence from the R-region and U5 region, sequences adjacent to the 3′ LTR which contain a polypurine tract (PPT) and a 3′LTR from the lentivirus or a hybrid LTR containing sequences from the lentivirus and other elements. Optionally, the retroviral genome may contain accessory genes derived from a retrovirus, such as, but not limited to, a rev gene, a tat gene, a vif gene, a nef gene, a vpr gene or an S2 gene. Additional components may be added such as introns, splice-donor sites, a rev responsive element (RRE), sequences called the cPPT containing the polymerase region (Stetor S R, Rausch J W, Guo M J, Burnham J P, Boone L R, Waring M J, Le Grice S F ‘Characterization of (+) strand initiation and termination sequences located at the center of the equine infectious anemia virus genome.’ Biochemistry. 1999 Mar. 23;38(12):3656-67), cloning sites and selectable marker genes.

Moreover, it has been demonstrated (e.g. see WO 99/32646) that a lentivirus minimal vector system can be constructed which requires neither S2, Tat, env nor dUTPase for either vector production or for transduction of dividing and non-dividing cells. A lentivirus minimal vector system can also be constructed which requires neither S2, Tat, env, rev nor dUTPase for either vector production or for transduction of dividing and non-dividing cells. A minimal lentiviral vector system is a lentiviral vector system comprising no accessory genes with the optional exception of the rev gene.

The lentiviral vector is advantageously as in U.S. Pat. Nos. 6,312,683, 6,312,682 or in other patent documents (e.g., applications) incorporated herein by reference wherein the assignee or applicant (e.g., on PCT and UK applications) is Oxford Biomedica, such as UK application serial No. 0024550.6 and PCT/GB01/04433, which can also be sources for additional vectors or additional coding sequences to be employed in the practice of the invention.

Thus according to another aspect the lentivirus genome from which the vector is derived lacks one or more accessory genes.

The use of a minimal lentiviral vector system, i.e., the deletion of accessory genes is highly advantageous. Firstly, it permits vectors to be produced without the genes normally associated with disease in lentiviral (e.g. HIV) infections. In particular, tat and nef are associated with disease. Secondly, the deletion of accessory genes permits the vector to package more heterologous DNA. Thirdly, genes whose function is unknown, such as dUTPase and S2, may be omitted, thus reducing the risk of causing undesired effects.

In addition, we have shown that the leader sequence of the lentivirus genome is preferable for high protein expression.

Therefore in a further aspect the lentivirus genome from which the vector is derived lacks the tat gene but includes the leader sequence between the end of the 5′ LTR and the ATG of gag.

These data further define a minimal essential set of functional components for an optimal lentiviral vector. A vector is provided with maximal genetic capacity and high titre, but without accessory genes that are either of unknown function (S2, UTPase), and therefore may present risk, or are analogues of HIV proteins that may be associated with AIDS (tat, rev).

It will be appreciated that the present invention provides a retroviral vector derived from a lentivirus genome comprising nucleic acid sequence capable of directing the expression of at least part of RARβ2 and (1) comprising a deleted gag gene wherein the deletion in gag removes one or more nucleotides downstream of nucleotide 350 of the gag coding sequence; (2) wherein one or more accessory genes are absent from the lentivirus genome; (3) wherein the lentivirus genome lacks the tat gene but includes the leader sequence between the end of the 5′ LTR and the ATG of gag; and combinations of (1), (2) and (3). In a preferred embodiment the retroviral vector comprises all of features (1) and (2) and (3).

A “non-primate” vector, as used herein, refers to a vector derived from a virus which does not primarily infect primates, especially humans. Thus, non-primate virus vectors include vectors which infect non-primate mammals, such as dogs, sheep and horses, reptiles, birds and insects.

A lentiviral or lentivirus vector, as used herein, is a vector which comprises at least one component part derived from a lentivirus. Preferably, that component part is involved in the biological mechanisms by which the vector infects cells, expresses genes or is replicated.

The lentivirus may be any member of the family of lentiviridae. Preferably the lentivirus is one which does not naturally infect a primate (‘non-primate lentivirus’). Such viruses may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). Preferably the lentivirus is an EIAV. Equine infectious anaemia virus infects all equidae resulting in plasma viremia and thrombocytopenia (Clabough, et al. 1991. J. Virol. 65:6242-51). Virus replication is thought to be controlled by the process of maturation of monocytes into macrophages.

EIAV has the simplest genomic structure of the lentiviruses. In addition to the gag, pol and env genes EIAV encodes three other genes: tat, rev, and S2. Tat acts as a transcriptional activator of the viral LTR (Derse and Newboldl993 Virology. 194:530-6; Maury, et al 1994 Virology. 200:632-42.) and Rev regulates and coordinates the expression of viral genes through rev-response elements (RRE) (Martarano et al 1994 J. Virol. 68:3102-11.). The mechanisms of action of these two proteins are thought to be broadly similar to the analogous mechanisms in the primate viruses (Martano et al ibid). The function of S2 is unknown. In addition, an EIAV protein, Ttm, has been identified that is encoded by the first exon of tat spliced to the env coding sequence at the start of the transmembrane protein.

In addition to protease, reverse transcriptase and integrase lentiviruses contain a fourth pol gene product which codes for a dUTPase. This may play a role in the ability of these lentiviruses to infect certain non-dividing cell types.

The viral RNA in aspect(s) of the invention is transcribed from a promoter, which may be of viral or non-viral origin, but which is capable of directing expression in a eukaryotic cell such as a mammalian cell. Optionally an enhancer is added, either upstream of the promoter or downstream. The RNA transcript is terminated at a polyadenylation site which may be the one provided in the lentiviral 3′ LTR or a different polyadenylation signal.

Thus the present invention provides a DNA transcription unit comprising a promoter and optionally an enhancer capable of directing expression of a retroviral vector genome. Such promoters and enhancers may be constitutive, inducible, or tissue-specific.

Transcription units as described herein comprise regions of nucleic acid containing sequences capable of being transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns).

In another aspect, the present invention relates to a retroviral vector derived from a lentivirus genome comprising a nucleic acid sequence capable of directing the expression of at least part of RARβ2 and comprising a deleted gag gene wherein the deletion in gag removes one or more nucleotides downstream of nucleotide 350 of the gag coding sequence.

In another aspect, the present invention relates to a retroviral vector as described herein, wherein the deletion extends from nucleotide 350 to at least the C-terminus of the gag-pol coding region.

In another aspect, the present invention relates to a retroviral vector as described herein, wherein the deletion additionally removes nucleotide 300 of the gag coding region.

In another aspect, the present invention relates to a retroviral vector as described herein, wherein the deletion retains the first 150 nucleotides of the gag coding region.

In another aspect, the present invention relates to a retroviral vector as described herein, wherein the deletion retains the first 109 nucleotides of the gag coding region.

In another aspect, the present invention relates to a retroviral vector as described herein, wherein the deletion retains only the first 2 nucleotides of the gag coding region.

In another aspect, the present invention relates to a retroviral vector as described herein, wherein the deletion is of the entire gag coding region.

In another aspect, the present invention relates to a retroviral vector derived from a lentivirus genome wherein one or more accessory genes are absent from the lentivirus genome.

In another aspect, the present invention relates to a retroviral vector as described herein, wherein the accessory genes are selected from dUTPase, S2, rev and tat.

In another aspect, the present invention relates to a retroviral vector derived from a lentivirus genome such as EIAV wherein the lentivirus genome lacks the tat gene but includes the leader sequences between the end of the 5′ LTR and the ATG of gag.

In another aspect, the present invention relates to a retroviral vector as described herein, which comprises at least one component from an equine lentivirus.

In another aspect, the present invention relates to a retroviral vector as described herein, wherein the equine lentivirus is EIAV.

In another aspect, the present invention relates to a retroviral vector as described herein, wherein the retroviral vector is substantially derived from EIAV.

In another aspect, the present invention relates to a method comprising transfecting or transducing a cell with a retroviral vector as described herein.

In another aspect, the present invention relates to a delivery system in the form of a retroviral vector as described herein.

In another aspect, the present invention relates to a cell transfected or transduced with a retroviral vector as described herein.

In another aspect, the present invention relates to use of a retroviral vector as described herein.

In another aspect, the present invention relates to use of a gene therapy vector as described herein.

In another aspect, the invention relates to the use of lentiviral gene therapy vectors for the delivery of retinoic acid receptor (RAR)β2 to the peripheral and central nervous systems.

In another aspect, the present invention relates to a gene therapy vector comprising a nucleic acid sequence encoding a retinoic acid receptor (RAR)β2. In a preferred aspect, delivery of the nucleic acid encoding the retinoic acid receptor (RAR)β2 enables neurite growth and/or neurite regeneration.

In another aspect, the invention relates to EIAV gene therapy vectors configured to express retinoic acid receptor β2 (RARβ2).

In another aspect, the invention relates to methods for producing expression of RARβ2 in adult mammalian (such as human) spinal cord. Expression of RARβ2 in adult spinal cord is shown to stimulate neurite outgrowth and regeneration. Thus, in a preferred aspect, the invention relates to methods for stimulation of neurite outgrowth and/or regeneration in mammalian neuronal cells. In addition, the invention relates to methods for producing RARβ2 in nerve injury such as avulsion and spinal cord injury wherein expression of RARβ2 results in a therapeutic effect such as regeneration of neurons or nerve fibers, promotion of neurite outgrowth, reduction of inflammation, reduction of apoptotic cell death, promotion of sensory recovery, and promotion of locomotor function recovery.

As used herein, the term ‘adult’ is used to mean non-foetal and/or non-embryonic. The term thus includes adults per se, as well as including young such as children and/or pups or other such infants. Thus, the term ‘adult’ as used herein may be understood to include any ‘post-natal’ ie. post-birth organism.

In another aspect, the invention relates to a differential expression screening method for identifying genes involved in a cellular process which method comprises comparing gene expression in: a first cell of interest; and a second cell of interest which cell comprises altered levels, relative to physiological levels, of a biological molecule due to the introduction into the second cell of a heterologous nucleic acid encoding at least part of RARβ2; and identifying gene products whose expression differs. Preferably, said heterologous nucleic acid encodes substantially all of RARβ2. Optionally, retinoic acid or an analogue thereof may also be present in the cellular environment of one or preferably both cells of interest. This method or a variant thereof may be advantageously applied to comparison of non-dividing neuronal cells with a different sample of the same cells which have been induced to exhibit neurite outgrowth, such as via transduction with a vector delivering RARβ2, or via other techniques discussed herein.

For ease of reference, these and further aspects of the present invention are now discussed under appropriate section headings. However, the teachings under each section are not necessarily limited to each particular section.

In a preferred aspect, the administration of a nucleic acid construct capable of directing the expression of RARβ2 will be accompanied by the administration of a RARβ2 agonist such as RA, or preferably CD2019 (or a mimetic thereof).

Preferably said agonist will be to some degree selective for the RARβ2 receptor. Preferably said agonist will not significantly affect the RARα receptor. Preferably said agonist will not significantly affect the RARγ receptor. More preferably said agonist will not significantly affect the RARα receptor or the RARγ receptor. Even more preferably, said agonist will exhibit a high degree of selectivity for the RARβ2 receptor.

In a preferred aspect, the administration of a nucleic acid construct capable of directing the expression of RARβ2 will be accomplished using a vector, preferably a viral vector, more preferably a retroviral vector such as a lentiviral vector. In a highly preferred embodiment, the administration of a nucleic acid construct capable of directing the expression of RARβ2 will be accomplished using a retroviral vector capable of infecting non-dividing mammalian cells such as neural cells. This retroviral vector will preferably be derived from a lentiviral vector (preferably a non-primate lentiviral vector as discussed above), more preferably said vector will be derived from an equine infectious anaemia virus (EIAV). In a highly preferred aspect, the lentiviral vector is a minimal lentiviral vector. In a highly preferred aspect, said EIAV-derived vector will be a pseudotyped particle, such as VSV-G pseudotyped, or Rabies G pseudotyped.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIGS. 1a-1h show neurite outgrowth in adult mouse DRG cultured for five (1a-1d, 1g, 1h) or eight days (1e, 1f) in the presence of delipidated serum plus: (1a) no addition; (1b) NGF, 100 ng per ml; (1c) NGF and 100 nM tRA; (1d) NGF and 10 M disulphiram; (1e) disulphiram and tRA added on day 0; (1f) disulphiram; (1g) NGF and blocking antibody (1h) NGF-blocking antibody and tRA.

FIGS. 2a-2c show neurites produced by adult DRG cultured in cellogen. FIG. 2a shows the effects of NGF, RA and disulphiram at five days (1, no additive; 2, NGF, 100 ng per ml; 3, RA, 100 nM; 4, NGF, 100 ng per ml and RA, 100 nM; 5, 100 ng/ml NGF and 10 M disulphiram; 6, NGF, 100 ng per ml and DMSO). Error bars, s.e.; n=6, all groups.

Differences between NGF-treated (2) and other groups: *p<0.01; **p<0.0001; Student's t-test.

FIG. 2b shows RA rescue of DRG treated with 10 M disulphiram (left to right: no RA; 100 nM RA, day 0; 100 nM RA, day 4) Error bars, s.e.; n=6, all groups. Differences from RA-absent cultures: *p<0.01, **p<0.0001; Student's t-test. FIG. 2c shows the effect of NGF-blocking antibody on 5-day DRG cultures. Left, NGF, 100 ng per ml; center, NGF plus blocking antibody; right, blocking antibody plus 100 nM RA; n=4. Differences from NGF plus blocking antibody: *p<0.01, **p<0.0001, Student's t-test. FIG. 2d shows an increase in percentage β-gal-positive F9 cells in response to DRG cultured with or without NGF. Left, no additive; center, NGF, 100 ng per ml; right, NGF with blocking antibody. Differences in percentage β-gal-positive cells from that produced by NGF-treated DRG; *p<0.025, Student's t-test; for each group, n=9. FIG. 2e shows RT-PCR analysis of RALDH-2 enzyme and RARβ8 expression in adult DRG cultured with or without NGF (100 ng per ml) for five days. GAPDH was used to indicate presence of cDNA in both samples. Use of F9 reporter cells in studying RA distribution in chick embryo has been described¹⁶.

FIGS. 3A-H show a comparison of the effect of retinoic acid on neurite outgrowth on cultured E13.5 (3A, 3C, 3E, 3G) and 10 month old adult spinal cord (3B, 3D, 3F, 3H). Pieces of spinal cord were cultured in cellogen in the presence of 10% delipidated serum and RA for a period of five days. The medium was changed every two days. 3A, 3B, no RA; 3C, 3D, 1×10⁻⁸ M RA; 3E, 3F, 1×10⁻⁷ M RA; 3G, 3H, 1×10⁻⁶ M RA.

FIGS. 4A and 4B show expression of RARβ2 in E13.5 (4A) and 10 month old adult spinal cord (4B). Pieces of spinal cord were cultured in the presence of various concentrations of RA for a period of five days after which time RT-PCR analysis of RARβ2 was performed. 4A: E13.5 (lanes 2-5) and 4B: 10 month old adult spinal cord (lanes 2-5). Lanes: 1. bluescript/HPA II size markers, 2. no RA, 3. 1×10⁻⁸ M RA, 4. 1×10⁻⁷ M RA, 5. 1×10⁻⁶ M RA. The presence of GAPDH was used to indicate equal amounts of cDNA in the samples.

FIGS. 5A and 5B show transfection of adult spinal cord with pHSVlacZ. Cultured 10 month old adult spinal cord was transfected with 5×10⁻⁴ ipu/ul pHSVlacZ overnight and analysed for β galactosidase staining 3 days later. FIG. 5A shows non-transfected adult spinal cord. FIG. 5B shows adult spinal transfected with pHSVlacZ.

FIG. 6 shows transfection of adult spinal cord with either pHSVRARβ2 or pHSVRARβ4.

Adult spinal cord was cultured in cellogen and transfected either with 5×10-4 ipu/ul of pHSVRARβ2 or 4×10-4 ipu/ul pHSVRARβ4 overnight. RT-PCR analysis four days after transfection, of RARβ2 (lanes 2-4) and RARβ4 (lanes 6-8) expression in adult spinal cord transfected with Lanes 2, 6 no virus, 3, 7 pHSVRARβ2, 4, 8, pHSVRARβ4. The presence of GAPDH was used to indicate equal amounts of cDNA in the samples. Lanes 1,2 bluescript/HPA II size markers.

FIGS. 7A-7C show the effect of either pHSVlacZ (7A), pHSVRARβ2 (7B) or pHSVRARβ4 (7C) transfection in cultured adult spinal cord on neurite outgrowth. Ten month old spinal cord was cultured in cellogen and transfected with either 5×10-4 ipu/ul, pHSVlacZ, 5×10-4 ipu/ul, pHSVRARβ2 or 4×10-4 ipu/ul pHSVRARβ4 overnight, and analysed for neurite staining with NF200 4 days after transfection.

FIG. 8 shows a barchart of the average number of neurites per spinal cord explant.

FIGS. 9A-9R show expression of the RARs and RXRs by E13.5 mouse embryo DRG neurons cultured either in the presence of NGF, NT-3 or BDNF. In situ hybridisation of: 9A-9F, NGF neurons; 9G-9L, NT-3 neurons; 9M-9R, BDNF neurons. Expression of: 9A, 9G, 9M, RARα; 9B, 9H, 9N, RARβ; 9C, 9I, 9O, RARγ, 9D, 9J, 9P. RXRα; 9E, 9K, 9Q, RXRβ; 9F, 9L, 9R, RXRγ.

FIGS. 10A-10F show the effect of RA on neurite outgrowth from DRG neurons. DRG neurons were cultured either in the presence of NGF, NT-3 or BDNF for a period of two days at which point 1×10⁻⁷ M all-trans-RA was added. They were then examined for neurite outgrowth after a total of five days with NF200 antibody. 10A, NGF; 10B, NGF+1×10⁻⁷ M RA; 10C, NT-3; 10D, NT-3+1×10⁻⁷; 10E, BDNF; 10F, BDNF+1×10⁻⁷ M RA.

FIGS. 11A-11C show expression of RARα isoforms in DRG neurons cultured either in the absence or presence of RA. DRG neurons were cultured in the presence of either NGF, NT-3 or BDNF for a period of two days, 1×10-7 M RA was then added and the presence of the RARA isoforms were then assayed by RT-PCR. Controls had no RA added. 11A, control NGF neurons lanes 1-7; NGF neurons+1×10⁻⁷ M RA lanes 8-14. 11B, control NT-3 neurons lanes 1-7; NT-3 neurons+1×10-7 M RA lanes 8-14. 11C, control BDNF neurons lanes 1-7; BDNF neurons+1×10-7 M RA lanes 8-14. Lanes: 1 & 8, RARal; 2 & 9, RARβ2; 3 & 10, RARα3; 4 & 11, RARα4; 5 & 12, RARα5; 6 & 13, RARα6; 7 & 14, RARα7.

FIGS. 12.A-12C show expression of RARβ isoforms in DRG neurons cultured either in the absence or presence of RA. DRG neurons were cultured in the presence of either NGF, NT-3 or BDNF for a period of two days, 1×10-7 M RA was then added and the presence of the RARβ isoforms were then assayed by RT-PCR. Controls had no RA added. 12A, control NGF neurons lanes 1-4; NGF neurons+1×10⁻⁷ M RA lanes 5-8. 12B, control NT-3 neurons lanes 1-4; NT-3 neurons+1×10⁻⁷ M RA lanes 5-8. 12C, control BDNF neurons lanes 1-4; BDNF neurons+1×10-7 M RA lanes 5-8. Lanes: 1 & 5, RARβ1; 2 & 6, RARβ2; 3 & 7, RARβ3; 4 & 8, RARβ4.

FIGS. 13A and 13B show expression of RARγ isoforms in DRG neurons cultured either in the absence or presence of RA. DRG neurons were cultured in the presence of either NGF, NT-3 or BDNF for a period of two days, 1×10⁻⁷ M RA was then added and the presence of the RARγ isoforms were then assayed by RT-PCR. Controls had no RA added. 13A, control NGF neurons lanes 1-7; NGF neurons+1×10-7 M RA lanes 8-14. 13B, control NT-3 neurons lanes 1-7; NT-3 neurons+1×10-7 M RA lanes 8-14. Lanes: 1 & 8, RARγ1; 2 & 9, RARγ2; 3 & 10, RARγ3; 4 & 11, RARγ4; 5 & 12, RARγ5; 6 & 13, RARγ6; 7 & 14, RARγ7.

FIGS. 14A-14L show the effect of RAR and RXR agonists on neurite outgrowth from DRG neurons. DRG neurons were cultured either in the presence of NGF, NT-3 or BDNF for a period of two days at which point either 1×10⁻⁷M of either CD366 (RARaagonist, CD2019 (RARβ agonist), CD437 (RARγ agonist) or CD2809 (pan-RXR agonist) were added to the cultures. Cultures were then stained for neurite outgrowth at five days with the NF200 antibody. 14A-14D, NGF type neurons; 14E-14H, NT-3 type neurons; 14I-14L, BDNF type neurons. Agonists: RARA 14A, 14E, 14I; RARβ3 14B, 14F, 14J; RARγ 14C, 14G, 14K; RXR 14D, 14H, 14L.

FIGS. 15A-15C show the effect of retinoid agonists on the length of neurites from 15A. NGF type neurons; 15B. NT-3 type neurons, 15C. BDNF type neurons. Columns 1. no agonist, 2. RA, 3. RARα 4. RARβ 5. RARγ 6. RXR. Error bars s.e.m., n=50. *p<0.01.

FIGS. 16A-16C show the effect of a RARγ or RARβ agonist on the expression of RARγ1, RARβ2, and GAPDH expression in DRG neurons cultured in the presence of NGF or NT-3.

DRG neurons were cultured in the presence of serum free medium. After two days 1×10⁻⁷M RARγ or RARβ agonist were then added to the cultures for a period of 24 hrs. RT-PCR analysis of 16A, RARγ1; 16B, RARβ2 expression in NGF (lanes, 1-3) and NT-3 (lanes, 4-6) type neurons. Lanes: 1,4, no agonist; 2,5, RARγ agonist; 3, 6, RARβ agonist

FIG. 17 shows chemical formula I and chemical formula II.

FIGS. 18A-18D show four photomicrographs. These photomicrographs demonstrate expression of β-galactosidase in spinal cord (18A and 18C) or DRG (18B and 18D) explants transduced with pONY8z vector particles pseudotyped with VSV-G (18A and 18B) or Rabies G (panels 18C and 18D) protein. Briefly, spinal cord (18A and 18C) and dorsal root ganglia (18B and 18D) were obtained from eight month old adult rats, placed in a cellogen matrix and treated as described above, being injected with 3 ul of virus comprising the lacZ gene and cultured in DMEM/F12 medium with 5% Foetal Calf Serum (FCS). After 5 days, they were stained for lacZ expression.

FIG. 19 shows the plasmid construct of pESYNGP.

FIG. 20 shows the plasmid construct of pONY8.0Z.

FIG. 21 shows the plasmid construct of pONY3.1.

FIG. 22 shows the plasmid construct of pONY3.1.

FIG. 23 shows the plasmid construct of E syn GP Koza.

FIG. 24 shows the plasmid construct of pE SD syn GP.

FIG. 25 shows the plasmid construct of pONY9 3′RARB2.

FIG. 26 shows the plasmid construct of pONY9 5′RARB2.

FIG. 27 shows the plasmid construct of pONY9Z 5′POS MIN.

FIG. 28 shows the plasmid construct of pONY9Z 3′POS MIN.

FIG. 29 shows the plasmid construct of pSA91RbG.

FIGS. 30A-30D show the pony 8.0Z vector genome plasmid sequence.

FIG. 31 shows the plasmid construct of pONY8.0Z.

FIGS. 32A-32D show the pONY3.1 EIAV gag/pol expression plasmid sequence.

FIG. 33 shows the plasmid construct of pONY3.1.

FIGS. 34A and 34B show the pRV67 VSV-G expression plasmid sequence.

FIG. 35 shows the plasmid construct of pRV67.

FIG. 36 shows the RARβ2 PCR product made with EX7 RARβ2 FWD and EX7 RARβ2 REV. The recognition sites for SacII and NotI and the ATG of RARβ2 are underlined.

FIG. 37 shows the FLAG RARβ2 PCR product made with EX7 RARβ2 FLAG FWD and EX7 RARβ2 REV. The recognition sites for SacII and NotI and the FLAG sequence are underlined.

FIGS. 38A-38C show the pONY RARβ2 vector genome plasmid sequence.

FIGS. 39A-39C show the pONY-FLAG-RARβ2 vector genome plasmid sequence.

FIGS. 40A-40C show the pONY8G 5′cPPT POS delCTS EIAV vector genome plasmid sequence.

FIG. 41 shows the pONY8G 5′cPPT POS delCTS plasmid construct.

FIGS. 42A-42C show the pESYNGP codon-optimised EIAV gag/pol expression plasmid sequence.

FIG. 43 shows the pESYNGP plasmid construct.

FIGS. 44A-44C show the pESDSYNGP codon-optimised EIAV gag/pol expression plasmid sequence.

FIG. 45 shows the pESDSYNGP plasmid construct.

FIGS. 46A and 46B show the pCIneoERev EIAV Rev expression plasmid sequence.

FIG. 47 shows the pCIneoERev plasmid construct.

FIGS. 48A and 48B show the pESYNREV codon-optimised EIAV Rev expression plasmid sequence.

FIG. 49 shows the pESYNREV plasmid construct.

FIGS. 50A and 50B show transduction of EIAVLacZ pseudotyped with VSV-G envelope into the rat spinal cord. Micrographs show X-gal histochemistry. FIG. 50B shows high magnification of the ventral hom.

FIGS. 51A-51C show transduction of EIAVLacZ pseudotyped with VSV-G envelope into the spinal cord. The photomicrographs show NeuN/β-gal double immunostaining. β-gal staining shows as green in 51A (marked ‘β-gal’), NeuN staining shows as red in 51B, and both red and green channels are shown in the β-gal/NeuN double stain in 51C.

FIGS. 52A-52C show staircase testing in the cervical rhizotomy model. Graphs illustrate the number of food pellets retrieved with either the left or right forelimb before or after injury in the LacZ-(52A), RARβ2-(52B) or GDNF-(52C) transduced groups.

FIGS. 53A-53D show tape sensing and removal test in the cervical rhizotomy model.

Graphs illustrate the time taken for the animals to sense (test for sensory function) and remove (test for motor function) the adhesive tape on the left and right paw.

FIGS. 54A-54C show ladder crossing test in the cervical rhizotomy model. The number of footslips made with both limbs and the time taken to cross the ladder were recorded.

FIG. 55 shows beam crossing in the cervical rhizotomy model.

FIGS. 56A-56D show footprint analysis in the cervical rhizotomy model. The stride lengths and limb widths of the left and right forelimb are illustrated.

FIGS. 57A and 57B show tape sensing and removal in the CST lesion study.

FIGS. 58A and 58B show beam crossing in the CST model.

FIGS. 59A and 59B show ladder crossing in the CST model.

FIGS. 60A and 60B show footprint analysis in the CST model.

FIGS. 61A-61C show CGRP staining in spinal cord of (61A) LacZ, (61B) RARβ2 and (61C) GDNF transduced animals in the cervical rhizotomy model.

FIGS. 62A-62C show pERK labelling in the spinal cords of (62A) LacZ, (62B) RARβ2 and (62C) GDNF treated animals in the cervical rhizotomy model.

FIGS. 63A-63I show double immunohistochemistry with anti-β-galactosidase (β-gal; 63B, 63E, 63H) antibody in combination with either anti-neurofilament (N52; 63A), anti-CGRP (63D) or IB₄ (63G) antibodies. Merged images are shown in 63C, 63F and 63I.

FIG. 64 shows the percentage of β-gal neurones immunoreactive for neurofilament, CGRP or IB₄.

FIG. 65 shows the estimated biological titres of unconcentrated preparations made from pONY8.7-CORARβ2 and pseudotyped with the VSV-G envelope.

FIGS. 66A and 66B show relative amounts of RARβ2 and GNDF expression in transduced and uninjured/injured spinal cord tissue.

### DETAILED DESCRIPTION OF THE INVENTION

The present invention is advantageous because RARβ2 and/or an agonist thereof can cause modulation of neural cell development. It is also an advantage of the present invention that administration of NGF to a subject is avoided. It is a further advantage of the present invention that it enables neurite outgrowth and/or neurite regeneration to be promoted in adult neural tissue. Another advantage of the present invention is that it enables RARβ2 to be introduced into non-dividing mammalian cells such as neuronal cells. It is also an advantage of the present invention that the receptor may be delivered to cells whose environment comprises endogenous levels of agonist of the receptor, such as retinoic acid (RA).

Retinoids

Retinoids are a family of molecules derived from vitamin A and include the biologically active metabolite, retinoic acid (RA). The cellular effects of RA are mediated through the action of two classes of receptors, the retinoic acid receptors (RARs) which are activated both by all-trans-RA (tRA) and 9-cis-RA (9-cis-RA), and the retinoid X receptors (RXRs), which are activated only by 9-cis-RA (Kastner et al., 1994; Kleiwer et al., 1994). The receptors are of three major subtypes, α, β and γ, of which there are multiple isoforms due to alternative splicing and differential promoter usage (Leid et al.). The RARs mediate gene expression by forming heterodimers with the RXRs, whilst the RXRs can mediate gene expression as homodimers or by forming heterodimers with a variety of orphan receptors (Mangelsdorf & Evans, 1995). Many studies on a variety of embryonic neuronal types have shown that RA can stimulate both neurite number and length (review, Maden, 1998), as, indeed, can the neurotrophins (Campenot, 1977; Lindsay, 1988; Tuttle and Mathew, 1995). The neurotrophins are a family of growth factors that are required for the survival of a variety of neurons of primary sensory neurons in the developing peripheral nervous system (Snider, 1994). One of the earliest genes induced by NGF in PC12 cells is the orphan receptor NGFI-B (NURR1) (Millbrandt, 1989). This suggests that the growth factor and retinoid mediated pathway in developing neurons can interact.

Background teachings on these aspects have been presented by Victor A. McKusick et al., for example on the ncbi website maintained by the NIH. The following information has been extracted from that source.

Three retinoic acid receptors, alpha, beta, and gamma, are members of the nuclear receptor superfamily. Retinoic acid was the first morphogen described in vertebrates. The RARA and RARB genes are more homologous to those of the 2 closely related thyroid hormone receptors THRA and THRB, located on chromosomes 17 and 3, respectively, than to any other members of the nuclear receptor family. These observations suggest that the thyroid hormone and retinoic acid receptors evolved by gene, and possibly chromosome, duplications from a common ancestor which itself diverged rather early in evolution from the common ancestor of the steroid receptor group of the family. The RARB gene, formerly symbolized HAP, maps to 3p24 by somatic cell hybridization and in situ hybridization.

Benbrook et al. (1988) showed a predominant distribution in epithelial tissues and therefore used the designation RAR (epsilon). By in situ hybridization, Mattei et al. (1988) assigned the RARB gene to 3p24. Using deletion mapping, de The et al. (1990) identified a 27-bp fragment located 59-bp upstream of the transcriptional start, which confers retinoic acid responsiveness on the herpesvirus thymidine kinase promoter. They found indications that both alpha and beta receptors act through the same DNA sequence. Mattei et al. (1991) assigned the corresponding gene to chromosome 14, band A, in the mouse, and to chromosome 15 in the rat.

Nadeau et al. (1992) confirmed assignment of the mouse homolog to the centromeric portion of chromosome 14.

From a comparison of a hepatitis-B virus (HBV) integration site present in a particular human hepatocellular carcinoma (HCC) with the corresponding unoccupied site in the nontumorous tissue of the same liver, Dejean et al. (1986) found that HBV integration placed the viral sequence next to a liver cell sequence that bears a striking resemblance to both an oncogene, ERBA, and the supposed DNA-binding domain of the human glucocorticoid receptor and estrogen receptor genes.

Dejean et al. (1986) suggested that this gene, usually silent or transcribed at a very low level in normal hepatocytes, becomes inappropriately expressed as a consequence of HBV integration, thus contributing to the cell transformation.

By means of a panel of rodent-human somatic cell hybrid DNAs, Dejean et al. (1986) localized the gene to chromosome 3. Further studies by de The et al. (1987) suggested that the HAP gene product may be a novel ligand-responsive regulatory protein whose inappropriate expression in liver is related to hepatocellular carcinogenesis. Brand et al. (1988) showed that the novel protein called HAP (for HBV-activated protein) is a retinoic acid receptor. They referred to this receptor as the beta type (RARB) and mapped it to 3p25-p21.

Lotan et al. (1995) found that the expression of RARB mRNA is selectively lost in premalignant oral lesions and can be restored by treatment with isotretinoin. Restoration of the expression of RARB mRNA was associated with a clinical response.

RARB, RARG, RXRB, and RXRG are expressed in the striatum. To study the effect of these genes on locomotion, Kreczel et al. (1998) developed single and double knockout mice and analyzed their locomotor skills by open field and rotarod testing. RARB-RXRB, RARB-RXRG, and RXRB-RXRG double null mutant mice, but not the corresponding single null mutants, exhibited reductions in forward locomotion when compared with wildtype littermates. Forty percent of the RARB-RXRB null mutants showed backward locomotion.

Rotarod test performance was impaired for RARB, RARB-RXRB, RARB-RXRG, and RXRB-RXRG mice. In contrast, RARA, RARG, RARA-RXRG, and RARG-RXRG null mice showed no defects in locomotion, even though both RARA and RARG are also expressed in the striatum. The morphology, development, and function of skeletal muscle, peripheral nerves, and spinal cord were normal in all single and double null mutants, as were balance reflexes. These results suggested to Kreczel et al. (1998) that RARB, RXRB, and RXRG are involved specifically in the control of locomotor behaviors, and that heterodimers of RARB with either RXRB or RXRG are the functional receptor units, such that RXRB and RXRG are functionally redundant.

Kreczel et al. (1998) studied the expression of D1 and D2 dopamine receptors (DIR and D2R), the most abundant dopamine receptors in the striatum, in these mutant mice. RARB-RXRB, RARB-RXRG, and RXRB-RXRG double null mutants, but not RARB or RXRG single mutants, exhibited 40% and 30% reduction in whole-striatal DIR and D2R transcripts, respectively, when compared with wildtype controls.

The reduction was mostly in the medioventral regions of the striatum, including the shell and core of the nucleus accumbens, and the mediodorsal part of the caudate putamen. The reduction was not due to loss of D2R-expressing neurons; no increase in apoptosis was noted. The histology of the striatum was normal.

The characterization of a retinoic acid response element in the D2R promoter by Samad et al. (1997) led Kreczel et al. (1998) to suggest that the reduction in D2R and D2R expression occurs on a transcriptional level. The RARB-RXRB, RARB-RXRG, and RXRB-RXRG double null mutants did not exhibit the normal increase in locomotion induced by cocaine, mimicking the phenotype of D1R-null mice.

Taken together, these results indicated to Kreczel et al. (1998) that retinoids are involved in controlling the function of the dopaminergic mesolimbic pathway and suggested that defects in retinoic acid signaling may contribute to neurological disorders.

Agonists

The agonist of the present invention may be any suitable RARβ2 agonist. Preferably, said agonist of RARβ2 is capable of activating RARβ2 in a transactivation assay.

The agonist may be an organic compound or other chemical. The agonist can be an amino acid sequence or a chemical derivative thereof, or a combination thereof. The agent may even be a nucleotide sequence—which may be a sense sequence or an anti-sense sequence. The agent may even be an antibody.

Typically, the agonist will be an organic compound. Typically the organic compound will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

For some applications, preferably the agent comprises at least one cyclic group. The cyclic group may be a polycyclic group, such as a non-fused polycyclic group. For some applications, the agonist comprises at least the one of said cyclic groups linked to another hydrocarbyl group.

Specific Agonists

An example of a specific agonist according to the present invention is retinoic acid (RA). Both common forms of retinoic acid (either all-trans retinoic acid (tRA), or 9-cis-RA) are agonists of RARβ2.

CD2019 is a RARβ2 agonist having the structure as discussed herein and as shown in (Elmazar et al., (1996) Teratology vol. 53 pp158-167). This and other agonists are also discussed in (Beard and Chandraratna p.194; Johnson et al., 1996). The structure of CD2019 is presented as Formula I in the attached figures.

An alternative RARβ2 agonist is presented as Formula II in the attached figures.

The present invention also encompasses mimetics or bioisosteres of the formulae of Formula I and/or Formula II.

Preferably the agonist useful according to the present invention is selective for RARβ2.

Assay to Determine RARβ2 Agonism

Examples of agonists according to the present invention may be identified and/or verified by using an assay to determine RARβ2 agonism.

Hence, the present invention also encompasses (i) detemining if a candidate agent is capable of acting as a RARβ2 agonist; (ii) if said candidate agent is capable of acting as a RARβ2 agonist then delivering said agent to a subject and in such an amount to cause neurite development.

Assay

Any one or more of appropriate targets—such as an amino acid sequence and/or nucleotide sequence—may be used for identifying an agent capable of modulating RAR/β2 in any of a variety of drug screening techniques. The target employed in such a test may be free in solution, affixed to a solid support, borne on a cell surface, or located intracellularly. The abolition of target activity or the formation of binding complexes between the target and the agent being tested may be measured.

The assay of the present invention may be a screen, whereby a number of agents are tested. In one aspect, the assay method of the present invention is a high through put screen.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO-A-84/03564.

It is expected that the assay methods of the present invention will be suitable for both small and large-scale screening of test compounds as well as in quantitative assays.

In one preferred aspect, the present invention relates to a method of identifying agents that selectively modulate RARβ2.

In a preferred aspect, the assay of the present invention utilises cells that display RARβ2 on their surface. These cells may be isolated from a subject possessing such cells. However, preferably, the cells are prepared by transfecting cells so that upon transfect those cells display on their surface RARβ2.

Another example of an assay that may be used is described in WO-A-9849271, which concerns an immortalised human terato-carcinoma CNS neuronal cell line, which is said to have a high level of neuronal differentiation and is useful in detecting compounds which bind to RARβ2.

In another aspect, the invention relates to the use of a vector capable of directing the expression of RARβ2 to produce cell(s) for use in agonist/antagonist assays. For example, in another aspect, the invention relates to an assay comprising neuronal cell(s), said cells comprising an EIAV-derived vector capable of directing the expression of RARβ2 in said cell(s).

Reporters

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Other protocols include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes may even be used. These and other assays are described, among other places, in Hampton R et al (1990, Serological Methods, A Laboratory Manual, APS Press, St Paul Minn.) and Maddox DE et al (1983, J Exp Med 15 8:1211).

Examples of reporter molecules include but are not limited to (galactosidase, invertase, green fluorescent protein, luciferase, chloramphenicol, acetyltransferase, (glucuronidase, exo-glucanase and glucoamylase. Alternatively, radiolabelled or fluorescent tag-labelled nucleotides can be incorporated into nascent transcripts which are then identified when bound to oligonucleotide probes.

By way of further examples, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Differential Expression Screening Techniques

Genes encode gene products, mainly polypeptides but also RNAs, that are involved in a huge variety of cellular processes. The technique of differential expression screening is based on the idea that by comparing expression under two sets of conditions, genes whose expression varies between those two conditions can be identified and their function related back to the differences between those conditions. For example, genes involved in a pathway responsive to mitogens such as plate-derived growth factor (PDGF) can be identified by comparing gene expression in cells exposed to PDGF versus gene expression in cells not exposed to PDGF. Thus the term “differential expression screening” as used herein means comparing gene expression between two cells under different conditions or two different cells under the same or different conditions, with the aim of identifying gene products that differ in their levels of expression between the two cells.

The differences in gene expression may be measured using a variety of techniques. The first main type of technique is based on the measurement of nucleic acids and is termed herein as “genomic or cDNA techniques”. A useful review is provided in Kozian and Kirschbaum (1999). The second main type of technique is based on the measurement of cellular protein content and is termed herein as “proteomic techniques”.

Genomic or cDNA Techniques

One method well known in the art is subtractive cDNA hybridisation. This technique involves hybridising a population of mRNAs from one cell (e.g. a control cell) with a population of cDNAs made from the mRNA of another cell (e.g. a cell exposed to PDGF). This step will remove all sequences from the cDNA preparation that are common to both cells. The cDNAs derived from mRNAs whose expression is upregulated in the cell exposed to PDGF will not have a corresponding mRNA from the control with which to hybridise and can be isolated. Typically, the cDNAs are also hybridised with mRNA from the same cell to confirm that they represent coding sequences. This procedure is described in detail in WO90/11361 where mRNA from cells from the roots of plants treated with a chemical, N-(amincarbonyl)-2-chlorobenzenesulphonamide, were used to produce a cDNA library that was then hybridised with mRNA from untreated root cells. The procedure identified a number of genes whose expression was upregulated by the chemical.

The polymerase chain reaction (PCR) has led to the development of a number of other methods. RT-PCR differential display was first described by Liang and Pardee (1992). This technique involves the use of oligo-dT primers and random 5′ oligonucleotide 10-mers to carry out PCR on reverse-transcribed RNA from different cell populations. PCR is often carried out using a radiolabelled nucleotide so that the products can be visualised after gel electrophoresis and autoradiography. Wilkinson et al. (1995) used PCR differential display to identify five mRNAs that are upregulated in strawberry fruit during ripening. A review of differential display RT-PCR (also known as differential display of mRNA) is provided in Zhang et al. (1998) and a recent improvement using ‘long distance’ PCR is described in Zhao et al. (1999).

Another technique is termed cDNA library screening. A review of this technique and the other two differential expression screening techniques mentioned above is provided in Maser and Calvet (1995).

Differential display competitive PCR is a fairly recent innovation that has been successfully used to study changes in global gene expression in situations where only a few genes change expression levels, such as exposure of MCF17 cell to oestradiol, and in more complex situations such as neuronal differentiation of human NTERA2 cells (Jorgensen et al., 1999).

A further PCR based technique is representational difference analysis (RDA)—see Kozian and Kirschbaum (1999) for review and references therein. Also reviewed in see Kozian and Kirschbaum (1999) is a technique termed serial analysis of gene expression.

The actual identification of gene products whose expression differs between the two cell populations can be carried out in a number of ways. Subtractive methods will inherently identify gene products whose expression differs since gene products whose expression is the same are eliminated from the sample. Other methods include simply comparing the expression products from one cell with the expression products from another and looking for any differences (with PCR-based techniques, the number of products in each sample can be limited to a reasonable size), optionally with the aid of a computer program. For example using a PCR-based technique a visual comparison of bands present in different lanes allows the identification of bands unique to one lane. These bands can be cut out of the gel and subsequently analysed.

The advent of DNA chip technology, allows comparisons to be conveniently conducted by the use of microarrays (see Kozian and Kirschbaum, 1999 for review and references therein). Typically, arrays are generated using cDNAs (including ESTs), PCR products, cloned DNA and synthetic oligonucleotides that are fixed to a substrate such as nylon filters, glass slides or silicon chips. To determine differences in gene expression, labelled cDNAs or PCR products are hybridised to the array and the hybridisation patterns compared. The use of fluorescently labelled probes allows two different cell populations to be applied simultaneously to one chip and the results measured at different wavelengths A microarray-based differential expression screening technique is described in U.S. Pat. No. 5,800,992.

Proteomic Techniques

Proteomics is the study of proteins properties on a large scale to obtain a global, integrated view of disease processes, cellular processes and networks at the protein level. A review of techniques used in proteomics is given in Blackstock and Weir (1999)—see also references provided therein. The methods of the present invention are mainly concerned with expression proteomics, the study of global changes in protein expression in cells using electrophoretic techniques and image analysis to resolve proteins. Whereas nucleic acid analysis emphasises the message, proteomics is more concerned with the product. The two approaches are sometimes complementary since proteomic techniques may be useful in detecting changes in polypeptide levels due to changes in protein stability rather than mRNA levels.

A well known and ubiquitous technique used in the field of proteomics involves measuring the polypeptide content of a cell using 2D polyacrylamide gel electrophoresis (PAGE) and comparing this with the polypeptide content of another cell. The results of electrophoresis are typically a gel visualised with a dye such as silver stain or Coomassie-blue, or an autoradiograph produced from the gel, all with spots corresponding to individual proteins. Fluorescent dyes are also available.

The aim is therefore to identify spots that differ between the two gels/autoradiographs, i.e. missing from one, reduced in intensity or increased in intensity. Thus in the case of proteomics, comparing gene expression simply involves comparing the protein profile from one cell with the protein profile from another. Commercial software packages are available for automated spot detection.

Spots of interest may be excised from gels and the proteins identified using techniques such as matrix-assisted-laser-desorption-ionisation-time-of-flight (MALDI-TOF) mass spectrometry and electrospray.

It may be desirable to perform some measure of prefractionation, such as centrifugation or free-flow electrophoresis to improve the identification of low abundance proteins. Special procedures have also been developed for basic proteins, membrane proteins and other poorly soluble proteins (Rabilloud et al., 1997).

The above discussion provides a description of prior art methods available to the skilled person for performing differential expression screening of two or more cell populations in a general sense. However, the present invention is distinguished from these prior art methods in that a further step is required, namely that the levels of an endogenous biological molecule in a cell are altered by the experimenter, so that the levels of gene products that are affected by the molecule become more responsive to cellular perturbations such as signalling events. In other words, the object is to amplify and/or increase the signal to noise ratio of the differential response normally obtained so as to increase the likelihood of detecting gene products whose levels in a cell are low and/or whose expression normally changes by only a small amount.

By way of an example, the transcription factor HIF-1α is responsive to intracellular oxygen levels. Decreases in oxygen levels increase HIF-1α activity and lead to increased transcription from genes comprising a hypoxia responsive element (HRE). If the levels of HIF-1α in the cell are raised artificially, for example by infecting cells with a viral vector that directs expression of HIF-1α, then you would expect to see an increase in the transcriptional response mediated by HIF-1α. Consequently, changes in the expression of genes whose expression is sensitive to the HIF-1α mediated hypoxic response should be greater than in normal cells expressing physiological levels of HIF-1α.

Biological Molecules

The biological molecule can be any compound that is found in cells as a result of anabolic or catabolic processes within a cell or as a result of uptake from the extracellular environment, by whatever means. The term “biological molecule” means that the molecule has activity in a biological sense. Preferably the biological molecule is synthesised within the cell, i.e. is endogenous to that cell, or in the case of multicellular organisms, also within any of the cells of the organism.

Examples of biological molecules will therefore include proteins, nucleic acids, carbohydrates, lipids, steroids, co-factors, prosthetic groups (such as haem), inorganic molecules, ions (such as Ca²⁺), inositides. Where appropriate, precursors, monomeric, oligomeric and polymeric forms, and breakdown products of the above are also included.

Example of polypeptides include enzymes, transcription factors, hormones, structural components of cells and receptors including membrane bound receptors.

Preferably, the biological molecule is known to be involved in the cellular process of interest.

In one embodiment of the invention, the biological molecule is responsive to a signal, which may be an externally applied signal such as an environmental signal, for example redox stress, the binding of an extracellular ligand to a cell surface receptor leading to a cellular response mediated by a signal transduction signal. Alternatively, the signal may be an internally applied signal such as an increase in kinase activity due to falling levels of a cell metabolite.

The levels of the biological molecule may be altered directly or indirectly. Direct alteration may be achieved by, for example, causing cells to take up the molecule by incubating cells in a medium containing higher than physiological levels of the molecule. Other methods include vesicle-mediated delivery and microinjection. In the case of nucleic acids and polypeptides, the level of the biological molecule in the cell may be raised by the introduction of a heterologous nucleic acid into the cell which directs the expression of the nucleic acid or polypeptide.

The term “heterologous nucleic acid” in the present context means that the nucleic acid is not present in its natural context i.e. the cell has been modified so as to contain the nucleic acid which would otherwise not be present in the form in which it is introduced. For example, the nucleic acid may be extrachromosomal. The nucleic acid may also be integrated into the genome by viral transduction or homologous recombination. Nonetheless, part of all of the heterologous nucleic may be identical to a corresponding genomic sequence since the introduction of additional copies of a gene is a convenient means for increasing the levels of expression of that gene.

Indirect means for altering the levels of the biological molecule are numerous and include increasing the levels of an inhibitory or stimulatory molecule using the methods described above. Inhibitory molecules include antisense nucleic acids, ribozyme or an EGS directed against the mRNA encoding the biological molecule, a transdominant negative mutant directed against the biological molecule, transcription factors, enzyme inhibitors, and intracellular antibodies such as scFvs. Stimulatory molecules include enzyme activators, transcriptional activators. Thus cells may be manipulated in a number of ways such that ultimately the levels of the biological molecule are altered. Reduced expression may be achieved by expressing an anti-sense RNA,

The levels of the biological molecule are altered relative to physiological levels. Thus they may be enhanced or reduced. The term “relative to physiological levels” means relative to the concentration or activity of the biological molecule typically present in the cell under normal physiological conditions prior to manipulation of those levels. Thus the intention is that by deliberate means, the activity of the biological molecule is altered above or below that which is found in the cell under a range of normal physiological conditions. “Physiological conditions” includes the conditions normally found in vivo and the conditions normally used in vitro to culture the cells.

By way of an example, the activity or concentration may be increase or decreased 5-fold, 10-fold, 20-fold, 50-fold or 100-fold compared to the normal physiological activity or concentration found in the cell prior to introducing, for example, the heterologous nucleic acid.

Where, as in a preferred embodiment of the invention, the levels of the biological molecule are altered by the introduction of a heterologous nucleic acid, typically a nucleic acid that directs expression of a polypeptide, the heterologous nucleic acid will comprise a coding sequences operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Control sequences operably linked to sequences encoding the protein of the invention include promoters/enhancers and other expression regulation signals. These control sequences may be selected to be compatible with the host cell in which the expression vector is designed to be used. The term promoter is well known in the art and encompasses nucleic acid regions ranging in size and complexity from minimal promoters to promoters including upstream elements and enhancers.

The promoter is typically selected from promoters which are functional in mammalian, cells, although promoters functional in prokaryotic cells or other eukaryotic cells may be used where appropriate. Thus, the promoter is typically derived from promoter sequences of viral or eukaryotic genes. For example, it may be a promoter derived from the genome of a cell in which expression is to occur. Eukaryotic promoters, may be promoters that function in a ubiquitous manner (such as promoters of α-actin, β-actin, tubulin) or, alternatively, a tissue-specific manner (such as promoters of the genes for pyruvate kinase). Tissue-specific promoters specific for particular cells may be used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may be advantageous for the promoters to be inducible so that the levels of expression from the heterologous nucleic acid can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Suitable vectors include plasmids, artificial chromosomes and viral vectors. Viral vectors include DNA virus vectors, RNA virus vectors (ie. retroviral vectors), such as lentiviruses, adenoviral vectors, adeno-associated vectors and herpes simplex viral vectors. Vectors/polynucleotides may introduced into suitable host cells using a variety of techniques known in the art, such as transfection, transformation, electroporation, infection with recombinant viral vectors such as retroviruses, herpes simplex viruses and adenoviruses, direct injection of nucleic acids and biolistic transformation. It is particularly preferred to use recombinant viral vector-mediated techniques.

A cell of interest can be any cell, for example a prokaryotic cell, a yeast cell, a plant cell or an animal cell, such as an insect cell or a mammalian cell, including a human cell. In the case of cells from multicellular organism, cells may be primary cells or immortalised cell lines. Although cells are frequently referred to in the singular, in general cells will be part of a cell population.

In certain aspects of the invention, a comparison is required between gene expression in at least two distinct cells. Typically the first of the two or more cells is termed a reference cell. In a preferred embodiment, the cells to be used in the comparison are substantially identical in all respects. For example they may both be cells of the same cell line or obtained from the same tissue in an organism. One or both of the cells may then be manipulated so that they comprise altered levels, relative to physiological levels, of the biological molecule as described above. In one embodiment, the first cell is unaltered and the second cell altered. This is particularly preferred since it should result in an improved signal to noise ratio. In a highly preferred embodiment, the first cell is unaltered, and the second cell comprises RARβ2 according to the present invention. Preferably, the cells are mammalian neuronal cells.

Nonetheless, it is not necessary that the cells used as the starting point of the investigation be substantially identical. For example, in one aspect of the invention, genes involved in disease processes may be investigated using cells from a diseased organism, such as a mammalian patient. These may be compared with cells from a normal organism or similar cells from the same or a different diseased individual. Where cells from a normal organism and a diseased organism are used, generally the normal cells correspond to the first cell of interest and the diseased cells correspond to the second cell of interest. Consequently, at least the diseased cells are modified as described above in so that comprises altered levels of the biological molecule.

In another embodiment, one cell is a cell comprising a mutant gene whereas the other cell comprises a wild-type version of the same gene.

Another possibility is that the cells are from different tissues or from different stages in development or differentiation, for example as affected by the presence or absence of RARβ2, and/or retinoic acid or derivatives thereof.

The present invention provides a number of improved methods for identifying genes by differential expression screening techniques.

In another aspect, a method is provided for identifying genes involved in a cellular process.

Essentially one of the cells is manipulated so that the levels within that cell of a biological molecule involved in the cellular process are altered. Preferably, this process is neurite outgrowth and/or neural regeneration as effected by the action of retinoic acid through RARβ2. Typically, this is achieved by the introduction of a heterologous nucleic acid into the cell to direct the expression of a polypeptide such as RARβ2. The polypeptide may be the same as the biological molecule or it may modulate the levels of the biological molecule as described above.

In general, simply modulating the levels of a biological molecule in one of two identical cells and then measuring gene transcription is not the aim of the methods of the present invention since you will be measuring the effect of the biological molecule on gene expression in the cells rather than using the change in the levels of the biological molecule to enhance or reduce the response to an event of interest.

However, where the biological molecule is a gene product, such as a polypeptide, that is produced naturally within the cell, altering the levels of the gene product by the introduction of a heterologous nucleic acid may be used to simultaneously both perturb a cellular process and enhance the response to such a perturbation making it easier to identify gene products involved in that cellular process using differential expression techniques. By way of an example, overexpression of HIF-1α not only induces an hypoxic response but amplifies the downstream elements of that response due an enhanced regulatory effect on HIF-1α mediated transcription.

Nonetheless in the broader aspects of the present invention, two main possibilities arise. Firstly, the two cells are different and have inherently different gene expression patterns. In this situation, alterations in the levels of the biological molecule can be used to enhance those differences. The two cells may be, for example, from different tissues, or from different stages in development or differentiation. The two cells may also be different by virtue of one cell being from diseased tissue and the other cell from normal tissue. Other configurations envisaged are given above.

Secondly, the two cells are the same but one of the cells is stimulated in some manner and the other cell not (or one is stimulated to a greater extent than the other). For example, one cell is incubated in the presence of a growth factor and the other is not. The growth factor is therefore not the biological molecule but is instead a stimulus designed to perturb gene expression in the cell, the effects of which may be amplified by the biological molecule which in turn is altered in level by the polypeptide expressed from the heterologous nucleic acid.

Thus in a second aspect there is provided a method whereby genes whose expression is regulated by a signal are identified by subjecting two distinct cell populations to different levels of a signal, whereby either or both cells have been manipulated so as to alter the levels of a biological molecule whose activity is responsive to the signal, and identifying gene products whose expression differs. The term “whose activity is response to the signal” includes biological molecule whose concentration in the cell varies in response to the signal as well as biological molecules whose properties such as enzymatic activity or affinity for another cellular component varies in response to the signal.

Thus returning to our factor example, the cells that are exposed to the factor may have been altered to express increased levels of a transcription factor involved in the signal transduction cascade. Consequently, the effect of the growth factor will be increased downstream of the transcription factor (in either a negative or positive sense) making it easier to identify differentially expressed genes whose expression is regulated by the transcription factor and ultimately by the factor. Preferably the factor leads to stimulation of neural regeneration/neurite outgrowth via signalling through RARβ2.

The signal may be either physical, such as redox conditions, CO₂ levels, light or temperature, or chemical such as ligands that bind to receptors on the cell surface and trigger signal transduction pathways (including hormones or cell surface molecules normally attached to other cells), or substrates for enzyme reactions that diffuse into or are transported into the cell.

The first cell is subjected the signal at a first level and the second cell is subjected to the signal at a second level. The first level may simply be the absence of the signal and the second level may be the presence of the signal, or vice-versa. The levels of the signals may be adjusted so as to provide a discernible difference in gene expression but are preferably at physiologically relevant levels.

In another aspect of the present invention, knowledge already acquired about genes involved in a disease or other biological process may be used to generate further information about other genes whose expression is altered in a disease or other biological process. To do this, one cell is modified so that the levels of the gene product known to be involved in the disease or other biological process are altered, either directly by the introduction of a heterologous nucleic acid encoding the gene product, or indirectly as described above. Gene expression is then measured in both cells and the results compared to identify gene products whose expression varies.

In this aspect of the invention, the two cells may be identical, except for the change in the levels of the gene product known to be involved in the disease or other biological process of interest. The two cells may thus both be normal cells of the same type as a cell type in which the disease or other process manifests itself, or they may both be diseased cells. Alternatively, one cell may be normal and the other diseased. Preferably the diseased cell is the modified cell if only one of the cells is modified.

In another aspect of the invention, differential expression screening methods are used to identify genes involved in a disease or other process in a two stage procedure. Firstly, gene expression is compared between a first cell of interest, for example a cell from a normal patient, and a second cell of interest, for example corresponding cells from a diseased patient. As discussed above, the first cell and the second cell will be different in some aspect such that they have different expression patterns. This may be because the cells are from different tissues or different individuals (for example a normal patient and a diseased patient) or the cells may be of similar origin but have been treated differently in some respect.

Gene products whose expression differs between the first cell and the second cell are identified. Secondly, a third cell of interest, essentially identical to the first cell is used in a screening procedure where a candidate gene is introduced into the third cell so that levels of the genes are altered (typically raised). Gene expression in this cell is compared with gene expression in the first cell and gene products whose expression differs between the normal cell and the third cell comprising altered levels of the candidate gene are identified. If a gene product whose expression is altered in the second cell also has altered gene expression in the third cell, then the candidate gene is selected for further study. Preferably there is a correlation over two or more gene products, preferably at least four or five gene products to minimise false positives.

Clearly, the methods of the present invention may advantageously be applied to the differential analysis of non-dividing neuronal cells and a different sample of the same cells which have been induced to regenerate or undergo neurite outgrowth by the methods of the present invention. This differential analysis applies to the discovery and/or validation of candidate molecules, in particular those biological molecules which lie in the signalling pathway between the activation of the RARβ2 receptor and the actual morphological phenotype of neurite outgrowth and/or neurite regeneration. This phenomenon of neurite outgrowth/regeneration will be brought about by physiological changes within the cell which are initiated by the activation of RARβ2, and may include changes in gene expresison. Thus, by taking a sample of neuronal cells and introducing RARβ2 as described herein, and allowing retinoic acid to signal through this receptor, and comparing the pattern of gene expression with a sample of such cells which do not contain RARβ2/retinoic acid, key difference(s) in gene expression may be identified. The pathway(s) leading to neurite outgrowth will be switched on in the cells with RARβ2/retinoic acid. By making cDNA from these and from the non-activated cells in parallel, subtractive cDNA libraries may be made in order to isolate differences in gene expression between the two sets of cells. This or other differential screening technique(s), or proteomic techniques such as 2-D electrophoretic mapping, can be used to detect the stimulation and/or repression of particular gene(s) or sets of genes which the different conditions produce. These differentially expressed genes and/or their gene products are each individual candidate factors in the stimulation of neurite outgrowth, and it will be clearly understood that the invention relates also to these. This topic is discussed in more detail below.

Host or Target Cells

Polynucleotides for use in the present invention—such as for use as targets or for expressing targets or for use as the pharmaceutically active agent—may be introduced into host cells.

The term “host cell” or “target cell”—in relation to the present invention includes any cell that could comprise the polynucleotide sequence of the present invention.

Here, polynucleotides may be introduced into prokaryotic cells or eukaryotic cells, for example yeast, insect or mammalian cells.

Polynucleotides of the invention may introduced into suitable host cells using a variety of techniques known in the art, such as transfection, transformation and electroporation. Where polynucleotides of the invention are to be administered to animals, several techniques are known in the art, for example infection with recombinant viral vectors such as retroviruses, herpes simplex viruses, adenoviruses, adeno-associated viruses, direct injection of nucleic acids and biolistic transformation. The selection of the particular technique for the administration of polynucleotides into particular host cell(s) is well within the abilities of a person skilled in the art and is further discussed herein. For example, a person wishing to administer polynucleotide to a non-dividing mammalian cell such as a neuronal cell would select a vector system capable or transfecting/transducing non-dividing mammalian cells. An example of such a vector is a viral vector such as a vector based on or derived from EIAV. This and further examples are discussed at length herein.

Thus, a further embodiment of the present invention provides host cells transformed or transfected with a polynucleotide that is or expresses the target of the present invention. Preferably said polynucleotide is carried in a vector for the replication and expression of polynucleotides that are to be the target or are to express the target. The cells will be chosen to be compatible with the said vector and may for example be prokaryotic (for example bacterial), fungal, yeast or plant cells.

The gram negative bacterium E. coli is widely used as a host for heterologous gene expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide encoding the polypeptide of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus oryzae, Trichoderma reesei, Bacillus subtilis, Bacillus licheniformis, Bacillus amyloliquefaciens, Kluyveromyces lactis and Saccharomyces cerevisiae.

Polypeptides that are extensively modified may require correct processing to complete their function. In those instances, mammalian cell expression systems (such as HEK-293, CHO, HeLA) are required, and the polypeptides are expressed either intracellularly, on the cell membranes, or secreted in the culture media if preceded by an appropriate leader sequence.

The use of suitable host cells—such as yeast, fungal, plant and mammalian host cells—may provide for post-translational modifications (e.g. myristoylation, glycosylation, truncation, lipidation and tyrosine, serine or threonine phosphorylation) as may be needed to confer optimal biological activity on recombinant expression products of the present invention.

Organism

The term “organism” in relation to the present invention includes any organism that could comprise the sequence according to the present invention and/or products obtained therefrom. Examples of organisms may include a fungus, yeast or a plant.

The term “transgenic organism” in relation to the present invention includes any organism that comprises the target according to the present invention and/or products obtained.

Transformation of Host Cells/Host Organisms

As indicated earlier, the host organism can be a prokaryotic or a eukaryotic organism.

Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, eg G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Further hosts suitable for the nucleotide sequence of the present invention include higher eukaryotic cells, such as insect cells or vertebrate cells, particularly mammalian cells, including human cells, or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells.

The nucleotide sequence of the present invention may be stably incorporated into host cells or may be transiently expressed using methods known in the art. By way of example, stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of the nucleotide sequence of the present invention. The precise amounts of the nucleotide sequence of the present invention may be empirically determined and optimised for a particular cell and assay.

Thus, the present invention also provides a method of transforming a host cell with a nucleotide sequence that is to be the target or is to express the target. Host cells transformed with the nucleotide sequence may be cultured under conditions suitable for the expression of the encoded protein. The protein produced by a recombinant cell may be displayed on the surface of the cell. If desired, and as will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

Receptors

The RARβ2 receptor as discussed herein includes mimetics, homologues, fragments and part or all of the entire gene product. Preferably the RARβ2 receptor as discussed herein refers to substantially the entire gene product.

In one embodiment, the present invention relates to the use of a receptor in the production of neurite outgrowth. Previously, attempts have been made to produce neurite outgrowth using a number of different techniques. Typically, nerve growth factor (NGF) is used to stimulate neurite outgrowth. However, NGF is a relatively large molecule with a correspondingly high molecular weight, and is susceptible to protease mediated degradation. NGF is also relatively expensive to prepare. Similar approaches to the stimulation of neurite outgrowth have also encountered various difficulties. Moreover, such approaches have centred on the use of stimulatory factors such as growth factors in order to produce such desired phenotype(s). However, it is surprisingly shown herein that the long-felt need for the production of neurite outgrowth, for example in non-dividing cells, may be achieved using the converse approach disclosed herein, ie. the use of receptors to stimulate neurite outgrowth as described and demonstrated in the present invention. This disclosure runs against current thinking in the art, which has been focussed on the use of growth factors to try to elicit neurite outgrowth from non-dividing cells such as terminally differentiated neuronal cells. The surprising finding that receptor(s) may be delivered to such cells to produce neural regeneration/neurite outgrowth is illustrated herein by using RARβ2 as an example of this general approach. Thus, the present invention relates to the use of a receptor in the production of neurite outgrowth. The receptor may be any eukaryotic receptor, preferably a vertebrate receptor, more preferably a mammalian receptor, more preferably a primate receptor, most preferably a human receptor. Receptors for use in the present invention may comprise one or more membrane-spanning domain(s). In a preferred embodiment, receptors useful in the present invention are human receptors, without regard to their natural temporal and/or spatial expression profile. In a highly preferred embodiment, receptors useful in the present invention are human receptors which are not normally expressed in cell(s) of the adult target tissue. In a most highly preferred embodiment, receptors useful in the present invention are retinoic acid receptors such as RARs, such as in particular RARβ2. Receptor(s) useful in the present invention are preferably delivered to the target cell(s) using a vector system as described herein, such as a lentiviral vector system.

Neurological Disorders

Clearly, stimulation of neurite outgrowth and/or neurite regeneration according to the present invention will have therapeutic benefit in a number of pathologies. These include, but are not limited to, neurological disorders, for example degenerate neurological disorders such as Parkinson's disease, Alzheimer's syndrome, or related conditions, or neural (nerve) injury such as spinal cord injury, avulsion injury or other such physical condition.

The term neurological disorders as used herein may refer to any injury, whether mechanically (for example by trauma, such as an avulsion injury) or chemically induced (for example by neurotoxin(s), or by an regime of treatment having an immunosuppressant effect, whether by design, or as a side-effect), any neural pathology such as caused by viral infection or otherwise, any degenerative disorder, or other nerve tissue related disorder.

Examples of neurological disorders include conditions such as Parkinson's disease, Alzheimer's disease, senility, motor neurone disease, schizophrenia as well as other neural and/or neurodegenerative disorders. Other neural related disorders may include glaucoma or other cause of damage to the optic nerve, Bell's palsy or other forms of localised paralysis, neurally based impotence such as caused by nerve trauma following radical prostatectomy, or other complaints. Other disorders in which the invention may be useful include neuropathological effects of diabetes, AIDS neuropathy, leprosy etc.

The term neurological disorder refers to any disorder of a nervous system, whether the peripheral nervous system or the central nervous system (CNS), whether the sympathetic nervous system, or the parasympathetic nervous system, or whether affecting a subset or superset of different nerve types.

Nucleotide of Interest (NOI)

In accordance with the present invention, the NOI sequence may encode a peptide which peptide may be the pharmaceutically active agent—such as an RA receptor, preferably RARβ2, or an agonist thereof.

Such coding NOI sequences may be typically operatively linked to a suitable promoter capable of driving expression of the peptide, such as in one or more specific cell types.

In addition to the NOI or part thereof and the expression regulatory elements described herein, the delivery system may contain additional genetic elements for the efficient or regulated expression of the gene or genes, including promoters/enhancers, translation initiation signals, internal ribosome entry sites (IRES), splicing and polyadenylation signals.

The NOI or NOIs may be under the expression control of an expression regulatory element, usually a promoter or a promoter and enhancer. The enhancer and/or promoter may be preferentially active in neural cells, such that the NOI is preferentially expressed in the particular cells of interest, such as in nerve cells. Thus any significant biological effect or deleterious effect of the NOI on the individual being treated may be reduced or eliminated.

The enhancer element or other elements conferring regulated expression may be present in multiple copies. Likewise, or in addition, the enhancer and/or promoter may be preferentially active in one or more specific cell types—such as neural cells for example post-mitotically terminally differentiated non-replicating cells such as neurons.

The term “promoter” is used in the normal sense of the art, e.g. an RNA polymerase binding site in the Jacob-Monod theory of gene expression.

The term “enhancer” includes a DNA sequence which binds to other protein components of the transcription initiation complex and thus facilitates the initiation of transcription directed by its associated promoter.

Expression Vector

Preferably, the NOI (e.g. that encoding RARβ2 or part thereof) used in the method of the present invention is inserted into a vector which is operably linked to a control sequence that is capable of providing for the expression of the coding sequence by the host cell, i.e. the vector is an expression vector.

Codon Optimisation

As used herein, the terms “codon optimised” and “codon optimisation” refer to an improvement in codon usage. By way of example, alterations to the coding sequences for genes or viral components may improve the sequences for codon usage in the mammalian cells or other cells which are to act as the producer cells for retroviral vector particle production. This is referred to as “codon optimisation”. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Preferably a high titre lentiviral vector is produced using a codon optimised gag and a codon optimised pol or a codon optimised env (see seq. listing and/or WO99/41397).

Preferably a high titre retroviral vector is produced using a modified and/or extended packaging signal.

Preferably nucleic acid sequences and genes of interest are codon optimised. In a highly preferred embodiment, the nucleic acid sequence encoding RARβ2 is codon optimised

Packaging Signal

As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi sequence) needed for encapsidation of the viral genome. By way of example, this minimal packaging signal encompasses bases 212 to 563 of the Mo-MLV genome (Mann et al 1983: Cell 33: 153).

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Preferably a high titre lentiviral vector is produced using a modified packaging signal.

Preferably the lentiviral construct is a based on an EIAV vector genome where all the accessory genes are removed except Rev.

Accessory Genes

As used herein, the term “accessory genes” refer to a variety of virally encoded accessory proteins capable of modulating various aspects of retroviral replication and infectivity. The term “accessory genes” is interchangeable with the term “auxiliary genes.” These proteins are discussed in Coffin et al (ibid) (Chapters 6 and 7). Examples of accessory proteins in lentiviral vectors include but are not limited to tat, rev, nef, vpr, vpu, vif, vpx. An example of a lentiviral vector useful in the present invention is one which has all of the accessory genes removed except rev, i.e., an example of a miminal lentiviral vector.

Transcriptional Control

The control of proviral transcription remains largely with the noncoding sequences of the viral LTR. The site of transcription initiation is at the boundary between U3 and R in the left hand side LTR and the site of poly (A) addition (termination) is at the boundary between R and U5 in the right hand side LTR. The 3′U3 sequence contains most of the transcriptional control elements of the provirus, which include the promoter and multiple enhancer sequences responsive to cellular and in some cases, viral transcriptional activator proteins.

An LTR present, for example, in a construct of the present invention and as a 3′LTR in the provirus of, for example, a target cell of the invention may be a native LTR or a heterologous regulatable LTR. It may also be a transcriptionally quiescent LTR for use in SIN vector technology.

The term “regulated LTR” also includes an inactive LTR such that the resulting provirus in the target cell can not produce a packagable viral genome (self-inactivating (SIN) vector technology).

Preferably the regulated retroviral vector of the present invention is a self-inactivating (SIN) vector.

Self-Inactivating (SIN) Vector

By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al 1986 Proc Natl Acad Sci 83: 3194-3198; Dougherty and Temin 1987 Proc Natl Acad Sci 84: 1197-1201; Hawley et al 1987 Proc Natl Acad Sci 84: 2406-2410; Yee et al 1987 Proc Natl Acad Sci 91: 9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al 1983 Nucleic Acids Res 11: 1855-1872) or suppression of transcription (Emerman and Temin 1984 Cell 39: 449-467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Preferably the targeted vector has a pseudotyped envelope gene in order to effectively transduce a specific cell type.

Envelope (ENV)

If the retroviral component includes an env nucleotide sequence, then all or part of that sequence can be optionally replaced with all or part of another env nucleotide sequence such as, by way of example, the amphotropic Env protein designated 4070A or the influenza haemagglutinin (HA) or the vesicular stomatitis virus G (VSV-G) protein. Replacement of the env gene with a heterologous env gene is an example of a technique or strategy called pseudotyping. Examples of pseudotyping may be found in WO-A-98/05759, WO-A-98/05754, WO-A-97/17457, WO-A-96/09400, WO-A-91/00047 and Mebatsion et al 1997 Cell 90, 841-847.

In one preferred aspect, the retroviral vector of the present invention has been pseudotyped. In this regard, pseudotyping can confer one or more advantages. For example, with the lentiviral vectors, the env gene product of the HIV based vectors would restrict these vectors to infecting only cells that express a protein called CD4. But if the env gene in these vectors has been substituted with env sequences from other RNA viruses, then they may have a broader infectious spectrum (Verma and Somia 1997 Nature 389:239-242). By way of example, workers have pseudotyped an HIV based vector with the glycoprotein from VSV (Verma and Somia 1997 ibid).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al 1996 Gene Therapy 3: 280-6; Fielding et al 1998 Blood 9: 1802 and references cited therein).

The term “retroviral vector particle” refers to the packaged retroviral vector, that is preferably capable of binding to and entering target cells. The components of the particle, as already discussed for the vector, may be modified with respect to the wild type retrovirus. For example, the Env proteins in the proteinaceous coat of the particle may be genetically modified in order to alter their targeting specificity or achieve some other desired function.

Preferably, the viral vector preferentially transduces a certain cell type or cell types.

More preferably, the viral vector is a targeted vector, that is it has a tissue tropism which is altered compared to the native virus, so that the vector is targeted to particular cells.

For retroviral vectors, this may be achieved by modifying the Env protein. The Env protein of the retroviral secondary vector needs to be a non-toxic envelope or an envelope which may be produced in non-toxic amounts within the primary target cell, such as for example a MMLV amphotropic envelope or a modified amphotropic envelope. The safety feature in such a case is preferably the deletion of regions or sequence homology between retroviral components.

Preferably the envelope is one which allows transduction of human cells. Examples of suitable env genes include, but are not limited to, VSV-G, Rabies-G, a MLV amphotropic env such as the 4070A env, the RD114 feline leukaemia virus env or haemagglutinin (HA) from an influenza virus. The lentiviral vector pseudotyped with Rabies-G is advantageously as in WO99/61639 or in other patent documents (e.g., applications) incorporated herein by reference wherein the assignee or applicant (e.g., on PCT and UK applications) is Oxford Biomedica, which can also be sources for additional vectors or additional coding sequences to be employed in the practice of the invention.

The Env protein may be one which is capable of binding to a receptor on a limited number of human cell types and may be an engineered envelope containing targeting moieties. The env and gag-pol coding sequences are transcribed from a promoter and optionally an enhancer active in the chosen packaging cell line and the transcription unit is terminated by a polyadenylation signal. For example, if the packaging cell is a human cell, a suitable promoter-enhancer combination is that from the human cytomegalovirus major immediate early (hCMV-MIE) gene and a polyadenylation signal from SV40 virus may be used. Other suitable promoters and polyadenylation signals are known in the art.

The packaging cell may be an in vivo packaging cell in the body of an individual to be treated or it may be a cell cultured in vitro such as a tissue culture cell line. Suitable cell lines include mammalian cells such as murine fibroblast derived cell lines or human cell lines. Preferably the packaging cell line is a human cell line, such as for example: 293 cell line, HEK293, 293-T, TE671, HT1080.

Alternatively, the packaging cell may be a cell derived from the individual to be treated such as a monocyte, macrophage, stem cells, blood cell or fibroblast. The cell may be isolated from an individual and the packaging and vector components administered ex vivo followed by re-administration of the autologous packaging cells. Alternatively the packaging and vector components may be administered to the packaging cell in vivo. Methods for introducing retroviral packaging and vector components into cells of an individual are known in the art. For example, one approach is to introduce the different DNA sequences that are required to produce a retroviral vector particle e.g. the env coding sequence, the gag-pol coding sequence and the defective retroviral genome into the cell simultaneously by transient triple transfection (Landau & Littman 1992 J. Virol. 66, 5110; Soneoka et al 1995 Nucleic Acids Res 23:628-633). A four vector system may also be used, wherein the rev gene is introduced on a fourth cassette.

In one embodiment the vector configurations of the present invention use as their production system, three transcription units expressing a genome, the gag-pol components and an envelope. The envelope expression cassette may include one of a number of envelopes such as VSV-G, Rabies-G or various murine retrovirus envelopes such as 4070A. In addition, a fourth vector cassette may be included in the system if rev is utilized and is supplied on a separate cassette in trans.

Conventionally these three cassettes would be expressed from three plasmids transiently transfected into an appropriate cell line such as 293T or from integrated copies in a stable producer cell line.

Replication Vectors

The nucleotide sequences encoding the of the present invention may be incorporated into a recombinant replicable vector. The vector may be used to replicate the nucleotide sequence in a compatible host cell. Thus in one embodiment of the present invention, the invention provides a method of making the RARβ2 of the present invention by introducing a nucleotide sequence of the present invention into a replicable vector, introducing the vector into a compatible host cell, and growing the host cell under conditions which bring about replication of the vector. The vector may be recovered from the host cell.

Host/Target Cells

Host and/or target cells comprising nucleotide sequences of the present invention may be used to express the RARβ2 of the present invention under in vitro, in vivo and ex vivo conditions.

The term “host cell” and/or “target cell” includes any cell derivable from a suitable organism which a vector is capable of transfecting or transducing. Examples of host and/or target cells can include but are not limited to cells capable of expressing the RARβ2 of the present invention under in vitro, in vivo and ex vivo conditions. Examples of such cells include but are not limited to neuronal cells, nerve cells, post-mitotically terminally differentiated non-replicating cells such as neurons or combinations thereof.

In a preferred embodiment, the cell is a mammalian cell.

In a highly preferred embodiment, the cell is a human cell.

The term “organism” includes any suitable organism. In a preferred embodiment, the organism is a mammal. In a highly preferred embodiment, the organism is a human.

The present invention also provides a method comprising transforming a host and/or target cell with a or the nucleotide sequence(s) of the present invention.

The term “transformed cell” means a host cell and/or a target cell having a modified genetic structure. With the present invention, a cell has a modified genetic structure when a vector according to the present invention has been introduced into the cell.

Regulation of Expression In Vitro/Vivo/Ex Vivo

The present invention also encompasses gene therapy whereby the RARβ2 encoding nucleotide sequence(s) of the present invention is regulated in vitro/in vivo/ex vivo. For example, expression regulation may be accomplished by administering compounds that bind to the RARβ2 encoding nucleotide sequence(s) of the present invention, or control regions associated with the RARβ2 encoding nucleotide sequence of the present invention, or its corresponding RNA transcript to modify the rate of transcription or translation.

Control Sequences

Control sequences operably linked to sequences encoding the RARβ2 of the present invention include promoters/enhancers and other expression regulation signals. These control sequences may be selected to be compatible with the host cell and/or target cell in which the expression vector is designed to be used. The control sequences may be modified, for example by the addition of further transcriptional regulatory elements to make the level of transcription directed by the control sequences more responsive to transcriptional modulators.

Operably Linked

The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Preferably the nucleotide sequence of the present invention is operably linked to a transcription unit.

The term “transcription unit(s)” as described herein are regions of nucleic acid containing coding sequences and the signals for achieving expression of those coding sequences independently of any other coding sequences. Thus, each transcription unit generally comprises at least a promoter, an optional enhancer and a polyadenylation signal.

Promoters

The term promoter is well-known in the art and is used in the normal sense of the art, e.g. an RNA polymerase binding site. The term encompasses nucleic acid regions ranging in size and complexity from minimal promoters to promoters including upstream elements and enhancers.

The promoter is typically selected from promoters which are functional in mammalian, cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase).

Preferably the promoter is a constitutive promoter such as CMV.

Preferably the promoters of the present invention are tissue specific.

Tissue-Specific Promoters

The promoters of the present invention may be tissue-specific promoters. Examples of suitable tissue restricted promoters/enhancers are those which are highly active in tumour cells such as a promoter/enhancer from a MUCI gene, a CEA gene or a 5T4 antigen gene. Examples of temporally restricted promoters/enhancers are those which are responsive to ischaemia and/or hypoxia, such as hypoxia response elements or the promoter/enhancer of a grp78 or a grp94 gene. The alpha fetoprotein (AFP) promoter is also a tumour-specific promoter. One preferred promoter-enhancer combination is a human cytomegalovirus (hCMV) major immediate early (MIE) promoter/enhancer combination.

Preferably the promoters of the present invention are tissue specific. That is, they are capable of driving transcription of a RARβ2 encoding nucleotide sequence(s) in one tissue while remaining largely “silent” in other tissue types.

The term “tissue specific” means a promoter which is not restricted in activity to a single tissue type but which nevertheless shows selectivity in that they may be active in one group of tissues and less active or silent in another group.

The level of expression of a or the RARβ2 encoding nucleotide sequence(s) under the control of a particular promoter may be modulated by manipulating the promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity.

A number of tissue specific promoters, described above, may be particularly advantageous in practising the present invention. In most instances, these promoters may be isolated as convenient restriction digestion fragments suitable for cloning in a selected vector. Alternatively, promoter fragments may be isolated using the polymerase chain reaction. Cloning of the amplified fragments may be facilitated by incorporating restriction sites at the 5′ end of the primers. Preferably, a tissue-specific promoter used herein is specific for neuronal cells.

Inducible Promoters

The promoters of the present invention may also be promoters that respond to specific stimuli, for example promoters that bind steroid hormone receptors or HRE (hypoxic response elements) promoters that respond to hypoxic conditions, or low oxygen environments. See U.S. Pat. No. 6,265,390. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Enhancer

In addition, any of these promoters may be modified by the addition of further regulatory sequences, for example enhancer sequences. Chimeric promoters may also be used comprising sequence elements from two or more different promoters described above.

The term “enhancer” includes a DNA sequence which binds to other protein components of the transcription initiation complex and thus facilitates the initiation of transcription directed by its associated promoter.

The in vitro/in vivo/ex vivo expression of the RARβ2 of the present invention may be used in combination with a protein of interest (POI) or a nucleotide sequence of interest (NOI) encoding same.

POIs and NOIs

Suitable proteins of interest (POIs) or NOIs encoding same for use in the present invention include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives therof (such as with an associated reporter group). When included, the POIs or NOIs encoding same may be typically operatively linked to a suitable promoter, which may be a promoter driving expression of a ribozyme(s), or a different promoter or promoters, such as in one or more specific cell types.

Cytokines

In one aspect of the present invention the NOI(s) encodes a POI(s) wherein the POI is a cytokine or a cytokine receptor.

As used herein, the term “cytokines” refers to any varied group of proteins that are released from mammalian cells and act on other cells through specific receptors, said term also including said receptors. The term “cytokine” is often used interchangeably with the term “mediator”. Cytokines may elicit from the target cell a variety of responses depending on the cytokine and the target cell. By way of example, cytokines may be important in signalling between cells as inflammatory reactions develop. In the initial stages, cytokines such as IL-1 and IL-6 may be released from cells of the tissue where the inflammatory reaction is occurring. Once lymphocytes and mononuclear cells have started to enter the inflammatory site, they may become activated by antigen and release cytokines of their own such as IL-1, TNF, IL-4 and IFNγ which further enhance cellular migration by their actions on the local endothelium. Other cytokines, such as IL-8, are chemotactic or can activate incoming cells.

The term “cytokine” includes but is not limited to factors such as cardiotrophin, EGF, FGF-acidic, FGF-basic, flt3 Ligand, G-CSF, GM-CSF, IFN-γ, IGF-I, IGF-II, IL-1α, IL-1β, IL-2, IL-3, IL-4, IL-5, IL-6, IL-7, IL-8, IL-9, IL-10, IL-11, IL-12, IL-13, IL-15, IL-16, IL-17, IL-18 (IGIF), KGF, LIF, M-CSF, Oncostatin M, PDGF-A, PDGF-AB, PDGF-BB, SCF, SCGF, TGF-α, TGF-β₁, TNF-α, TNF-β, TPO and VEGF, as well as their cognate receptors.

Coupling

The RARβ2 of the present invention can be coupled to other molecules using standard methods. The amino and carboxyl termini of RARβ2 may be isotopically and nonisotopically labeled with many techniques, for example radiolabeling using conventional techniques (tyrosine residues-chloramine T, iodogen, lactoperoxidase; lysine residues—Bolton-Hunter reagent). These coupling techniques are well known to those skilled in the art. The coupling technique is chosen on the basis of the functional groups available on the amino acids including, but not limited to amino, sulphydral, carboxyl, amide, phenol, and imidazole. Various reagents used to effect these couplings include among others, glutaraldehyde, diazotized benzidine, carbodiimide, and p-benzoquinone.

Chemical Coupling

The RARβ2 of the present invention may be chemically coupled to isotopes, enzymes, carrier proteins, cytotoxic agents, fluorescent molecules, radioactive nucleotides and other compounds for a variety of applications including but not limited to imaging/prognosis, diagnosis and/or therapy.

Imaging

The use of labelled RARβ2 of the present invention with short lived isotopes enables visualization quantitation of RARβ2 binding sites in vivo using autoradiographic, or modern radiographic or other membrane binding techniques such as positron emission tomography in order to locate tumours with RARβ2 binding sites. This application provides important diagnostic and/or prognostic research tools.

Conjugates

In other embodiments, the RARβ2 of the invention is coupled to a scintigraphic radiolabel, a cytotoxic compound or radioisotope, an RARβ2 for converting a non-toxic prodrug into a cytotoxic drug, a compound for activating the immune system in order to target the resulting conjugate to a disease site such as a colon tumour, or a cell-stimulating compound. Such conjugates have a “binding portion”, which consists of the RARβ2 of the invention, and a “functional portion”, which consists of the radiolabel,

Individual

As used herein, the term “individual” refers to vertebrates, particularly members of the mammalian species, more in particular, humans.

Treatment

It is to be appreciated that all references herein to treatment include curative, palliative and prophylactic treatment.

Dosage

The dosage of the RARβ2 and/or pharmaceutical composition of the present invention will depend on the disease state or condition being treated and other clinical factors such as weight and condition of the individual and the route of administration of the compound.

Depending upon the half-life of the RARβ2 in the particular individual, the RARβ2 and/or pharmaceutical composition can be administered between several times per day to once a week. It is to be understood that the present invention has application for both human and veterinary use. The methods of the present invention contemplate single as well as multiple administrations, given either simultaneously or over an extended period of time.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

In addition or in the alternative the compositions (or component parts thereof) of the present invention may be administered by direct injection. In addition or in the alternative the compositions (or component parts thereof) of the present invention may be administered topically. In addition or in the alternative the compositions (or component parts thereof) of the present invention may be administered by inhalation. In addition or in the alternative the compositions (or component parts thereof) of the present invention may also be administered by one or more of: a mucosal route, for example, as a nasal spray or aerosol for inhalation or as an ingestable solution such as by an oral route, or by a parenteral route where delivery is by an injectable form, such as, for example, by a rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, intracavernosal, subcutaneous, transdermal or intramuscular) route.

By way of further example, the pharmaceutical composition of the present invention may be administered in accordance with a regimen of 1 to 10 times per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Disorders

The present invention is believed to have a wide therapeutic applicability.

For example, the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In particular, the present invention may be useful in the treatment of neurological disorders or nerve injuries such as spinal cord injury or avulsion injury as discussed herein.

Delivery

The delivery system for use in the present invention may be any suitable delivery system for delivering said NOI and providing said NOI is expressed in vivo to produce said associated peptide (e.g. RARβ2), which in turn provides the beneficial therapeutic effect.

The delivery system may be a viral delivery system. Viral delivery systems include but are not limited to adenovirus vector, an adeno-associated viral (AAV) vector, a herpes viral vector, retroviral vector, lentiviral vector, baculoviral vector. Alternatively, the delivery system may be a non-viral delivery system—such as by way of example DNA transfection methods of, for example, plasmids, chromosomes or artificial chromosomes. Here transfection includes a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Other examples of vectors include ex vivo delivery systems—which include but are not limted to DNA transfection methods such as electroporation, DNA biolistics, lipid-mediated transfection, compacted DNA-mediated transfection).

In a preferred aspect, the delivery system is a vector.

In a more preferred aspect, the delivery system is a viral delivery system—sometimes referred to as a viral vector such as a retroviral vector or lentiviral vector.

Vectors

As it is well known in the art, a vector is a tool that allows or faciliates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. Optionally, once within the target cell, the vector may then serve to maintain the heterologous DNA within the cell or may act as a unit of DNA replication. Examples of vectors used in recombinant DNA techniques include plasmids, chromosomes, artificial chromosomes or viruses.

The term “vector” includes expression vectors and/or transformation vectors.

The term “expression vector” means a construct capable of in vivo or in vitrolex vivo expression.

The term “transformation vector” means a construct capable of being transferred from one species to another.

Viral Vectors

In the present invention, the NOI may be introduced into suitable host cells using a viral delivery system (a viral vector). A variety of viral techniques are known in the art, such as for example infection with recombinant viral vectors such as DNA viruses, retroviruses, herpes simplex viruses, adenoviruses and adeno-associated viruses.

Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, gene delivery is typically mediated by viral infection of a target cell.

Herpes Virus Based Vectors

Herpes simplex viruses (HSV) I and II are large linear DNA viruses of approximately 150 kb encoding 70-80 genes. Like adenoviruses, HSV can infect a wide variety of cell types, including muscle, tumours, lung, liver and pancreatic islets. The viruses are able both to infect cells lytically and to establish latency in specific cell types, such as neurons. In order to use HSV as a vector, it is rendered replication defective. Following infection of a cell with HSV, the expression of a small number of immediate early (1E) genes is induced by a viral transactivating protein, VP16, which is carried into the cell as part of the viral tegument. The IE genes, which include ICPO, 4, 6, 22 and 27, are themselves regulators of gene expression that are important for the induction of the early and late genes required for viral replication and encapsidation. Mutation of ICP4 results in a virus unable to replicate except in a complementing cell line, but which still expresses the other IE gene products; these other E proteins are toxic to many cell types. Vectors defective for ICP4, 22 and 27 have been generated that have reduced levels of toxicity and prolonged gene expression in culture and in vivo. Herpes simplex virus can infect non-dividing cells of the mammalian nervous system.

An alternative approach to producing infectious HSV vectors is the use of amplicons. In this approach, a plasmid containing an HSV origin of replication and packaging sequence is cotransfected with cosmids containing the HSV genome but with a defective packaging sequence. The resulting virus particles contain only plasmid nucleic acid sequences, thereby eliminating any toxicity associated with low-level HSV-protein expression. This approach generates a helper free stock of virus.

HSV vectors have a large capacity for inserting heterologous DNA, allowing up to 50 kb to be included successfully, which may comprise multiple therapeutic genes. For example, four different antitumour genes have been inserted into a single HSV vector for use in cancer therapy. HSV vectors can be used to obtain highly regulated gene expression. An RU486-hormone-regulated chimeric transcription factor has been inserted into HSV along with a promoter containing binding sites for the regulated transcription factor; specific, regulated gene expression has been observed in vivo. Essentially all of the viral proteins may be deleted (gut-less vectors), still allowing around 106 viral particles to be produced per ml.

Adeno-Associated Viral Vectors

Adeno-associated virus (AAV) is a member of the parvovirus family, small single-stranded DNA viruses that require a helper virus, such as adenovirus or herpes-simplex virus, for replication. AAV is a human virus, with the majority of the population being seropositive for AAV, but no pathology has been associated with it. The virus contains two genes, rep and cap, encoding polypeptides important for replication and encapsidation, respectively. The wild-type virus can be grown to high titres and is able to integrate stably into a specific region of chromosome 19 following infection. The recombinant virus may not always integrate site-specifically. It has been suggested that this integration requires the presence of the rep protein. In wild-type virus infection, second-strand synthesis is stimulated by the presence of adenovirus E1 and E4 proteins; in the absence of adenovirus coinfection, cellular factors appear to dictate the rate of second-strand synthesis. In certain cell types, and/or following treatment with DNA-damaging agents, the rate of second-strand synthesis is high.

For the production of viral vectors, these two genes can be supplied in trans with only the inverted terminal repeats (ITRs) required in cis for viral replication. Therapeutic genes with the appropriate regulatory sequences can be inserted between the two ITRs, and the viral vector generated by cotransfection into the 293 cell line with a rep and cap expression vector and subsequent infection with a first-generation adeno-viral vector.

The degree of AAV infection of muscle, brain and liver cells with recombinant virus is exceedingly high in vivo. In these cell types, stable infection and gene expression apparently occurs independently of the helper virus. Injection of a β-galactosidase containing AAV vector into muscle also has resulted in β-galactosidase-positive myofibres for up to two years. Similarly, the injection of virus into the brain has resulted in long-term gene expression. AAV vectors containing human factor IX complementary DNA have been used to infect liver and muscle cells in immunocompetent mice. The mice produced therapeutic amounts of factor 1× protein in their blood for over six months, confirming the utility of AAV as a viral vector. AAV is highly suitable for the delivery of genes to specific target cells in vivo, preferably without inducing an immune response to the infected cells.

Retroviral Vectors

Examples of retroviruses include but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

A detailed list of retroviruses may be found in Coffin et al (“Retroviruses” 1997 Cold Spring Harbour Laboratory Press Eds: JM Coffin, SM Hughes, HE Varmus pp 758-763).

Preferred vectors for use in accordance with the present invention are recombinant viral vectors, in particular recombinant retroviral vectors (RRV) such as lentiviral vectors. Lentiviral vectors are able to deliver genes to non-dividing, terminally differentiated cells.

The term “recombinant retroviral vector” (RRV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RRV carries non-viral coding sequences which are to be delivered by the vector to the target cell. An RRV is incapable of independent replication to produce infectious retroviral particles within the final target cell. Usually the RRV lacks a functional gag-pol and/or env gene and/or other genes essential for replication.

Lentiviral genomes can be quite variable. For example there are many quasi-species of HIV-1 which are still functional. This is also the case for EIAV. These variants may be used to enhance particular parts of the transduction process. Examples of HIV-1 variants may be found in the HIV databases maintained by Los Alamos National Laboratory. Details of EIAV clones may be found at the NCBI database maintained by the National Institutes of Health.

EIAV vectors have been shown to deliver genes very efficiently to a number of neuronal cell types in vitro and in vivo. Gene expression has been sustained for a number of months in vivo, with little or no immunological reaction. Thus, according to the present invention EIAV vectors are a suitable delivery system to direct expression of RARβ2 in the human peripheral and central nervous systems and such systems are discussed in detail herein.

Vector titre may be estimated by infection assays. For example, infections could be carried out with vector preparation in question, and antibody staining for the product of the nucleotide of interest could be used to determine the proportion of productively infected cells, giving an indication of the titre of the vector preparation. For example, antibodies directed against RARβ2 are commercially available and may be advantageously utilised for this purpose according to the manufacturers' instructions. Alternatively, a PCR approach may be used, by amplifying using primers directed at the nucleotide of interest delivered by the vector, such as a nucleotide sequence directing the expression of RARβ2. Primers may advantageously be designed to include or comprise vector sequence(s) in order to ensure that the relevant amplification product has indeed originated from the sequence in question. Other ways in which vector titre may be estimated are known in the art, and are discussed in the Examples section hereinbelow.

Non-Viral Delivery

The pharmaceutically active agent (e.g. the RARβ2) may be administered using non-viral techniques.

By way of example, the pharmaceutically active agent may be delivered using peptide delivery. Peptide delivery uses domains or sequences from proteins capable of translocation through the plasma and/or nuclear membrane

Polypeptides of interest such as RARβ2 may be directly introduced to the cell by microinjection, or delivery using vesicles such as liposomes which are capable of fusing with the cell membrane. Viral fusogenic peptides may also be used to promote membrane fusion and delivery to the cytoplasm of the cell.

Preferably, the RARβ2 or fragment(s) thereof may be delivered into cells as protein fusions or conjugates with a protein capable of crossing the plasma membrane and/or the nuclear membrane. Preferably, the RARβ2 or fragment(s) thereof is fused or conjugated to a domain or sequence from such a protein responsible for the translocational activity. Preferred translocation domains and sequences include domains and sequences from the HIV-1-trans-activating protein (Tat), Drosophila Antennapedia homeodomain protein and the herpes simplex-1 virus VP22 protein.

Exogenously added HIV-1-trans-activating protein (Tat) can translocate through the plasma membrane and to reach the nucleus to transactivate the viral genome. Translocational activity has been identified in amino acids 37-72 (Fawell et al., 1994, Proc. Natl. Acad. Sci. U.S.A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. The third helix of the Drosophila Antennapedia homeodomain protein has also been shown to possess similar properties (reviewed in Prochiantz, A., 1999, Ann N Y Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chem, 269, 10444-50). This peptide has been used to direct biologically active substances to the cytoplasm and nucleus of cells in culture (Theodore, et al., 1995, J. Neurosci 15, 7158-7167). The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner. Any of the domains or sequences as set out above may be used to direct RARβ2 or fragment(s) thereof into cell(s). Any of the domains or sequences as set out above, or others identified as having translocational activity, may be used to direct the RARβ2 or fragment(s) thereof into a cell.

Pharmaceutical Compositions

The present invention also provides a pharmaceutical composition comprising administering a therapeutically effective amount of the agent of the present invention (such as RARβ2 and/or an agonist thereof as discussed herein) and a pharmaceutically acceptable carrier, diluent or excipients (including combinations thereof).

The pharmaceutical composition may comprise two components—wherein a first component comprises RARβ2 and a second component which comprises the agonist thereof. The first and second component may be delivered sequentially, simultaneously or together, and even by different administration routes.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Pharmaceutical Combinations

The agent of the present invention may be administered with one or more other pharmaceutically active substances. By way of example, the present invention covers the simultaneous, or sequential treatments with an agent according to the present invention and one or more steroids, analgesics, antivirals or other pharmaceutically active substance(s).

It will be understood that these regimes include the administration of the substances sequentially, simultaneously or together.

The invention will now be described by way of the following non-limiting Examples, given by way of illustration.

### EXAMPLES

Plasmid Construction

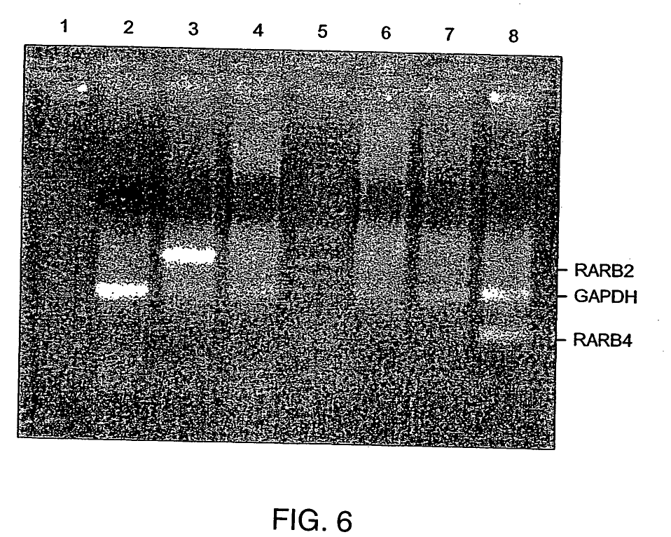

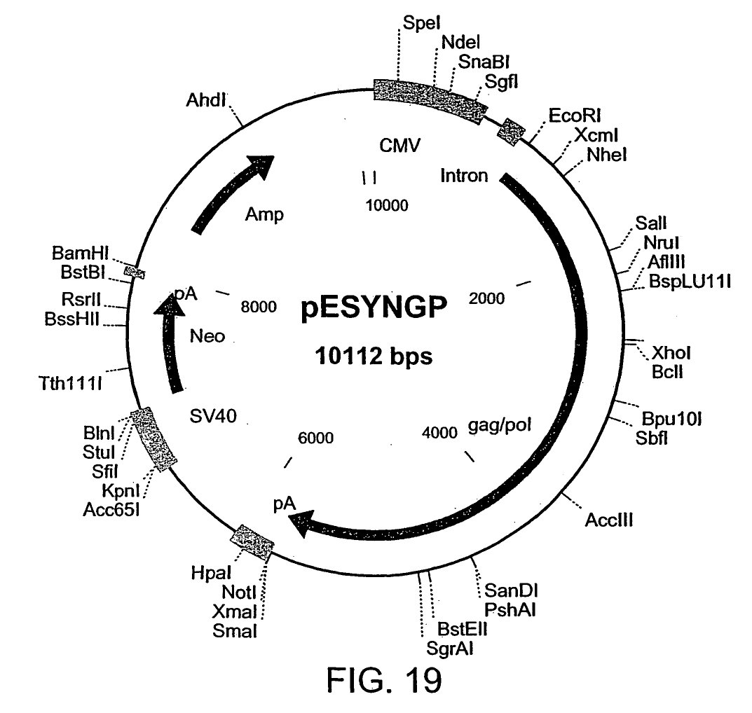

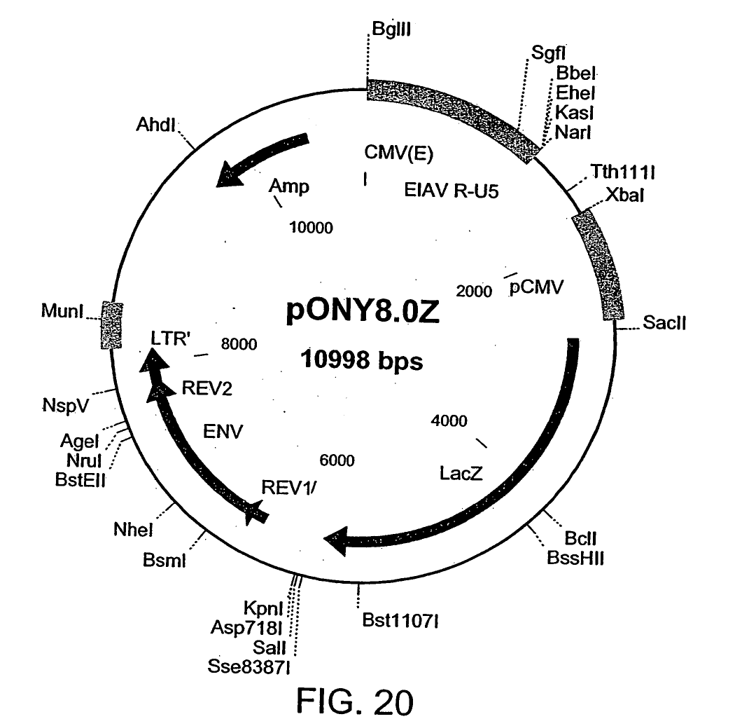

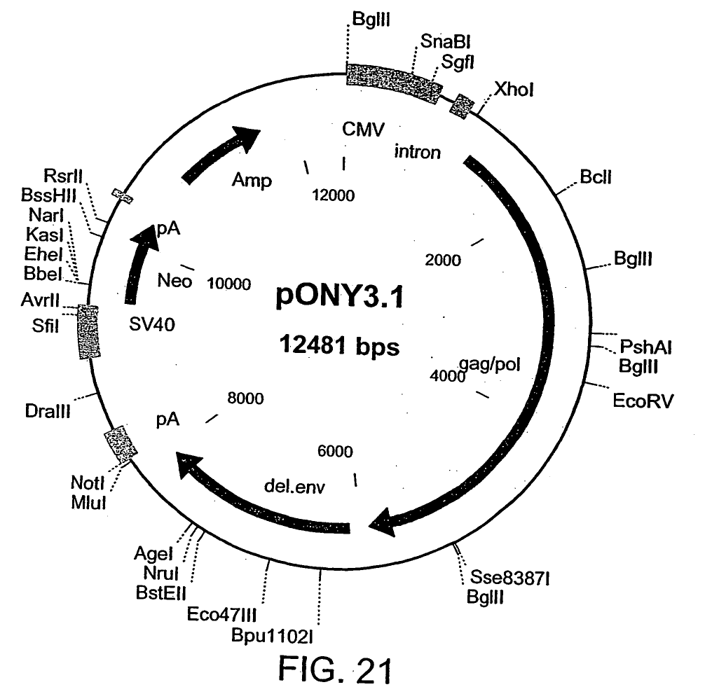

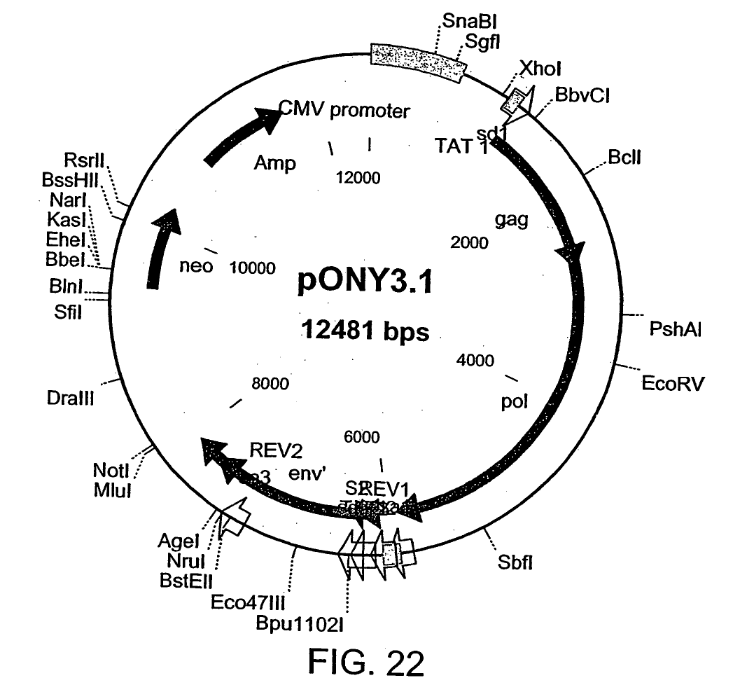

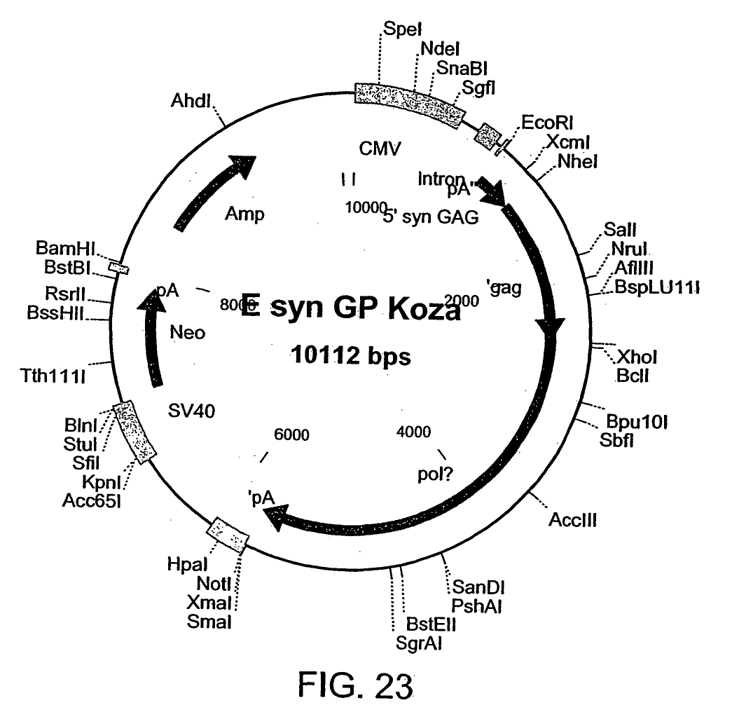

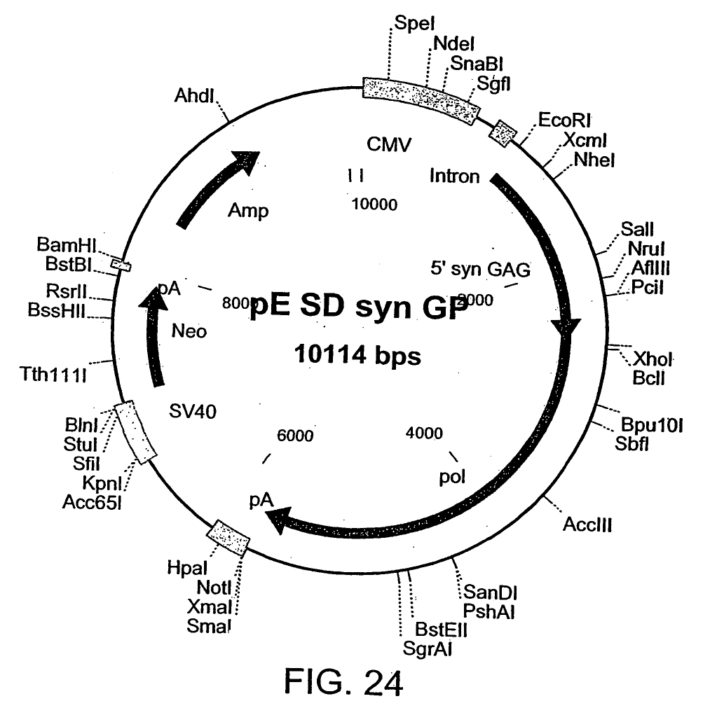

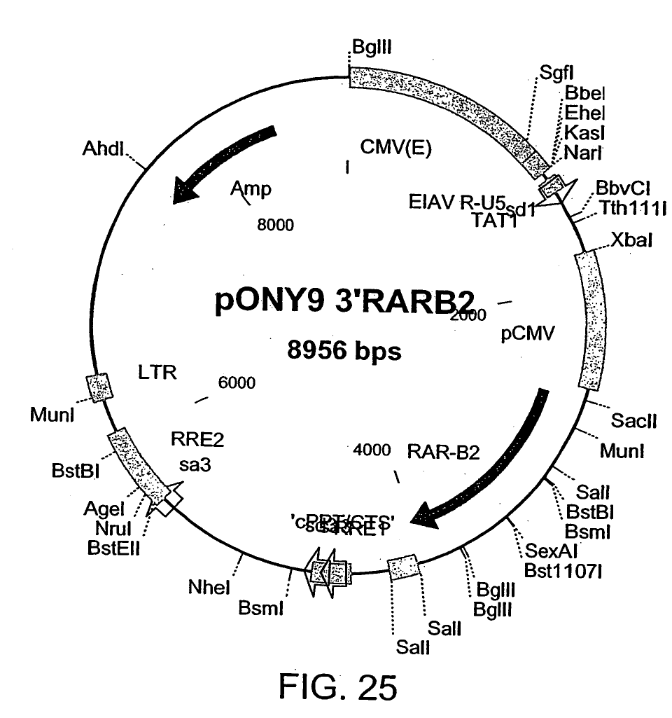

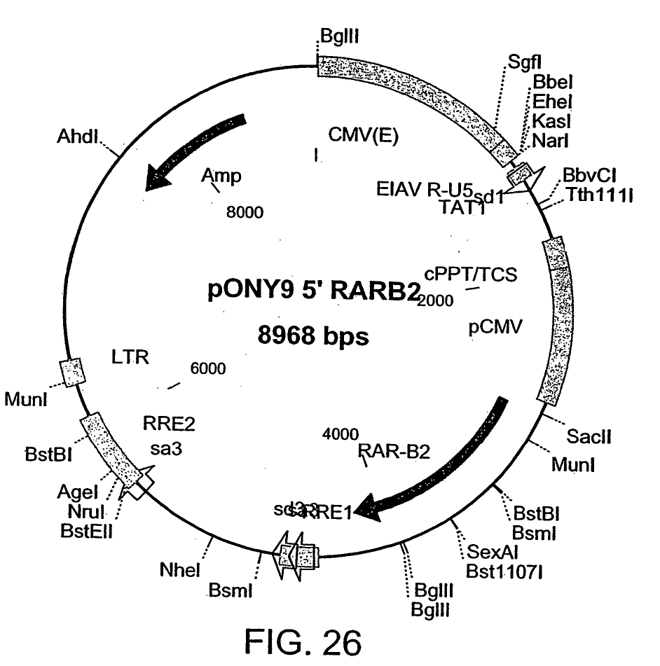

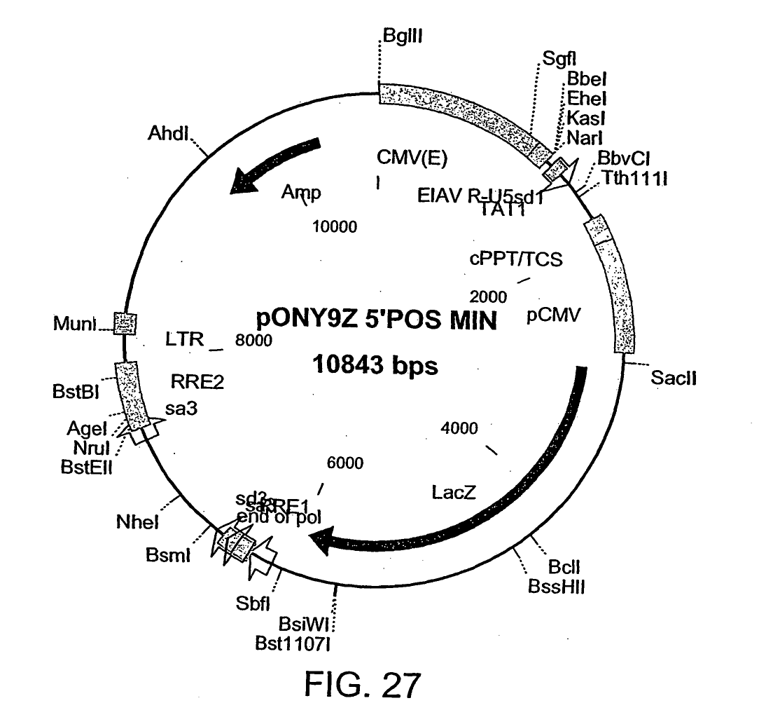

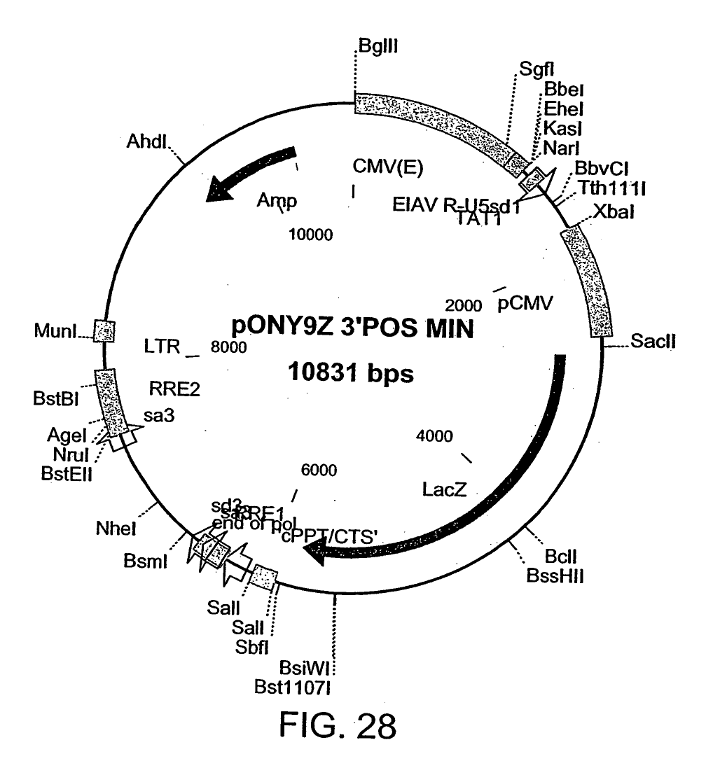

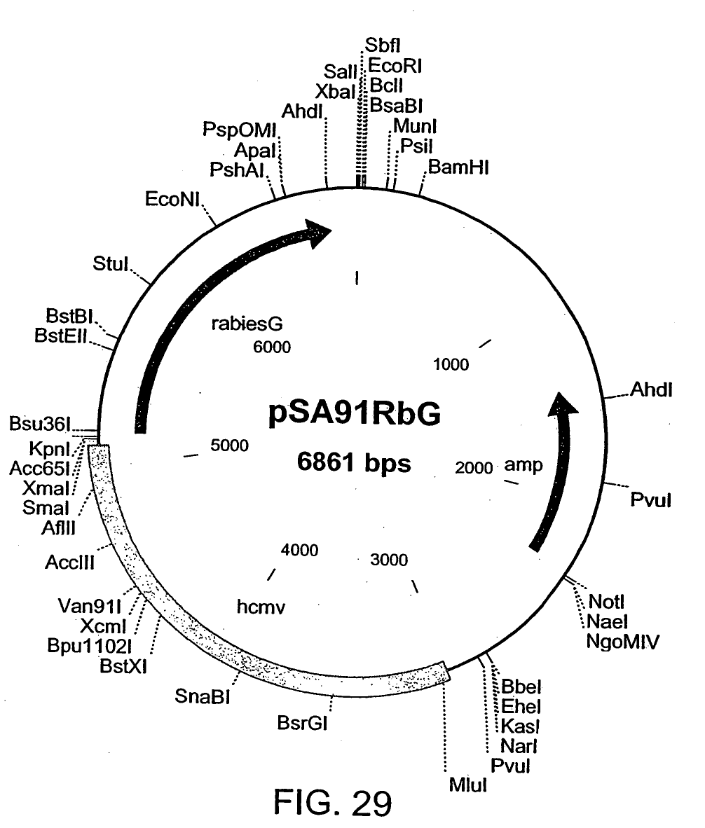

Numerous plasmids/constructs used in the following Examples (including pRV67, pRabG/pSA91RbG and others), are described in WO99/61639; pONY3.1 is described in WO 99/32646 (e.g. see Example 9, FIG. 6) and elsewhere; pSP72 is a standard Promega cloning vector, Genbank Acc. No. X65332; sources of other materials are as indicated or described herein, for example see FIGS. 19-21 and/or the accompanying sequence listing.

The plasmid used to express EIAV REV is pE syn REV which is a pCIneo based plasmid (Promega) which is made by introducing the EcORI to SalI fragment from a synthetic EIAV REV plasmid into the polylinker region of the pCIneo using the same sites. The synthetic EIAV REV plasmid made by Operon contains a codon-optimised EIAV REV open reading frame flanked by EcORI and SalI. The sequence of this fragment is shown in the sequence listing as codon optimised EIAV REV.

ESDSYNGP is made from pESYNGP by exchange of the 306 bp EcORI-NheI fragment, from just upstream of the start codon for gag/pol to approximately 300 base pairs inside the gag/pol ORF with a 308 bp EcORI-NheI fragment derived by digestion of a PCR made using pESYNGP as template and using the following primers: SD FOR [GGCTAGAGAATTCCAGGTAAGATGGGCGATCCCCTCACCTGG] and SD REV [TTGGGTACTCCTCGCTAGGTTC]. This manipulation replaces the Kozak concensus sequence upstream of the ATG in pESYNGP with the splice donor found in EIAV. The sequence between the EcORI site and the ATG of gag/pol is thus CAGGTAAG.

The codon-optimised EIAV gag/pol ORF is synthesised by Operon Technologies Inc., Alameda and supplied in a proprietary plasmid backbone, GeneOp. The complete fragment synthesised includes sequences flanking the EIAV gag/pol ORF: tctagaGAATTCGCCACCATG-EIAV gag/pol-UGAACCCGGGgcggccgc. The ATG start and UGA stop codons are shown in bold. XbaI and NotI sites are in lower case. These are used to transfer the gag/pol ORF from GeneOp into pCIneo (Promega) using the NheI and NotI sites in the latter.

pONY8.0Z construction: pONY8.0Z is derived from pONY4.0Z by introducing mutations which (1) prevent expression of TAT by an 83 nt deletion in the exon 2 of tat (2) prevent S2 ORF expression by a 51 nt deletion (3) prevent REV expression by deletion of a single base within exon 1 of rev and (4) prevent expression of the N-terminal portion of gag by insertion of T in ATG start codons, thereby changing the sequence to ATTG from ATG. With respect to the wild type EIAV sequence Acc. No. U01866 these correspond to deletion of nt 5234-5316 inclusive, nt 5346-5396 inclusive and nt 5538. The insertion of T residues is after nt 526 and 543.

### Example 1

### Stimulation of Neurite Outgrowth

Nerve growth factor acts via retinoic acid synthesis to stimulate neurite outgrowth in the peripheral nervous system.

Nerve growth factor (NGF) stimulates neurite outgrowth from cultured adult dorsal root ganglia (DRG)1. The vitamin A derivative retinoic acid (RA) also induces neurite outgrowth from various embryonic sources, including DRG 2,3. Are such similarities in effects of NGF and RA because they are both components of the same genetic cascade leading to neurite outgrowth? RA up-regulates low-and high-affinity NGF receptors 3,4 and induces the transcription of NGF itself 5, suggesting that RA may be upstream of NGF in the cascade. However, here we show the converse, namely, that NGF is upstream of RA. We show that when adult mouse DRG are cultured in the presence of NGF and a compound that inhibits enzymes involved in RA synthesis, neurite outgrowth does not occur. Conversely, when RA is added along with a blocking antibody to NGF, neurite outgrowth occurs as normal. We further show that NGF induces transcription of both the retinoic acid-synthesizing enzyme RALDH-2 and the retinoic acid receptor-1 as well as detectable release of synthesized RA. We propose that RA is required for adult DRG neurite regeneration and that NGF acts upstream of RA to induce its synthesis.

Cellular effects of RA are mediated by binding to nuclear receptors that are ligand activated transcription factors. There are two classes of receptors, retinoic acid receptors (RARs) and retinoid X receptors (RXRs), with three subtypes of each: α, β and γ. In addition, there are multiple isoforms of each subtype due to alternative splicing and differential promoter usage. RAR receptors mediate gene expression by forming heterodimers with the RXRs, whereas RXRs can mediate gene expression either as homodimers or by forming heterodimers with orphan receptors such as LXR8 An additional mechanistic association between NGF and RA pathways is suggested by the findings that the nuclear receptor NGFIB heterodimerizes with the RXRs8 and that NGFIB is rapidly induced in PC12 cells by the administration of NGF9.

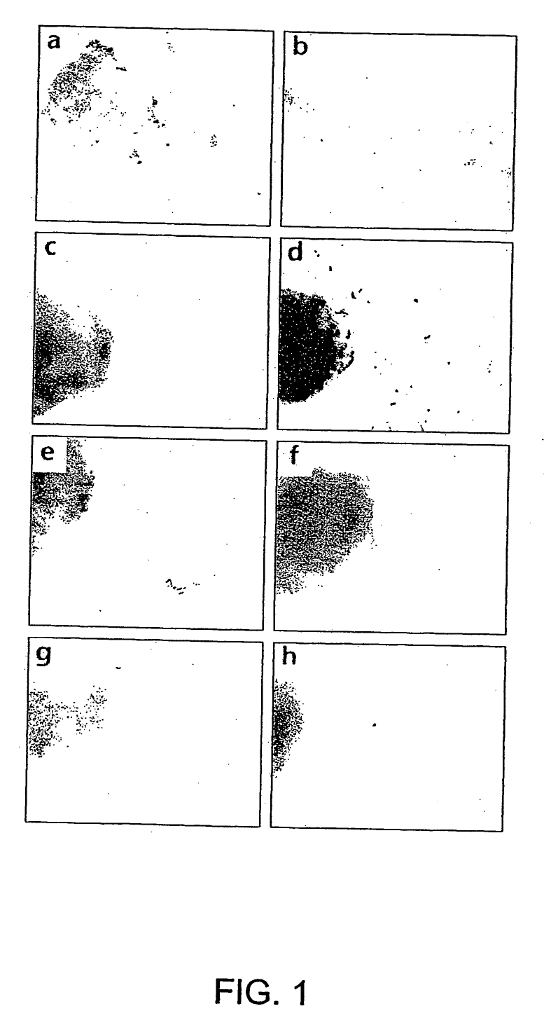

Although there is clearly a role for RA in the stimulation of neurite outgrowth from embryonic DRG2,3, it is not yet known if the same occurs in the adult DRG. To test this, we cultured adult mouse DRG in the presence of NGF (100 ng per ml), or RA (100 nM) in delipidated serum for five days. In both cases neurite outgrowth occurred (FIG. 1b). Little or no neurite outgrowth occurred in adult DRG cultured in only delipidated serum (FIG. 1a).

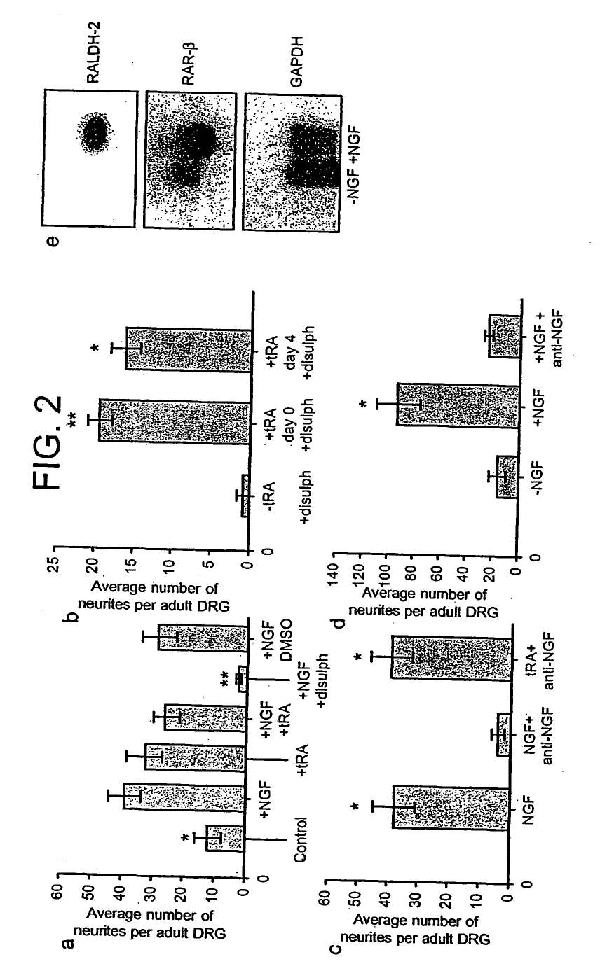

Differences in number of neurites were significant (FIGS. 2a; 1, 2 and 3). It is important to note that the number of neurites extended from RA- or NGF-treated adult DRG, although significantly greater than the number extended from untreated DRG, was smaller than the number obtained using embryonic tissue. When RA was added together with NGF, there was no additive effect of the two treatments (FIG. 1c), and no significant difference was seen between RA, NGF or RA plus NGF groups (FIGS. 2a; 2, 3 and 4). Although it may be that both NGF and RA are at individual saturating concentrations, the lack of synergy may also imply that NGF and RA act through the same pathway in order to cause neurite outgrowth. One could imagine either RA inducing the production of NGF5, or NGF inducing the production of RA by stimulating a RA-synthesizing enzyme.

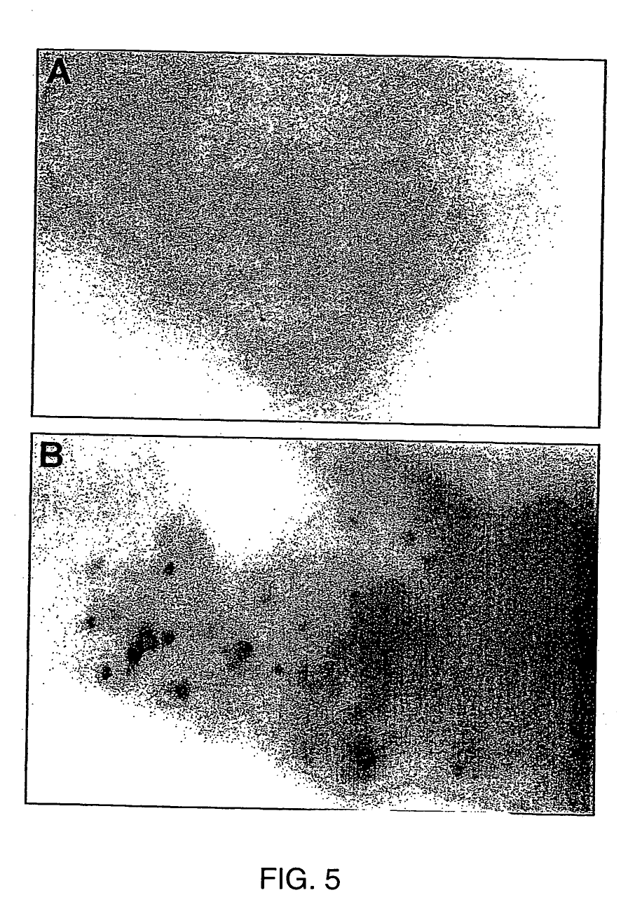

To test which of these hypotheses is most likely, we cultured adult DRG in the presence of NGF and 10 μM disulphiram, a compound which blocks the conversion of retinaldehyde to RA by inhibiting the enzyme aldehyde dehydrogenase10. If RA acts to stimulate NGF production then disulphiram should have no effect on NGF-stimulated neurite outgrowth, whereas if NGF induces RA synthesis then disulphiram should inhibit outgrowth. As shown in FIG. 1d, addition of 10 μM disulphiram along with NGF completely abolished NGF-induced neurite outgrowth (significant difference; FIGS. 2a, 2 and 5), whereas addition of DMSO (vehicle for disulphiram) and NGF did not affect neurite outgrowth (FIGS. 2a, 2 and 6). To confirm that disulphiram did not affect cell survival within the explants, we performed two types of rescue. In both cases, explants were cultured for eight days in medium supplemented with disulphiram. In the first rescue, 100 nM RA was added to the explants from the beginning of the experiment; in the second, RA was added on day 4. In both cases, significantly greater neurite outgrowth occurred compared to cultures grown in medium supplemented with disulphiram alone (FIGS. 1e, f and 2b). These experiments also confirm the specificity of disulphiram for the RA synthesis pathway, as RA can rescue the cellular response.

Inhibition of the inductive effect of NGF but not of RA by disulphiram suggests that NGF may precede RA in the cascade leading to neurite outgrowth. To test this, we used a blocking antibody against NGF In the presence of NGF and the blocking antibody, virtually no neurite outgrowth occurred (FIG. 1g; compare to DRG cultured in the presence of NGF alone, FIG. 1b). On the other hand, DRG cultured in the presence of the NGF-blocking antibody and 100 nM RA (FIG. 1h) showed neurite outgrowth equivalent to that obtained with NGF alone (FIGS. 1b and 2c).

If NGF is upstream of RA, it should induce synthesis of RA after addition to DRG cultures. To test this prediction, we used an F9 reporter cell line that responds specifically to the presence of RA due to transfection with 1.8 kb of the mouse RARβ2 gene promoter containing a retinoic acid response element (RARE) linked to the lacZ gene (Sonneveld, E., van den Brink, C. E., van der Leede, B. J., Maden, M. & van der Saag, P. T. (1999) Embryonal carcinoma cell line stably transfected with mRARb2-lacZ: sensitive system for measuring levels of active retinoids. Exp. Cell. Res. vol. 250 pp284-297). In the presence of RA, activated cells can be detected after β-galactosidase histochemical staining. We first eliminated the possibility that NGF itself activates F9 cells by growing them in the presence of NGF (100 ng per ml), whereupon there was no labeling of the F9 cells above background. We then cultured adult DRG in delipidated serum for five days under three different conditions: in the absence of NGF, in the presence of NGF or in the presence of both NGF and the NGF-blocking antibody. Cultured DRG were then sonicated and placed on the F9 reporter cells. NGF-treated DRG homogenates produced a clear RA signal relative to untreated DRG (FIG. 2d). This activation was prevented when the DRG were cultured with blocking antibody in addition to NGF (FIG. 2d).

We next considered which retinoic acid synthesizing enzymes might be induced by NGF. Retinol is converted by a two-step oxidative process to an aldehyde, retinal, which is then oxidized to retinoic acid (for review, see ref. 11). It has been shown that retinaldehyde dehydrogenase type 2 (RALDH-2) is expressed in the developing nervous system, including the DRG12. Using RT-PCR, we found strong induction of RALDH-2 by NGF in cultured adult DRG as well (FIG. 2e). Lastly, we also found up-regulation of the RARβ receptor in NGF-stimulated cultures (FIG. 2e), a phenomenon shown to be involved in neurite outgrowth 13.

Our results show that RA can stimulate neurite outgrowth from an adult neural tissue, the DRG. NGF similarly stimulates neurite outgrowth from this tissue, and we have demonstrated that it does so by inducing RA synthesis via an enzyme, RALDH-2. In the presence of either a NGF-blocking antibody or an inhibitor of RA synthesis, then NGF fails to act. Thus the most likely sequence of events in the induction of neurite outgrowth by NGF is: NGFRALDH-2RARAβ neurite outgrowth. We have not yet determined if NGF is directly responsible for inducing RALDH-2, or if some intermediary protein is required for this process. However, as NGFIB is one of the earliest genes induced by NGF9 and its product can heterodimerize with the RXRs8, the NGFIB/RXR heterodimer may be responsible for activating the RALDH-2 gene. Neurotrophins classically have been considered as potential agents for induction of nerve regeneration 14 and treatment of neurodegenerative diseases 15, but a major problem for their use is lack of effective modes of delivery to the site of the injury. Because RA is required for the regenerative response and it is downstream of NGF, then the problem of delivery to the lesion could be overcome, as RA is a low-molecular-weight lipophilic compound that can be administered orally. Thus, RA may be of clinical use in neurology.

### Example 2

### Induction of Neurite Development in Adult Neural Tissue

It is surprisingly shown herein that retinoic acid receptor-β2 induces neurite outgrowth in the adult mouse spinal cord.

Retinoic acid has been shown to be required for neurite outgrowth. We have recently demonstrated that the mechanism of it's action in peripheral nerve regeneration is by activating the retinoic acid receptor β-2. The adult central nervous system cannot regenerate. Therefore, we have investigated if regenerative failure in the adult spinal cord is related to the expression of retinoic acid receptor β2.

Results: We report here that in embryonic mouse spinal cord which can regenerate RARβ2 is up-regulated at concentrations which maximally stimulate neurite outgrowth. In contrast in the adult mouse spinal cord, RARβ2 is not detected nor is it induced by RA and no neurites are extended in vitro. When the adult cord is transfected with RAR β-2 neurite regeneration can be induced. There is no neurite outgrowth when the cord is transfected with another isoform of RARβ, RARβ4 This shows the importance of receptor specificity in neurite regeneration.

Conclusion: These data suggest that the loss in regenerative potential of the adult CNS is due in part to the loss of expression of RARβ2 and that it is intrinsic to the neuron itself. We suggest that gene therapy with RARβ2 may result in functional recovery of the injured spinal cord.

Background The induction of axonal regeneration in the adult central nervous system (CNS) is a major goal in neurobiology. The failure of CNS axons to regenerate under normal circumstances has been attributed to one or a combination of causes: the low abundance of neurotrophic factors; the absence of growth-promoting molecules; the presence of growth-inhibiting molecules. Thus attempts to restimulate axon growth in the CNS have centered on these three possible. When peripheral nerve grafts were used to provide a permissive environment then spinal cord and medulla neurons extended axons up to 30 mm in the adult rat¹. A similar strategy combined with fibroblast growth factor application resulted in the partial restoration of hind limb function². Neutralisation of neurite growth inhibitors present in myelin with antibodies permitted longer extension of axons than in control young rats³ and led to the recovery of specific reflex and locomotor functions after spinal cord injury⁴. A combination of neurotrophin-3 and these antibodies was successful in inducing long distance regeneration of corticospinal tract (CST) axons⁵. A suspension of olfactory ensheathing cells was also effective in returning locomotor function to the lesioned CST of rats⁶. If neurotrophins act simply to keep axotomised neurons alive⁷ then in these methodologies for inducing regeneration it is the environment surrounding the axons which is the focus of attention rather than the intrinsic capabilities of the neuron itself. However, at least part of the regenerative loss of the CNS is intrinsic to the neuron itself (4 refs). This suggests that the identification of genes that are not expressed in the non regenerating adult CNS but are in the developiong CNS which can regenerate neurites may lead to new stratagies of treatment of spinal cord injuries by gene therapy.

We show here that one such gene is RARβ2 which is activated by retinoic acid (RA) the biologically active metabolite of vitamin A. RA is present in various tissues of the developing embryo and adult animal, especially the nervous system⁸⁻¹³. In its absence, developing neurons of the CNS do not extend neurites into the periphery^{14,15}. Conversely, when applied to cultured neurons, RA induces both a greater number and longer neurites¹⁶ as well as being capable of dictating their direction of growth¹⁷. RA acts at the level of gene transcription because it is a ligand for two classes of nuclear transcription factors, the retinoic acid receptors (RARs) and the retinoid X receptors (RXRs)^{18,19}. There are three members of each class of retinoid receptor a, b and g as well as several isoforms of each member and this diversity may be responsible for the pleitropic effects of RA on cells.

We have been studying the molecular mechanisms of action of RA on neurons and have concluded that one of these retinoic acid receptors, RARβ2 is the crucial transducer of the RA signal in neurons as it is up-regulated in situations where RA stimulates neurite outgrowth²⁰. We hypothesised therefore that the absence or below threshold level of this nuclear receptor in the adult spinal cord may contribute to the failure of this tissue to regenerate axonal projections.

Effect of RA on Embryonic Mouse Spinal Cord In Vitro.

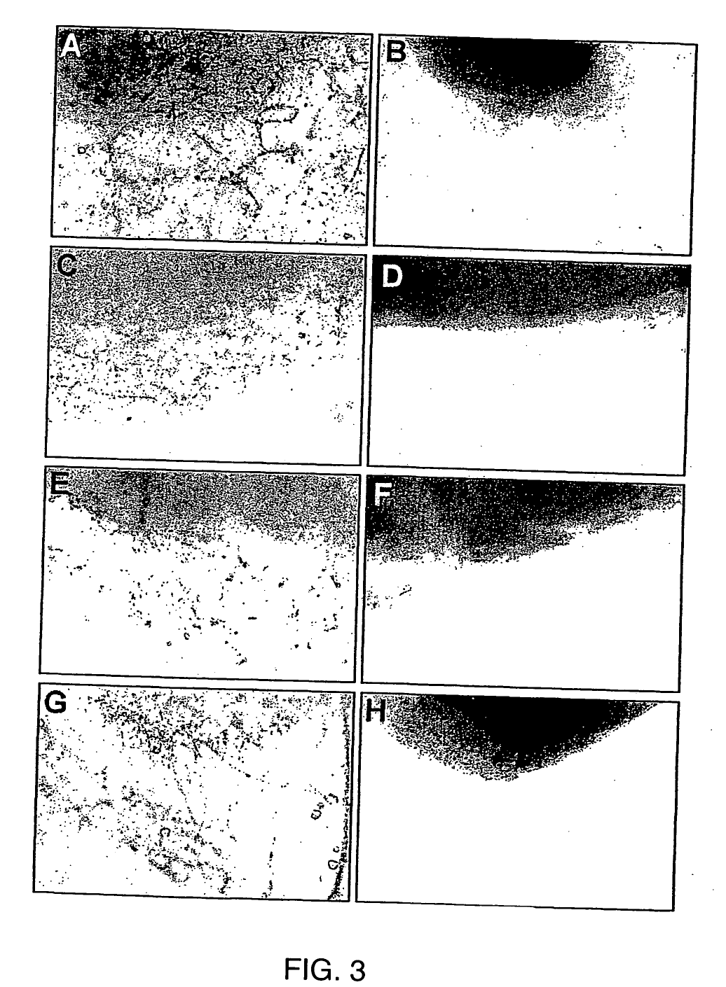

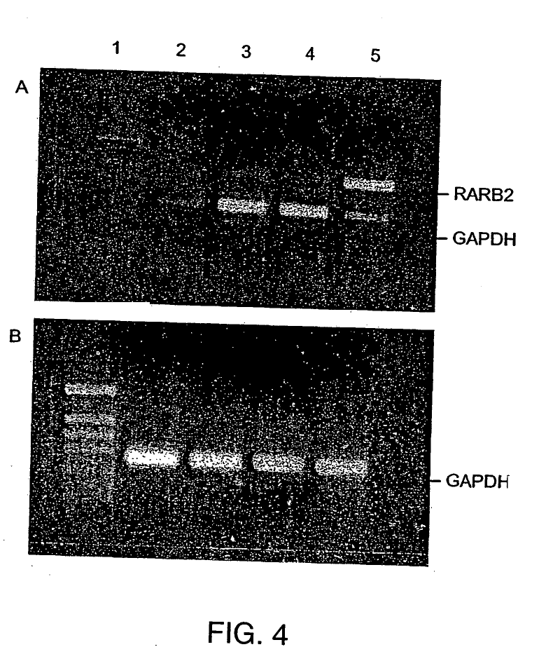

We began by confirming that the mouse embryonic spinal cord will respond to RA by extending neurites as do other areas of the embryonic CNS^{12,17, 21-23} and that this behaviour involves an up-regulation of RARβ2. E13.5 spinal cord was dissected from mouse embryos placed in a cellogen matrix and cultured in 10% delipidated serum. All-trans-RA was added at 3 different concentrations (10⁻⁸M, 10⁻⁷M, 10⁻⁶M) and after 5 days the explants were stained with a neurofilament antibody and examined for the presence of neurites. There was an increasing number of neurites emerging from the cultured cord with increasing concentrations of RA with the maximal effect at 10⁻⁶M (FIG. 3C, E, G). Even in the absence of RA the embryonic cord extended neurites (FIG. 3A) presumably because of the high endogenous content of RA and its precursor retinol^{9,13}. Indeed, when the endogenous synthesis of RA is inhibited with disulphiram then no neurites are extended²⁴. To demonstrate that the induction of neurites involved the up-regulation of RARβ2, RT-PCR was performed on cultures after 5 days in the same range of RA treatments. This revealed that RARβ2 is normally expressed in embryonic spinal cord at all concentrations of RA used (FIG. 4A, lanes 1-5) and that it is strongly up-regulated after 1×10⁻⁶ M RA treatment (FIG. 4A, lane 5), the same concentration which gives maximal neurite outgrowth.

Lack of Effect of RA on Adult Mouse Spinal Cord In Vitro.

We next performed an identical series of experiments using 10 month old adult spinal cord rather than the embryonic cord. In contrast to the embryonic cord, RA had no effect on neurite outgrowth at any concentration tested and like the untreated controls, these RA treated adult cords failed to extend any neurites at all (FIG. 3B, D, F, H). Examining the involvement of RARβ2 by RT-PCR revealed that control adult spinal cord had little or no detectable endogenous levels of this receptor (FIG. 4B, lane 1) and that there was no change in its level in response to RA treatment at any concentration (FIG. 4, lanes 2-5), unlike the embryonic cord.

Induction of Neurites in Adult Spinal Cord

We therefore hypothesised that it was the lack of RARβ2 expression which may be responsible for the completely inert behaviour of the adult spinal cord. Our previous observations that adult DRG which do respond to RA by extending neurites also up-regulate RARβ2²⁴ demonstrates that the same behaviour is elicited by embryonic and the appropriate adult neurons and reinforces the differences in regulative behaviour between PNS and CNS neurons. To test our hypothesis we used a defective herpes simplex virus type 1 (HSV-1) vector to transfect pieces of adult (10 months) mouse spinal cord.

Three different transfections were performed, two of which served as controls. Firstly, just the vector containing lacZ (pHSVlacZ); secondly the vector containing RARβ2 (pHSVRARβ2); thirdly the vector containing another isoform of the RARβ gene, RARβ4 (pHSVRARβ4). The latter served as a very precise control for transfection since we do not detect the RARβ4 isoform after RA treatment of neurons in our previous experiments²⁰ hence it is not involved in neurite outgrowth. We first ensured that the transfections were successful and that the relevant receptor isoform was expressed in the cultured cord. Pieces of spinal cord were transfected overnight with the appropriate construct and analysed either three or four days later. The pHSVlacZ treated cords showed a significant amount of transfection had taken place as judged by b-galactosidase staining of the adult cord (FIG. 5B). RT-PCR demonstrated that transfection with the RARβ2 vector resulted in the expression of RARβ2 (FIG. 6, lane 3) but not RARβ4 (FIG. 6, lane 4) and transfection with the RARβ4 vector resulted in the expression of RARβ4 (FIG. 6, lane 8) but not RARfl2 (FIG. 6, lane 7). In the non transfected cord neither RARβ2 or RARβ4 were detected (FIG. 6, lanes 2 and 6).

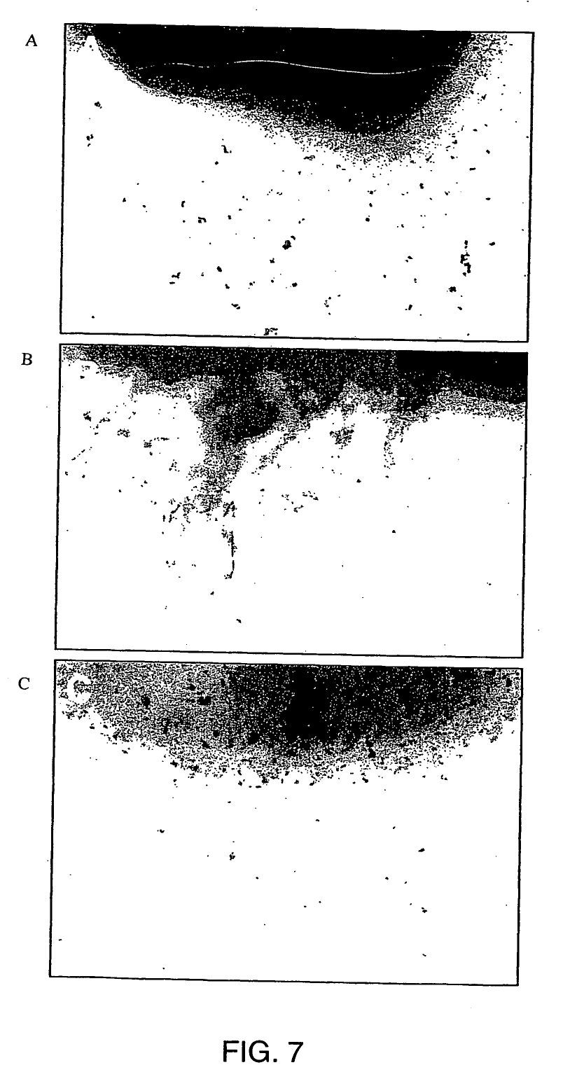

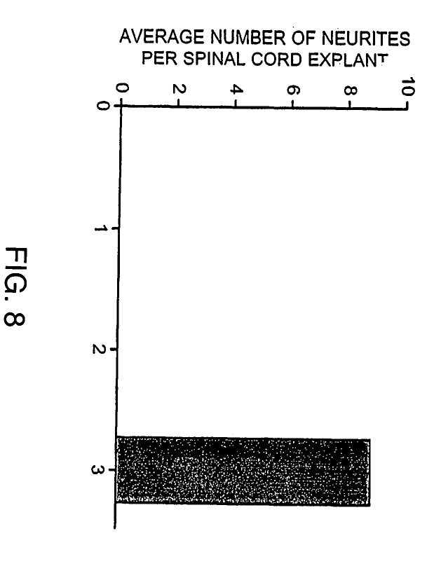

The effects of these transfections on neurite outgrowth were clear-cut. Transfection with the pHSVlacZ failed to change the behaviour of the cultured adult cord which remained completely un-responsive in terms of neurite outgrowth (FIG. 7A, 12/12 transfections). Similarly, the transfections with pHSVRARβ4 produced no response in the cultured cord which remained inert (FIG. 7C, 12/12 transfections). However, transfections with the pHSVRARβ2 isoform clearly produced a different behaviour and many neurites appeared in the cultures (FIG. 7B, 8/12 transfections). The number of neurites produced in the pHSVRARβ2 cord varivaried between 3 and 23. In the pHSVlacZ transfections there was considerable variability in the number of lacZ-positive cells per explant. This suggests that the variability in neurite number may be due to variability in number of cells transfected.

These results provide strong support for our hypothesis that the RARβ2 isoform plays a key role in the induction of neurite outgrowth in response to RA and that this may be a crucial component which fails to be up-regulated in the injured adult CNS. Our hypothesis is based upon several experiments involving either regenerating or non-regenerating neuronal tissues and their response to RA. Thus the embryonic mouse spinal cord, the embryonic mouse DRG and the adult mouse DRG all respond to RA by up-regulating RARβ2 and extending neurites. In contrast, the adult mouse spinal cord fails to up-regulate RARβ2 and fails to extend neurites. Furthermore, NGF stimulates neurite outgrowth and acts by up-regulating RARβ2 ²⁴ and neurite outgrowth from embryonic mouse DRG can be stimulated by a RARβ agonist²⁰.

These results reveal that when the genome of the neuron itself is manipulated then regeneration can be reawakened. This is in contrast to the recent inductions of neurite outgrowth in vivo which have concentrated on the inhibitory factors present in the CNS environment¹⁻⁵. During development the loss of regenerative capacity of the spinal cord correlates with the appearance of myelin associated neurite growth inhibitory molecules and some of these are thought to be produced by the oligodendrocytes²⁵. Either the regeneration of neurites we see in our cultures and that seen in cultures where the environment has been manipulated are two different mechanism of neurite regeneration or they are related processes. Support for the latter view is provided by the fact that CNS neurons themselves have been shown to be involved in myelination²⁶. Therefore it is tempting to speculate that the presence of RARβ2 in neurons during development may regulate genes involved in myelination, and that this process is recapitulated by transfection of the RARβ2 gene in the adult CNS.

None of the neurites we observed in the RARβ2 transfected cord elongated over an appreciable distance. This suggests that elongation of the neurite may require the expression of a different set of genes. Evidence that this may be true is shown from regeneration of axons in the adult PNS, where a transition from arborizing to elongating growth depends upon a transcriptional dependent switch²⁷. Alternatively the failure of elongation of neurites in our cultures may be due to the fact that there is likely to be a loss of expression of RARβ2 over time due to the transient nature of the transfections and that this does not allow enough time for elongation to occur.

Nonetheless we propose that these preliminary data support a role of RARβ2 in the regeneration of neurites in the adult CNS and that gene therapy with this transcript in combination with other treatments may one day lead to functional recovery of the injured spinal cord.

Methods

Cultures. Spinal cord was dissected from either E13.5 or 10 month old mice and cut into transverse pieces of about 5 mm. These were cultured in cellogen matrix (ICN flow), prepared by mixing 1 volume of 7.5% sodium bicarbonate, 1 volume of 10×MEM (Gibco) and 8 parts cellogen (ICN flow). The pH was adjusted to 7.5 by dropwise addition of 5M NaOH. Explants were fed every two days. The media consisted of DMEM-F12 with glutamine (Gibco), 6% glucose, GMS-A (Gibco) 10% delipidated serum and all-trans-RA (stock solution, 1×10⁻⁵ M, Sigma). On the fifth day they were fixed in 4% paraformaldyde and stained with the neurofilament antibody, NF200 (Sigma).

RT-PCR analysis. RNA was extracted (trizol, Gibco) and cDNA prepared by the use of a Pharmacia kit as described in the manufactures instructions. The primers used were from GAPDH, RARβ2 and RARβ4. PCR was carried out for 25 cycles for embryonic spinal cord and 40 cycles for adult spinal cord. Amplification was carried out as follows, denaturation for 30 s at 95° C., annealing for 30 s at 55° C. and extension for 1 min at 72° C. One fifth of the resultant product was then run on a gel.

Transfections. Virus stocks were prepared and β galactosidase staining carried out as described in ref²⁸. The titres used were: pHSV RARβ2, 5×10⁻⁴ ip/ul, pHSVRARβ4, 4×10⁻⁴ ip/ul, pHSVlacz 5×10⁻⁴ ip/ul.

### Example 3

### Neurite Outgrowth from Mouse Dorsal Root Ganglion Neurons

This Example demonstrates stimulation of neurite outgrowth by retinoic acid/RARβ in the peripheral nervous system. The role of retinoic acid receptors in neurite outgrowth from different populations of embryonic mouse dorsal root ganglia.

Dorsal root ganglion (DRG) neurons can be categorised into at least three types based upon their neurotrophin requirement for survival. We have analysed the expression of the retinoic acid receptors (RARs) and the retinoid X receptors (RXRs) in NGF, NT-3 and BDNF dependent neurons isolated from embryonic day 13.5 mouse DRG. We show that each population of neurons expressed each of the three RXRs, a p and y. However, whilst the NGF and NT-3 dependent neurons expressed each of the RARs α, β and γ the BDNF dependent neurons only expressed RARα and β. When retinoic acid was added to each of the neuronal classes only the NGF and NT-3 dependent neurons responded by extending neurites, and this response involved the up-regulation of RARβ₂. This specificity was confirmed by the use of receptor selective agonists as only a RARβ selective compound stimulated neurite outgrowth. These results suggest a role for RA acting via RARβ₂ in the outgrowth of neurites.

The neurotrophins are a family of growth factors that are required for the survival of a variety of primary sensory neurons in the developing peripheral nervous system (Snider, 1994). The family includes nerve growth factor (NGF) (Levi-Montalcini, 1987) neurotrophin-3 (NT-3) (Maisonpierre et al., 1990) and brain-derived neurotrophic factor (BDNF) (Barde et al., 1982). They are synthesised in the target fields innervated by peripheral neurons and are thought to be transported by a retrograde mechanism from the target field to support the survival of the developing neurons. The neurotrophins act through receptor tyrosine kinases designated Trk. NGF specifically activates TrkA; BDNF activates TrkB and NT-3 activates TrkC (reviewed in Snider, 1994). Analysis of the phenotypes resulting from loss of function experiments of the neurotrophins and the receptor tyrosine kinases have revealed that the dorsal root ganglia (DRG) neurons can be classified into at least three types. Neurons that require NGF for their survival mediate nocioceptive (pain) and thermal receptive functions. In the periphery the axons terminate in the superficial layers of the skin and innervate the superficial laminae of the spinal cord (Crowley et al., 1994; Smeyne et al., 1994). Proprioceptive neurons (sense of position of the limbs in space), which are much larger than NGF type neurons, project into the periphery to the primary endings of muscle spindles and extend a collateral branch to the motor pools in the spinal cord are dependent upon NT-3 for their survival (Emfors et al., 1994; Farinas et al., 1994; Klein et al., 1994). BDNF neurons are small to medium sized and may include some classes of the mechanoreceptors (Klein et al., 1993; Jones et al., 1994).

In addition to growth factors being involved in the survival of neurons, retinoids can also carry out the same role. Retinoids are a family of molecules derived from vitamin A and include the biologically active metabolite, retinoic acid (RA). The cellular effects of RA are mediated through the action of two classes of receptors, the retinoic acid receptors (RARs) which are activated both by all-trans-RA (tRA) and 9-cis-RA (9-cis-RA), and the retinoid X receptors (RXRs), which are activated only by 9-cis-RA (Kastner et al., 1994; Kleiwer et al., 1994). The receptors are of three major subtypes, α, β and γ, of which there are multiple isoforms due to alternative splicing and differential promoter usage (Leid et al. 1992). The RARs mediate gene expression by forming heterodimers with the RXRs, whilst the RXRs can mediate gene expression as homodimers or by forming heterodimers with a variety of orphan receptors (Mangelsdorf & Evans, 1995). Interestingly, one of the earliest genes induced by NGF in PC12 cells is the orphan receptor NGFI-B (NURR1) (Millbrandt, 1989). This suggests that the growth factor and retinoid mediated pathway in developing neurons can interact. This interaction may be critical for the survival of the neuron because RA has been shown to be involved in the survival and differentiation of neurons (Wuarin and Sidell, 1991; Quinn and De Boni, 1991). Furthermore, many studies on a variety of embryonic neuronal types have shown that RA can stimulate both neurite number and length (reviewed in Maden, 1998) as indeed, can the neurotrophins (Campenot, 1977; Lindsay, 1988; Tuttle and Mathew, 1995). Recently we have shown that RA is critical for neurite regeneration in adult DRG and that it's synthesis may be regulated by NGF (Corcoran and Maden, 1999).

In the work described here, we use E13.5 mouse DRG to investigate further the nature of the interaction between the retinoid mediated pathway and the growth factor pathway by asking which of the RARs and RXRs are expressed in neurons that are dependent upon different neurotrophins for their survival. We show that a different retinoid receptor profile is indeed expressed in NGF, NT-3 and BDNF neurons and that this profile is altered after an RA treatment which induces neurite outgrowth. Specifically, RARβ2 is up-regulated in NGF and NT-3 neurons but not in BDNF type neurons. This result was confirmed by the use of receptor selective agonists, as only the RARβ agonist will substitute for RA in inducing neurite outgrowth. These results suggest a role for RA acting via RARβ2 in the outgrowth of neurites.

Materials and Methods

DRG cultures. DRG were obtained from E13.5 mice, freed of non-ganglionic tissue and collected in ice-cold calcium magnesium free phosphate buffered saline. To prepare dissociated cell suspensions the ganglia were treated with 0.05% trypsin for 15 minutes at 15° C. The reaction was stopped by the addition of 1% serum and single cells obtained by trituration with a 23 G needle. The cells were then spun at 1000 g for ten minutes and resuspended in media. They were plated out at a density of approximately of 25000 cells/cm² in wells that had been precoated with 100□g/ml poly D lysine for 2 hrs. The cultures were fed every 2 days.

Culture media consisted of DMEM-F12 with glutamine (Gibco), 6% glucose, ITS (Gibco). The growth factors used were either 50 ng/ml NGF (7s, Promega) 50 ng/ml NT3 (Promega) or 50 ng/ml BDNF (Promega). Retinoids were used at a concentration of 1×10⁻⁷ M. All-trans-retinoic acid was obtained from Sigma and the receptor agonists were synthesised by CIRD Galderma: CD366 activates RARA CD2019 activates RARβ CD437 activates RARy and CD2809 activates all of the RXRs.

RT-PCR analysis. RNA was extracted (trizol, Gibco) and cDNA prepared by the use of a Pharmacia kit as described in the manufacturer's instructions. The primers used were from mouse RARs, RXRs and GAPDH. In order to identify which RAR/RXR receptors were involved neurite outgrowth semi quantitative PCR was used. Amplification was carried out in the linear range for each RAR and RXR and their levels of expression were compared to GAPDH. For the RXRs 28 cycles were performed, while 25 cycles were used for RARα and RARγ and 22 cycles for RARβ and GAPDH. Amplification was carried out as follows, denaturation for 30 s at 95° C., annealing for 30 s at 55° C. and extension for 1 min at 72° C. One fifth of the resultant product was then run on a gel and blotted. This was then probed with the appropriate RAR, RXR or GAPDH for nommalisation.

In situ Hybridisation: Cells were washed once with PBS and fixed in 4% PFA for 30 mins. They were then washed twice for 5 mins in PBS-0.05% Tween (PBT). Hybridisation was carried out at 55° C. overnight. The buffer consisted of 0.1M Tris-Ci, pH9.5, 0.05M MgCl2, 0.1M NaCl and 0.1% Tween-20. The cells were then washed sequentially for 15 min. at 65° C. in 50% hybridisation buffer, 50% 2×SSC, 100% 2×SSC, and finally in 0.2% SSC. They were then washed at RT for 5 minutes each in 75% 0.2×SSC, 25% PBT, 50% 0.2×SSC, 50% PBT, 25% 0.2×SSC, 75% PBT, and 100% PBT. The cells were blocked in 2% sheep serum in PBT for 1 hr and incubated with anti DIG antibody overnight at 4° C. The cells were then washed 8 times in PBT for 2 hrs. Colour was developed by using NBT/BCIP according to the manufacturer's instructions (Boehringer Mannheim).

Immunohistochemistry and measurement of neurite length: Cells were washed once with PBS and fixed in 4% PFA for 30 mins. They were then washed twice for five minutes in PBS-0.05% Tween (PBT). They were then incubated in primary antibody NF200 (sigma) at 4° C. overnight and washed 8 times for 2 hrs in PBT. Secondary antibody was then applied for 2 hrs at RT, and the cells again washed 8 times for 2 hrs in PBT. They were then incubated for 5 mins. in PBS containing 0.5 mg/ml DAB and 6% H₂O₂. Neurite length was measured by using NIH image software. The experiments were repeated three times and three random fields were taken for each experiment for analysis. On average there were 40 neurons in each field and the longest neurite branch was measured for a given neuron.

Results

Expression of Receptors by In Situ Hybridisation.

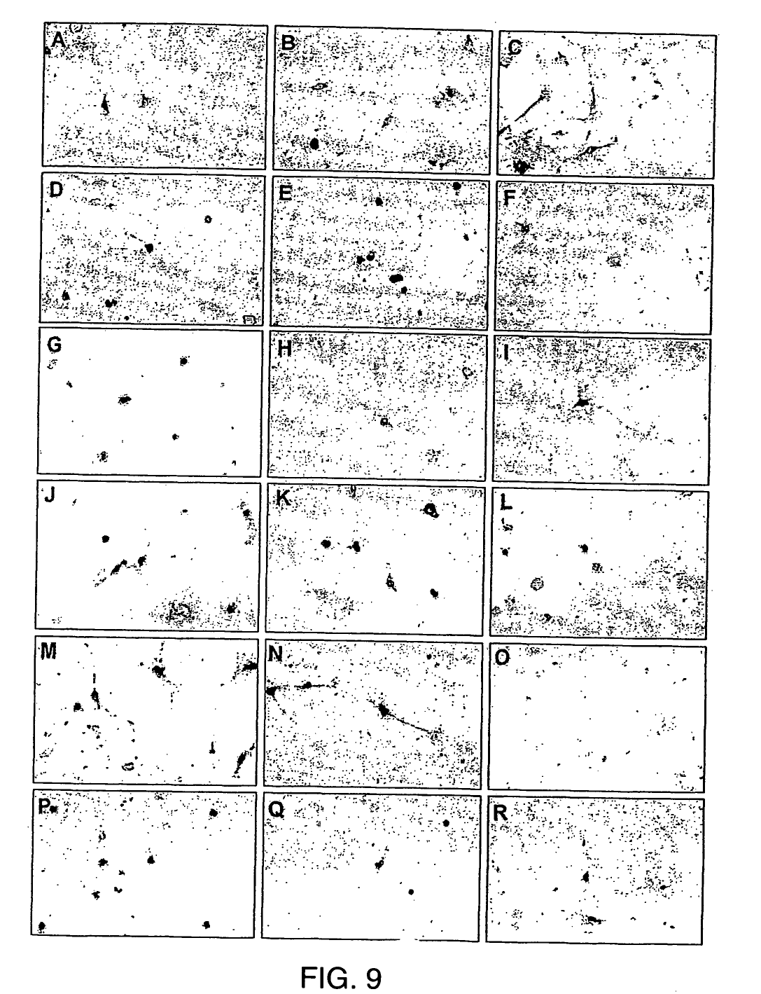

We first examined the expression of the RARs and the RXRs in primary cultures of E13.5 mouse DRG by in situ hybridisation. Dissociated DRG neurons were cultured in serum free medium either in the presence of NGF, NT-3 or BDNF for a period of five days. In the absence of neurotrophins the cells died. We found that all three types of neurons expressed RXRα (FIG. 9D, 9J, 9P), RXRβ (FIG. 9E, 9K, 9Q) and RXRγ (FIG. 9F, 9L, 9R). In contrast, the RARs showed a differential expression between the three types of neurons. Whilst the NGF and NT-3 dependent neurons expressed RARα (FIG. 9A, 9G), RARβ (FIG. 9B, 9H) and RARγ (FIG. 9C, 9I), the BDNF dependent neurons only expressed RARα (FIG. 9M) and RARβ (FIG. 9N). RARγ was not detectable by in situ hybridisation in the BDNF dependent cultures (FIG. 9O).

Effect of RA on Neurite Outgrowth

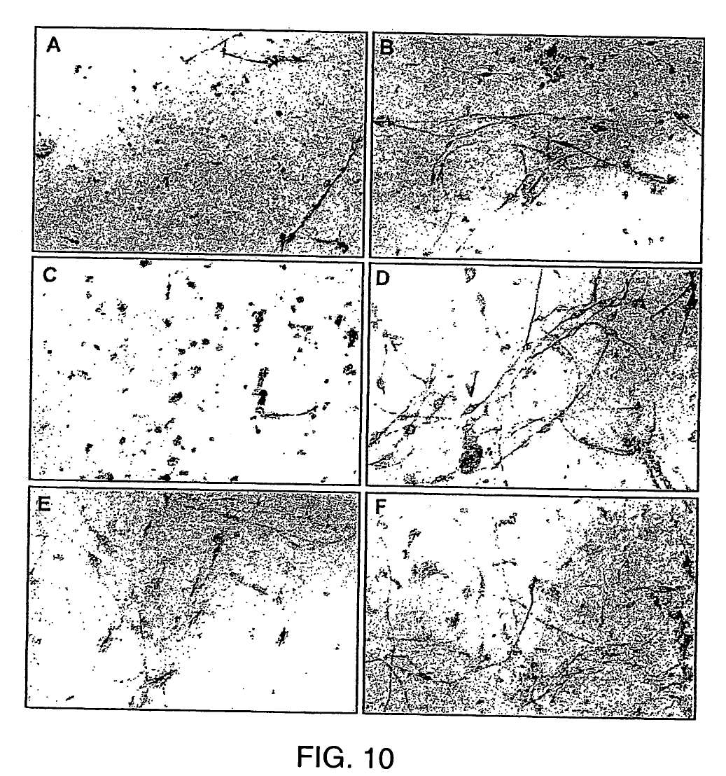

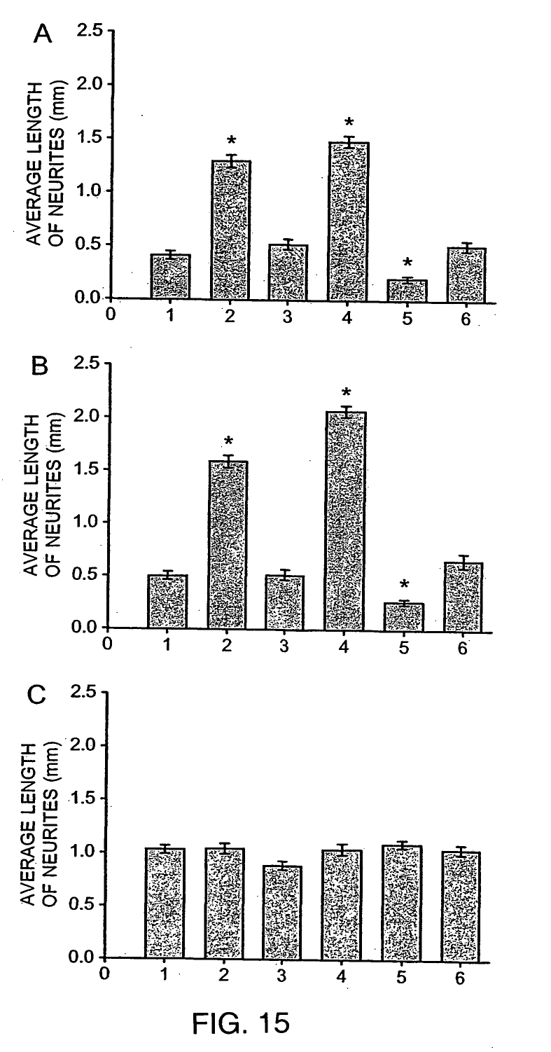

In order to eliminate any trophic effect of RA on the different populations of neurons we grew the neurons in serum free medium plus the relevant neurotrophin for a period of two days before adding 1×10⁻⁷M RA to the cultures for 3 days. Control cultures had no RA added and were maintained in neurotrophin only. There was no significant difference in the numbers of neurons cultured in the presence or absence of RA. This suggests that the effect of RA was on neurite outgrowth and not due to the selective survival of subsets of neurons under the different culture conditions used. In order to analyse neurite outgrowth the cultures were fixed after five days and stained with the monoclonal antibody NF200. Neurite length was measured by NIH image software. The experiment was repeated three times. In total approximately 120 neurons were counted in each experiment and the longest neurite was measured from each neuron from which an average neurite length was taken for each treatment. In the absence of RA the BDNF dependent neurons (FIG. 10E and FIG. 15C column 1) grew neurites whilst the NGF (FIG. 10A) and NT-3 dependent neurons (FIG. 10C) showed limited neurite outgrowth (FIGS. 15A and 15B column 1). In contrast, when RA was added to the medium there was a dramatic increase in the length and number of neurites in the NGF (FIG. 10B) and NT-3 dependent neurons (FIG. 10D) and this difference was found to be significant when the length of the neurites were compared (FIGS. 15A and 15B, columns 1 and 2). In contrast RA had no affect on neurite outgrowth of the BDNF dependent neurons (FIG. 10F and FIG. 15C columns 1 and 2).

Expression of Receptors and Response to RA by RT-PCR

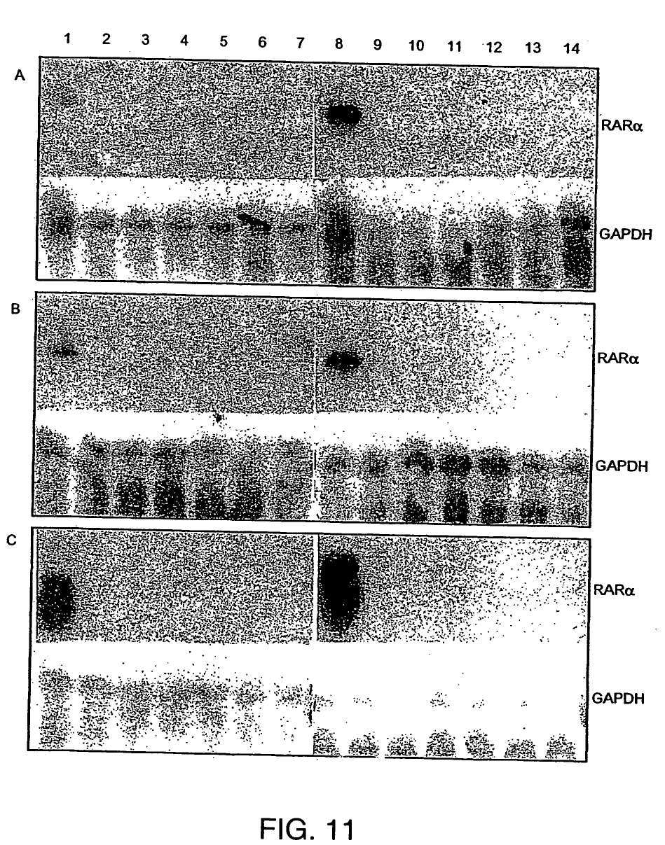

In order to identify which of the receptors are involved in neurite outgrowth semi-quantitative PCR was carried using primers against the RXRs and the individual RAR isoforms as described in the materials and methods. There was no difference in the expression of the RXRs in each of the three types of neurons cultured with or without RA. In contrast, there were variations in the RAR receptor profiles. Each of the three types of neurons expressed RARal (FIG. 11A, 11B, 11C, lane 1) which was strongly up-regulated in response to RA in the NGF (FIG. 11A, lane 8) and NT-3 (FIG. 11B, lane 8) dependent neurons and only slightly up-regulated in the BDNF dependent neurons (FIG. 11C, lane 8). It is clear from FIG. 11 that only the RARα₁ isoform is readily detectable in these DRG neurons although on over-exposure of the blots the NT-3 dependent neurons expressed the RARα₅ and RARα₇ isoforms and the BDNF dependent neurons expressed the RARα₆ and RARα₇ isoforms.

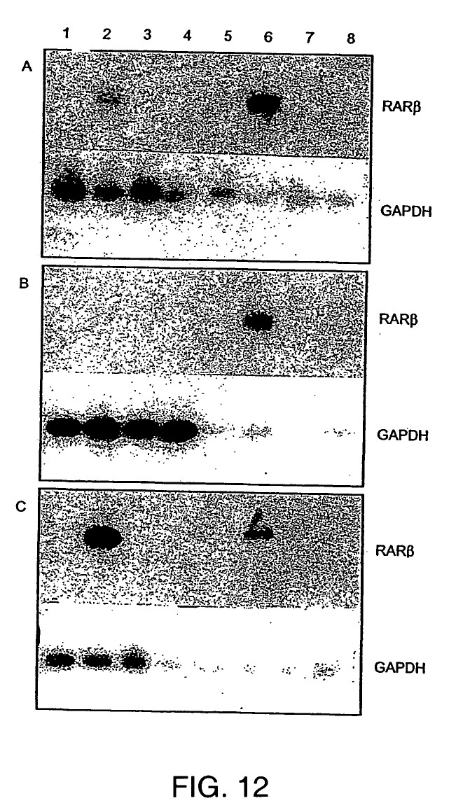

Of the four possible RARβ isoforms only the RARβ₂ isoform was detected in all three types of neuron. This isoform was strongly up-regulated by RA in the NGF (FIG. 12A, lane 6) and NT-3 dependent neurons (FIG. 12B, lane 6) but not in the BDNF dependent neurons (FIG. 12C, lane 6) as compared to the non-stimulated cultures (FIG. 12A, 12B, 12C, lane 2).

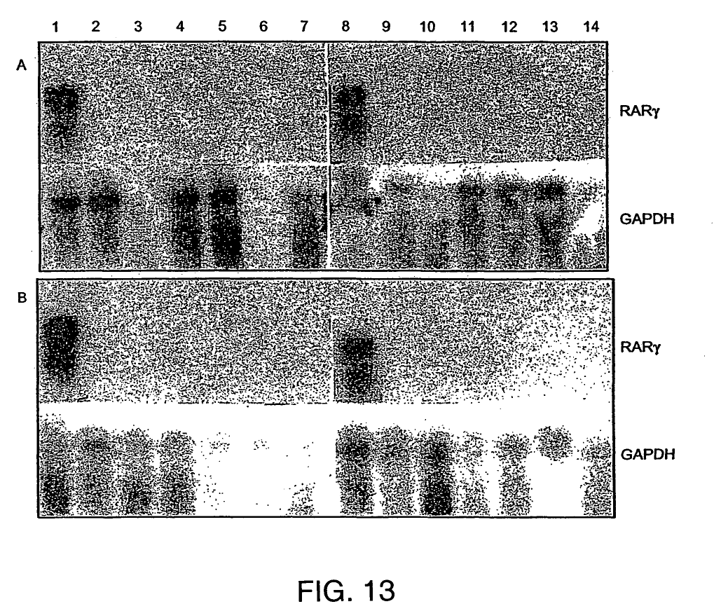

Of the seven RARy isoforms only RARγ₁ isoform was detected in the neuronal cultures and then only in the NGF (FIG. 13A, lane 1 and 8) and NT-3 dependent neurons (FIG. 13B, lane 1 and 8). No RARγ₁ was detected by RT-PCR in the BDNF dependent neurons.

Receptor Selective Analogues and Neurite Outgrowth

The above data suggested that the up-regulation of either RARcil or RARβ₂ may be responsible for the increase of neurite outgrowth observed in the NGF and NT-3 dependent neurons (FIG. 10B, 10D). It is more likely to be the RARβ₂ isoform since this receptor is not upregulated in the BDNF dependent neurons and there is no increase in neurite outgrowth when these are stimulated with RA (FIG. 10F) whereas the RARα₁ isoform is up-regulated despite a lack of neurite response to RA. In order to distinguish between these two receptors we used receptor selective synthetic retinoids which have been developed specifically to activate individual receptors. CD366 activates RARA CD2019 activates RARβ CD437 activates RARy and CD2809 activates all of the RXRs.

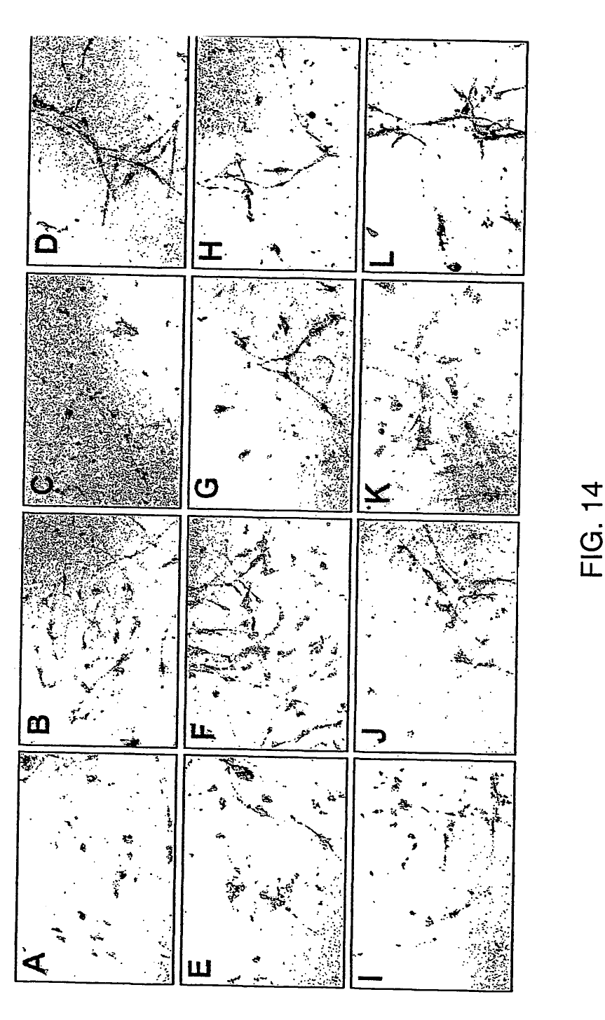

In the presence of the RARα agonist there was no significant increase in neurite outgrowth in any neuronal population (FIG. 14A, 14E, 141 and FIG. 15A, 15B and 15C columns 1 and 3).

In contrast, the RARβ agonist significantly increased neurite outgrowth in the NGF and NT-3 dependent neurons compared to non treated neurons (FIG. 14B, 14F and FIG. 15A, 15B columns 1 and 4), but did not effect neurite outgrowth in the BDNF dependent neurons (FIG. 14J and FIG. 15C columns 1 and 4). When the different neuronal populations were cultured in the presence of the RARγ agonist there was significant decrease in neurite outgrowth in the NGF and NT-3 dependent neurons (FIG. 14C, 14G and FIG. 15A, 15B columns 1 and 5) whereas neurite outgrowth still occurred in the BDNF dependent neurons (FIG. 14K and FIG. 15C columns 1 and 5). There was no significant effect on neurite outgrowth in any of the neuronal populations when they were cultured in the presence of the RXR agonist (FIG. 14D, 14H, 14L and FIG. 15A, 15B and 15C columns 1 and 6).

Therefore, in agreement with our RT-PCR data RARβ2 is required for neurite outgrowth.

Furthermore, RARγ can inhibit neurite outgrowth.

Interrelationships Between RARs

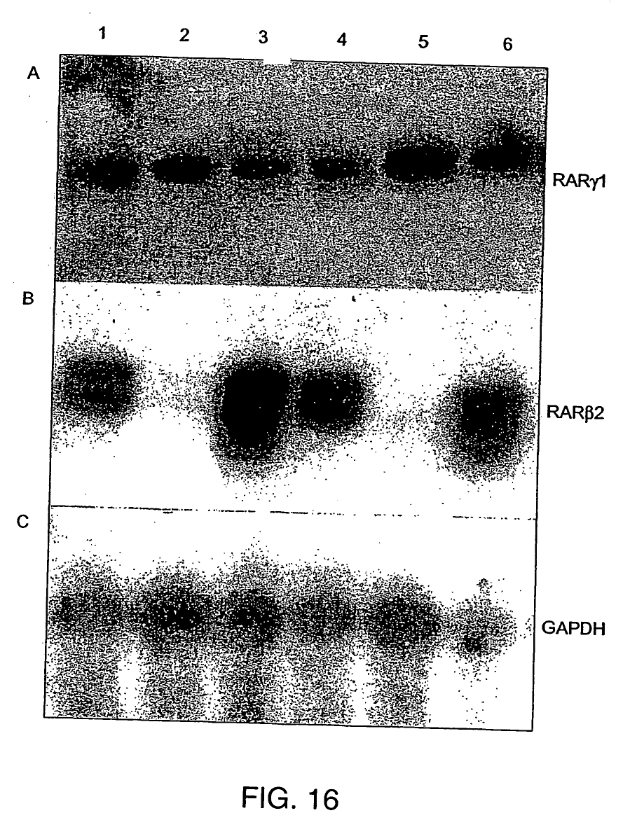

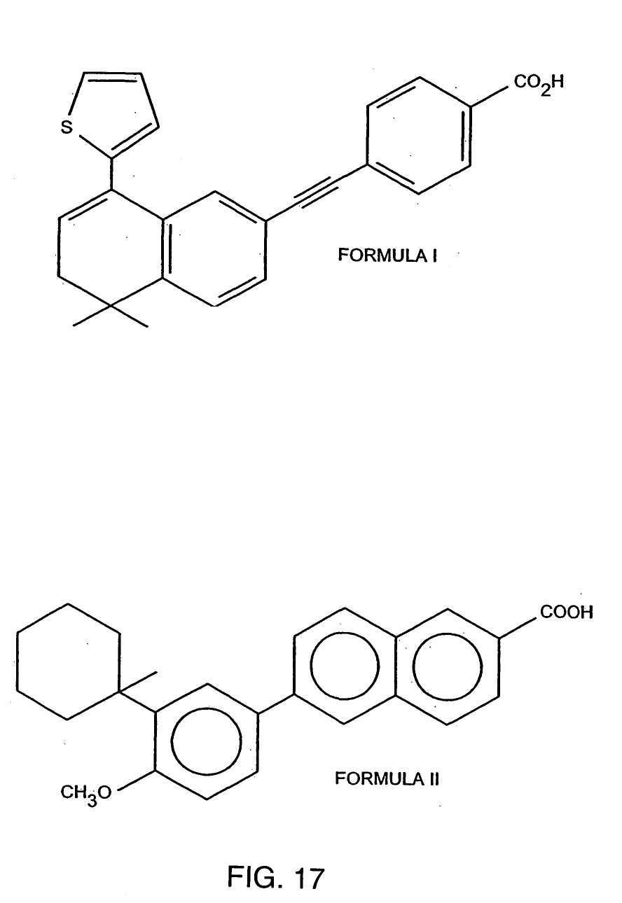

Finally, we attempted to investigate whether there were any regulative interactions between the receptors RARβ2 and RARyisince these have opposite affects on neurite outgrowth. In order to examine this we cultured NGF and NT-3 dependent neurons in serum free medium in the presence of either the RARγ agonist or the RARβ agonist and looked at the levels of receptor expression by semi quantitative RT-PCR 24 hrs. later. The RARβ agonist up-regulated the expression of RARβ₂ in both the NGF and NT-3 dependent neurons (FIG. 16B, lanes 3 and 6) compared to non-stimulated cultures (FIG. 16, lanes 1 and 4) but did not affect the expression of RARγ₁ (FIG. 15A, lanes 3 and 6). However, in the presence of the RARγ agonist, RARβ₂ is reduced in both the NGF and NT-3 dependent neurons (FIG. 16B, lanes 2 and 5) compared to non-stimulated cultures (FIG. 16B, lanes 1 and 4). The RARY agonist had no effect on the level of RARγ₁ (FIG. 16A, lanes 2 and 5). Thus RARγ₁ can regulate the expression of RARβ2.

Our results show that each of the three dorsal root ganglia neuronal populations we have isolated (NGF, NT-3 and BDNF dependent) express both a common set and a unique set of retinoid receptors. With regard to the RXRs they each express RXRα RXRβ and RXRγ and none of these were found to be directly involved in neurite outgrowth. In contrast, the neurons expressed different RARs depending on the neurotrophin used to select them. The major RAR isoforms that were common to each population were RARα₁ and RARβ₂. In addition, the NGF and NT-3 populations expressed RARγ₁ which was not expressed in the BDNF population at the time point analysed.

Only the NGF and NT-3 dependent neurons responded to RA by extending neurites whereas the BDNF dependent neurons produced neurites irrespective of the presence or absence of RA. In parallel there was a change in the RAR profile after RA addition. RARal and RARβ₂ were strongly up-regulated in the NGF and NT-3 dependent neurons whereas in the BDNF dependent neurons only the RARal was upregulated. This suggested that RARβ₂ was required for the induction of neurite outgrowth and to confirm this observation we used receptor selective agonists.

The development of receptor selective agonists has provided an extremely valuable tool to begin to examine the role of individual receptors in any particular biological process. We showed here that only the RARβ agonist, CD2019, mimiced the effect of RA by inducing neurite outgrowth in NGF and NT-3 neurons thus confirming our RT-PCR results.

We also observed that the RARγ agonist caused a decrease in neurite outgrowth of the NGF and NT-3 dependent neurons. In an attempt to show whether this was associated with the RARβ₂ expression we examined whether the RAR agonists had any effect on receptor expression. The RARβ agonist upregulated the expression of RARβ₂ but had no effect on the expression of RARγ1. In contrast whilst the RARγ agonist had no effect on the expression of RARγ1 it did down-regulate the level of RARβ₂ expression, this phenomenon may also be a prelude to neurite outgrowth. This suggests that the RARβ transcript can be regulated by RARγ/RXR heterodimers. The lack of increase in neurite outgrowth in response to RA of the BDNF dependent neurons also suggests that in this type of neuron that RARβ may be regulated differently to RARβ in the NGF and the NT-3 dependent neurons at the embryonic stage studied.

Our results suggest that it is the activation of the RAR pathway that is responsible for neurite outgrowth, since a RXR agonist which activates RXR/orphan receptors had no effect on neurite outgrowth whereas the RARβ agonist which activates RAR/RXR heterodimers increased the amount of neurite outgrowth. This suggest that if NGF acts via the RXR/orphan receptor pathway by utilising for example NGFI-B then it is not, in these embryonic stages, directly responsible for neurite outgrowth, rather it may be required for neuron survival. Interestingly in the adult the contrast seems to be true. NGF is not required for neuron survival but it is required for neurite outgrowth (Lindsay, 1988). Thus there may be different mechanisms for neurite outgrowth in developing and adult regenerating neurites. However, there is a absolute requirement for RA in neurite outgrowth during development. In the vitamin A deficient quail the neural tube fails to extend neurites into the periphery (Maden et al. 1996; Maden et al., 1998).

The differential response of these neurons to RA and the receptor agonists may have some significance for embryonic and adult tissues which require retinoids for their development and/or survival. In order to activate different RAR/RXR and RXR/orphan receptor combinations there may be different retinoids present in the tissues. Some support for this view is provided by the fact that there are numerous RA generating enzymes which show localised expression during development (McCaffery et al., 1992; Drager & McCaffery, 1995; Godbout et al., 1996; Neiderreither et al., 1997; Ang & Duester, 1997) and each of these enzymes could make different retinoids. Several novel retinoids have so far been discovered, for example 5,6-epoxyretinoic acid, which is found in the intestine (McCormick et al., 1978), 4-oxo-retinol which is the biologically active metabolite that is responsible for the differentiation of murine embryonic F9 cells (Achkar et al., 1996) and 14-hydroxy-4, 14-retroretinol which is found in B lymphocytes (Buck et al., 1991).

Therefore, the embryo may be able to regulate the amount of neurite outgrowth by synthesising different retinoids. By activating RARβ₂RXR heterodimers neurite outgrowth could occur whereas by activating RARγ/RXR heterodimers neurite outgrowth could be stopped. In addition the amount of neurite outgrowth could be regulated by the amount of retinoic acid. For example in the developing mouse spinal cord there are high concentrations of retinoids in the brachial and lumbar enlargements (McCaffery & Drager, 1994). This may be an intrinsic requirement for innervation of the extremities of the limb where the neurites have to travel large distances to reach their final targets, whereas in the thoracic region where the concentration of retinoids are lower such extensive neurite outgrowth would not be required.

### Example 4

### Gene Transfer to Non-Dividing Cells

This example demonstrates gene transfer to dorsal root ganglion, ie. gene transfer to non-dividing neuronal cells, by the equine infectious anaemia virus vector, pONY8Z.

The EIAV vector, pONY8Z, is made by transient co-transfection of 293T human embryonic kidney cells with either pONY8Z plasmid, pONY3.1 and an envelope expression plasmid, pRV67 (which encodes the vesicular stomatitis virus protein G, VSV-G) using the calcium phosphate precipitation method.

The pONY8.0Z plasmid is a variant of pONY4.0Z (see sequence listing) but it does not express any EIAV sequence. The first two ATG sequences in gag are changed to ATTG and the accessory genes Tat, S2 and Rev are either deleted or stop codons inserted in their open reading frames. The env start codon is also removed.

Twenty four hours before transfection the 293T cells are seeded at 3.6×10⁶ cells per 10 cm dish in 10 ml of DMEM supplemented with glutamine, non-essential aminoacids and 10% foetal calf serum. Transfections are carried out in the late afternoon and the cells are incubated overnight prior to replacement of the medium with 6 ml of fresh media supplemented with sodium butyrate (5 mM). After 7 hours the medium is collected and 6 ml of fresh unsupplemented media added to the cells. The collected medium is cleared by low speed centrifugation and then filtered through 0.4 micron filters.

Vector particles are then concentrated by low speed centrifugation (6,000 g, JLA10.500 rotor) overnight at 4° C. and then the supernatant is poured off, leaving the pellet in the bottom of the tube. The following morning the remaining tissue culture fluid is harvested, cleared and filtered. It is then placed on top of the pellet previously collected and overnight centrifugation repeated. After this the supernatant is decanted and excess fluid is drained. Then the pellet is resuspended in phosphate-buffered saline to 1/1000 of the volume of starting supernatant. Aliquots are then stored at −80° C.

Dorsal root ganglia are prepared for transduction with pONY8Z vector. Adult rats (eight months old) are sacrificed, and the dorsal root ganglia (DRG) removed. This is accomplished by first dissecting away the spinal cord, and then removing the DRG from the bony crevices on the inner surface of the vertebrae (ie. from crevices in the intra-vertebral space or canal which carries the spinal cord). The whole explanted ganglia are then placed in a cellogen matrix medium, said medium as described in Examples 1 and 2 above.

Transduction is carried out by injecting 3ul of the 1000× concentrated vector particles, produced as described above, into the DRG explant. This is accomplished using a fine chromatography needle.

pONY8Z carries a β-galactosidase gene, expression of which is driven by the CMV promoter. Transduction is assessed by X-gal staining, said staining as described in (Lim, F., Hartley, D., Starr, P., Song, S., Lang, P., Yu, L., Wang, Y. M. & Geller, A. I. Use of defective herpes-derived plasmid vectors. Meth. Mol. Biol. 62, 223-232 (1997)).

Thus, expression of nucleic acid sequences using EIAV vectors according to the present invention is demonstrated.

The vectors may also be produced according to the invention using different proteins capable of pseudotyping EIAV, such as Rabies G or variants of Rabies G (WO99/61639) (using pRabG plasmid or a variant such as pSA91RbG plasmid) or VSV-G (using pRV67 plasmid) as described above. The method of production is as described above, except that a VSV-G expression plasmid is replaced with an expression plasmid for Rabies G protein.

DRGs are prepared and infected as described above.

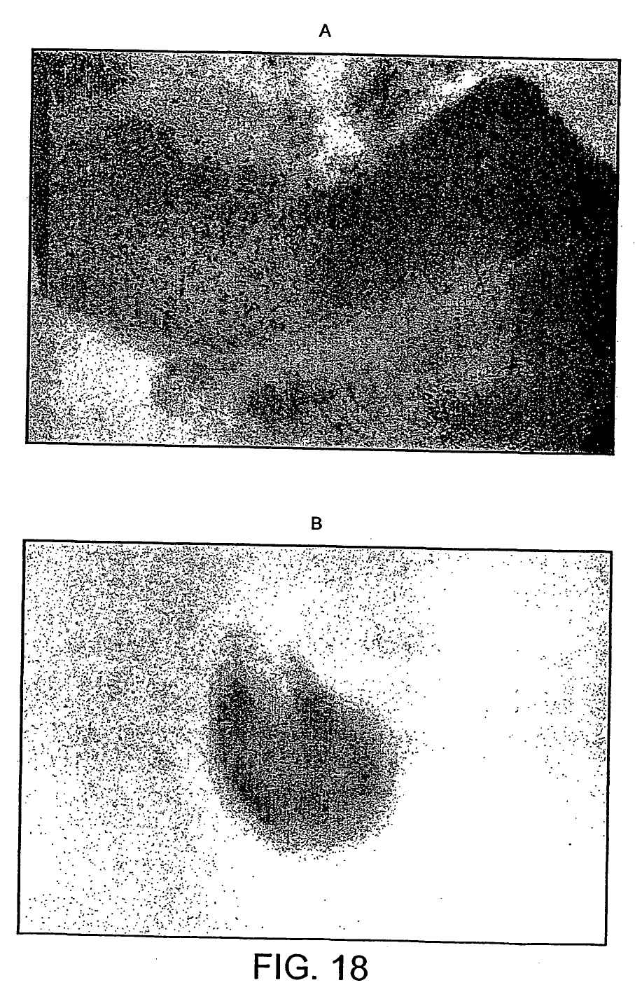

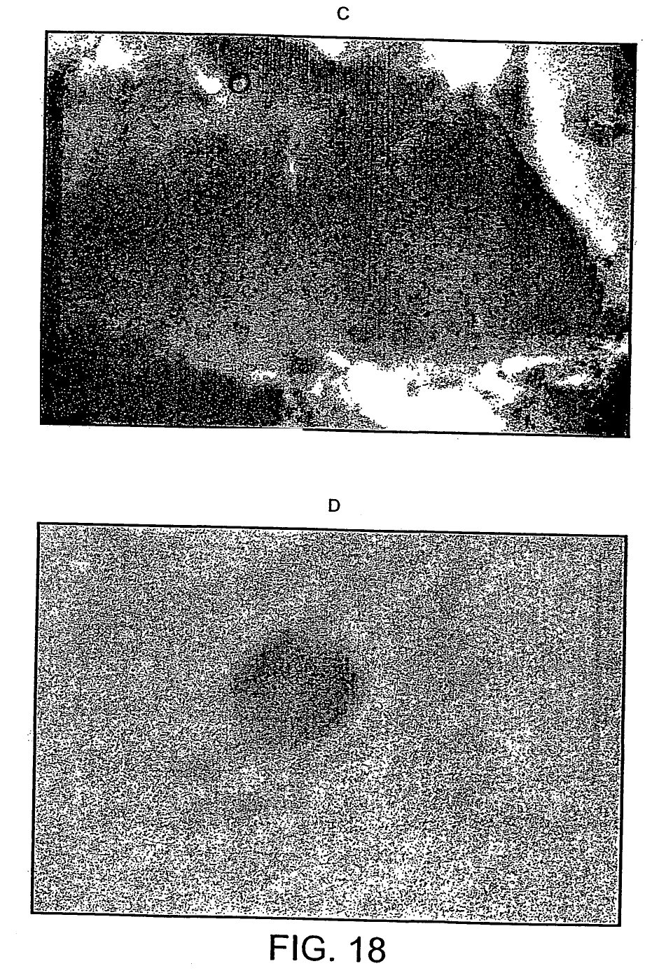

X-gal staining is performed as described above (Lim, F., Hartley, D., Starr, P., Song, S., Lang, P., Yu, L., Wang, Y. M. & Geller, A. I. Use of defective herpes-derived plasmid vectors. Meth. Mol. Biol. 62, 223-232 (1997)). Typical results are shown in FIG. 18, which shows four photomicrographs.

Thus, it is demonstrated the vectors of the present invention are capable of producing expression of a nucleic acid of interest in non-dividing cells. Further, it is clearly demonstrated that expression of vector sequences according to the present invention may be produced in non-dividing adult neuronal cells.

### Example 5

### Production of EIAV Vector Genome Expressing RARβ2

A fragment of DNA encoding the retinoic acid receptor β2 is amplified by the polymerase chain reaction from a suitable template for RARβ2 such as cDNA produced from Trizol-prepared RNA as described in Example 2, or alternatively any nucleic acid molecule comprising RARβ2 such as described in Accession Number NM_{—}000965. The oligonucleotide primers used are:

RARβ2 FWD:
5′ CAG TAC ccg.cgg GCC ACC ATG TTT GAC TGT ATG GAT
GTT CTG 3′
RARβ2 REV:
5′ CAG TAC ctg cag.ATC ATT GCA CGA GTG GTG ACT
GAC T 3′

The oligonucleotide primers contain SacII and PstI recognition sites respectively in order to facilitate cloning into the EIAV vector genome. In addition a Kozak sequence (GCCACC) is introduced upstream of the ATG initiation codon of the RARβ2 gene in RARβ2 FWD to improve the efficiency of translational initiation and the termination codon and context (in RARβ2 REV) is changed to UGAA which has been shown to be the most efficient termination signal in eukaryotes.

The resultant 1,378 bp PCR product encoding RARβ2 is digested with SacII and PstI and ligated into the EIAV vector genomes, pONY9Z 5′POS MIN or pONY9Z 3′POS MIN prepared for ligation by digestion with SacII and SbfI. These enzymes cut the DNA on either side of the LacZ reporter gene. Vector plasmids pONY9Z 5′POS MIN or pONY9Z 3′POS MIN are derivatives of pONY8Z constructed as described in the following paragraphs.

Construction of pONY9Z 5′POS MIN and pONY9Z 3′POS MIN.

The presence of a sequence termed the central polypurine tract (cPPT-see Stetor et al. Biochemistry. 1999 Mar. 23;38(12):3656-6) may improve the efficiency of gene delivery to non-dividing cells. This cis-acting element is located in the polymerase coding region element and can be obtained as a functional element by using PCR amplification using any plasmid which contains the EIAV polymerase coding region (for example pONY3.1) as follows. The PCR product includes the central polypurine tract and the central termination sequence (CTS). The oligonucleotide primers used in the PCR reaction are:

EIAV cPPT POS:
CAGGTTATTCTAGAGTCGACGCTCTCATTACTTGTAAC
EIAV cPPT NEG:
CGAATGCGTTCTAGAGTCGACCATGTTCACCAGGGATTTTG

Recognition sequences for XbaI and SalI are in italic and bold respectively and facilitate insertion into the pONY8Z backbone.

Before insertion of the cPPT/CTS PCR product prepared as described above, pONY8Z is modified to remove the CTS which already is present the pONY8Z vector. This is achieved by subcloning the SalI to ScaI fragment encompassing the CTS and RRE region from pONY8Z into pSP72, prepared for ligation by digestion with SalI and EcORV. The CTS region is then removed by digestion with KpnI and PpuMI, the overhanging ends ‘blunted’ by T4 DNA polymerase treatment and then the ends religated. The modified EIAV vector fragment is then excised using SalI and NheI and ligated into pONY8Z prepared for ligation by digestion with the same enzymes. This new EIAV vector is termed pONY8Z del CTS.

pONY8Z del CTS has unique XbaI and SalI sites which are located immediately upstream and downstream of the CMV-LacZ unit, respectively. The cPPT/CTS PCR product is digested with either of these enzymes and then ligated into pONY8Z del CTS prepared for ligation by digestion with either XbaI or SalI. Ligation into these sites results in plasmids with the cPPT/CTS element in either the positive or negative senses. Clones in which the cPPT/CTS is in the positive sense (functionally active) at either the 5′ or 3′-position are termed pONY9Z 5′POS and pONY9Z 3′POS, respectively.

The safety profile of the EIAV vector can be improved by arranging for the integrated vector to have functionally inactive LTR's. Such vectors are termed SIN (Self Inactivating Vectors). In this way the only transcription events associated with the presence of the vector following transduction are those from the internal promoter. In the pONY8 and pONY9 series of vectors the internal promoter is CMV however other promoters, such as tissue specific promoters, can be used. The SIN configuration is created by making a deletion in the U3 region of the 3′LTR using PCR-based techniques as follows. The template for amplification is pONY8Z, and the primers used for amplification are:

MIN FOR:
CACCTAGCAGGCGTGACCGGTGG
MIN REV:
CCTACCAATTGTATAAAACCCCTCATAAAAACCCCAC

The forward primer binds just 5′ of a unique NspV site in pONY8Z and the reverse primer binds to the 5′ end of the U3 region and has MunI site (in bold) at the 5′end. Thus the PCR product includes sequences corresponding to the second exon of EIAV REV and extends through the 3′polypurine tract to include the 5′ 26 nucleotides of U3. The PCR product is digested with NspV and MunI and ligated into either pONY9Z 5′POS or pONY9Z 3′POS prepared for ligation using the same enzymes. The sequence of the resulting plasmid is confirmed by sequence analysis and the plasmids termed pONY9Z 5′POS MIN or pONY9Z 3′POS MIN.

Production of pONY9-RARβ2 Vector Preparations

Vector preparations are made by transient co-transfection of 293T human embryonic kidney cells with either pONY9 5′-RARβ2 or pONY9 3′-RARβ2 plasmid, pONY3.1 and an envelope expression plasmid such as pRV67 (which encodes VSV-G), pRabG or derivatives of pRabG (Rabies virus G protein) or expression plasmids encoding other proteins capable of pseudotyping EIAV.

Alternatively the pE SYN GP cassette, which encodes the EIAV gag/pol protein but which is optimised for expression in human cells by altering the codon usage, can be used instead of pONY3.1. This cassette can be expressed in any conventional eukaryotic gene expression vector. Alternatively, pESDSYNGP which has a splice donor in the leader can be used. When transfections are carried out in this way, higher yields are obtained if a fourth plasmid encoding EIAV REV is also included in the transfection.

Transfections, harvesting and concentration of the vector particles are carried out as described above for pONY8Z.

Assessement of pONY9-RARβ2 Vector Preparations

pONY9 RARβ2 assessment of titre is made by measuring properties of the vector preparation: the reverse transcriptase (RT) activity and the incorporation of vector RNA into particles can be used together or independently in order to estimate vector titre. These are then related to those of other vector preparations, for example pONY8Z, of known biological titre, giving an estimate of the titre of the vector preparation being tested.

The amount of RT activity is assessed by performance enhanced reverse transcriptase assay (PERT). This assay has been previously described by Lovatt et al., (1999), J. Virological Methods, 82, 185-200 using brome mosaic virus RNA as template for the RT. In this Example, MS2 bacteriophage RNA is used instead of the brome mosaic virus RNA described therein. Briefly this assay works as follows: RT activity is released from the vector particles present in the preparation by mild detergent treatment and used to synthesise cDNA to RNA from MS2 bacteriophage. The MS2 RNA and primer are present in excess therefore the amount of cDNA synthesised is proportional to the amount of RT activity released. The MS2 cDNA is then quantitated by PCR methods using an ABI PRISM 7700 Sequence Detector. The value obtained enables comparison with the standard pONY8Z vector preparation, and hence titre to be assessed. The details of the assay are as follows:

The PERT assay uses real time quantitative RT-PCR technology to detect a specific PCR product from MS2 RNA and the retroviral reverse transcriptase present in the viral particles (in this case EIAV RT). Briefly, the viral particles are disrupted by mixing 1:1 volumes of viral vector stocks and disruption buffer (40 mM Tris-HCl pH7.5, 50 mM KCl, 20 mM DTT and 0.2% NP-40). Serial dilutions of the disrupted particles are carried out prior to adding them to the RT-PCR TaqMan reaction mix (Perkin-Elmer). The reaction mix contains 1/10th volume of disrupted viral particles, 300 nM PERT forward primer, 300 nM PERT reverse primer, 150 nM PERT probe, 80 mg/ml MS2 RNA. The RT-PCR conditions are as follows: 48° C. for 30 min; 95° C. for 10 min; then 40 cyles of, 95° C. for 15sec and 60° C. for 1 min. The data is analysed using the TaqMan software (Perkin-Elmer).

PERT pimers are derived using the primer/probe prediction programs on the TaqMan using MS2RNA Acc. No. J02467 as input;

NEGATIVE SENSE PRIMER, FOR REVERSE TRANSCRIPTASE
STEP =
5′-CACAGGTCAAACCTCCTAGGAATG
PLUS SENSE PRIMER =
5′ TCCTGCTCAACTTCCTGTCGA
PROBE =
5′ FAM-CGAGACGCTACCATGGCTA-(TAMRA)p3′

The use of MS2 RNA as template in the F-PERT assay is as described in Arnold et al (BioTechniques, (1998), vol25, 98-106).

Assessment of Titre Via Measurement of Packaged Vector RNA

Detection of the packaging signal is accomplished by monitoring the incorporation of vector RNA into particles which is quantified using the packaging signal assay as follows. The RNA content of the viral preparations is estimated by RT-PCR comparing to a pONY8G vector preparation of known biological titre (see above). Vector RNA is isolated from the vector stocks using a Qiagen RNA isolation kit (Qiagen) and then DNAse treated using RNAse free DNAse (Ambion). Serial dilutions of the RNA are used as template in the RT-PCR reaction. Two TaqMan (Perkin-Elmer) reaction mixes are prepared, +RT and −RT, containing 1/10th volume of RNA template and the specific forward and reverse primers and probe. The RT-PCR conditions are as follows: Hold, 48° C. for 30 min; hold, 95° C. for 10 min; forty cycles, 95° C. for 15sec and 60° C. for Imin. The data was analysed using the TaqMan software (Perkin-Elmer).

NEGATIVE SENSE PRIMER, FOR REVERSE TRANSCRIPTASE
STEP =
5′-accagtagttaatttctgagacccttgta
PLUS SENSE PRIMER =
5′ ATTGGGAGACCCTTTGACATT
PROBE =
5′ FAM-CACCTTCTCTAACTTCTTGAGCGCCTTGCT-(TAMRA)p3′

(This set of primers detects vector genome, but not wild type gag/pol.)

The biological titre of the vector is related to the amount of vector RNA packaged in virions and can be assessed using quantitative RT-PCR using the ABI PRISM 7700 Sequence Detector. The primers and probe for the reaction bind to the packaging signal region of the vector.

Thus, it is demonstrated that vector particles for the delivery of RARβ2 may be produced according to the invention.

### Example 6

### Transfer of Genetic Material to Non-Dividing Cells

This example demonstrates gene transfer to dorsal root ganglion (DRG), i.e., gene transfer to non-dividing neuronal cells, by the equine infectious anaemia virus vector, pONY8.0Z.

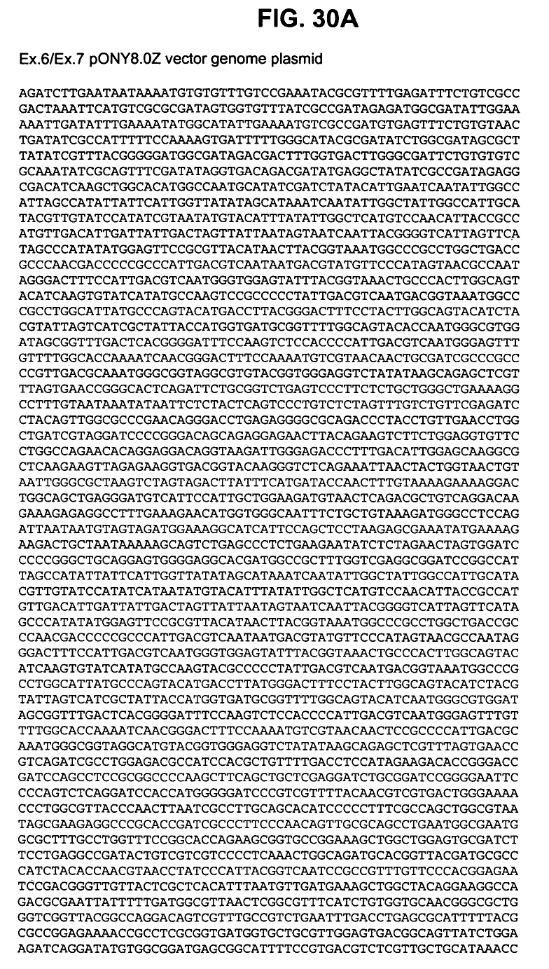

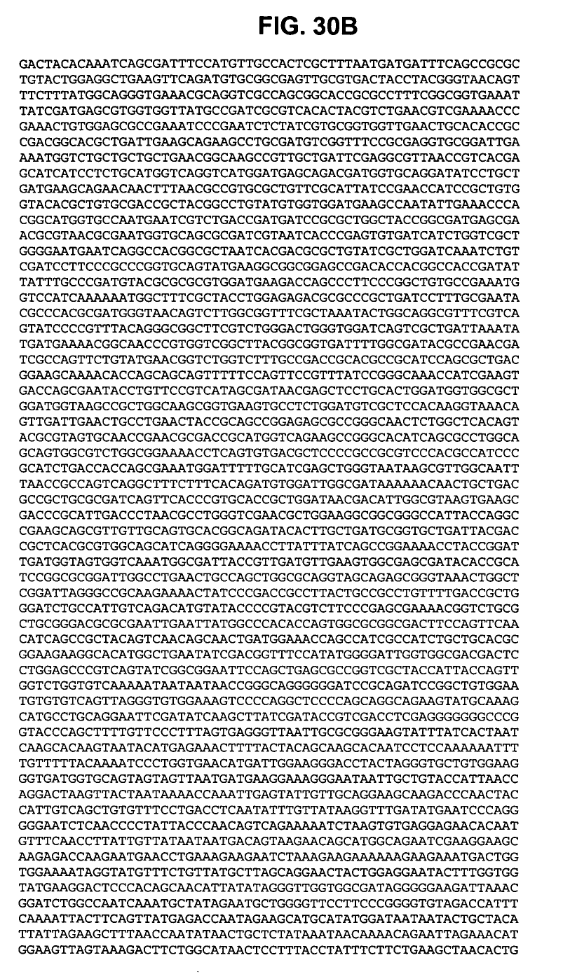

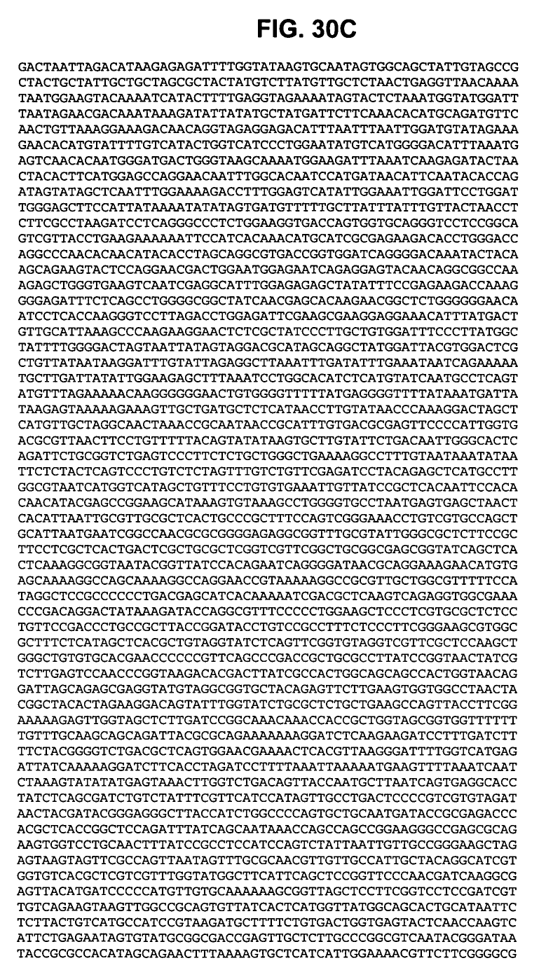

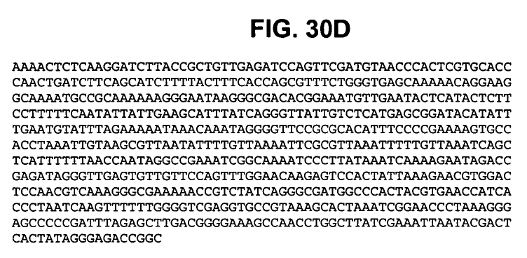

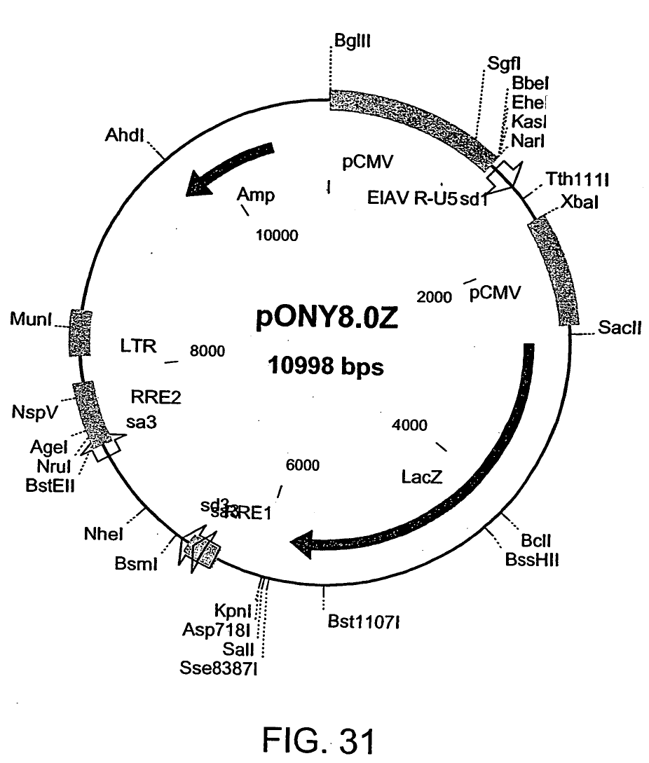

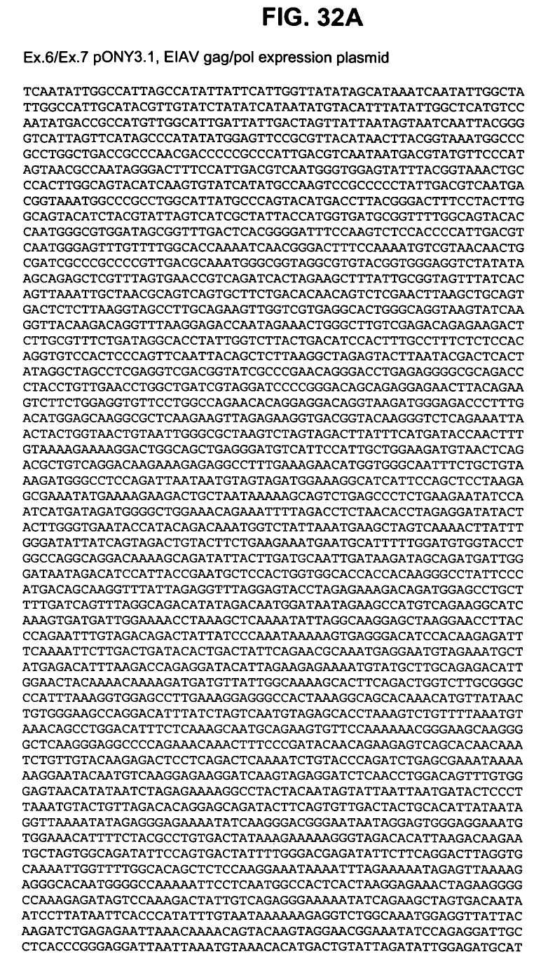

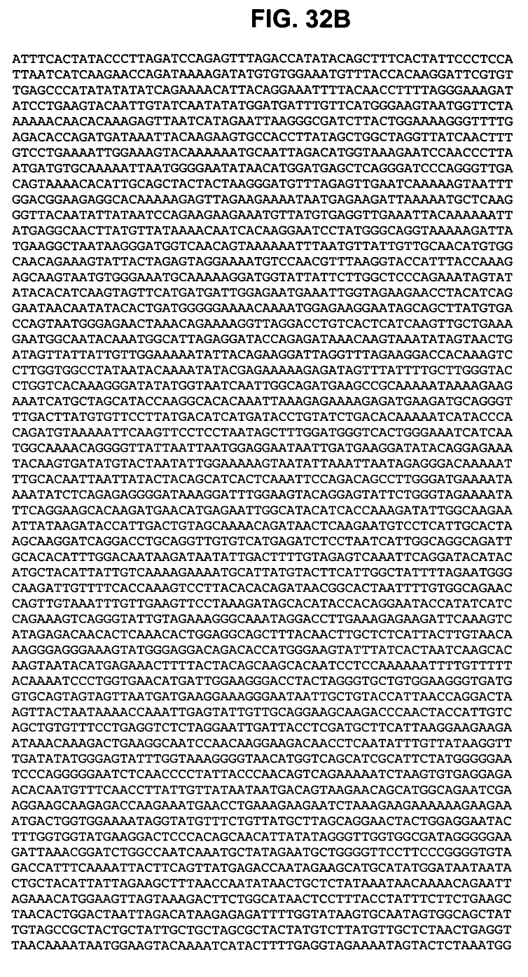

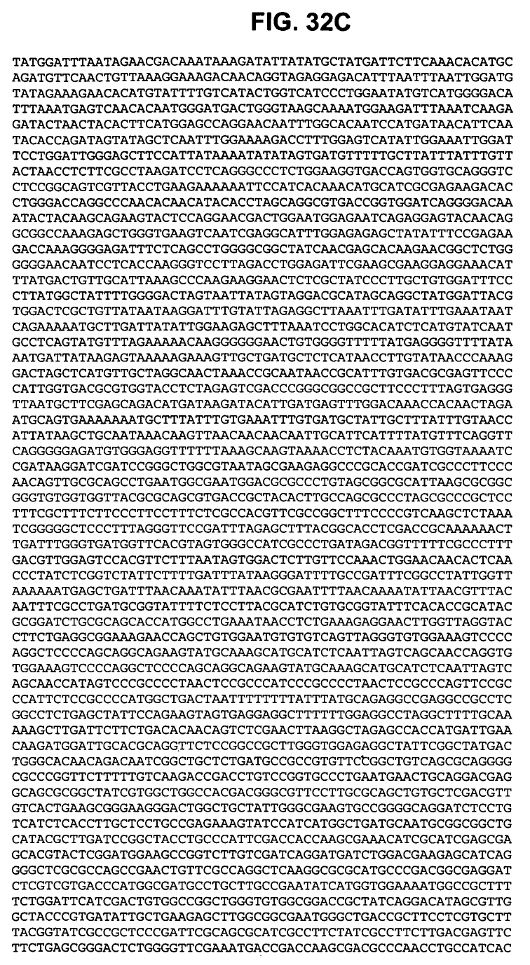

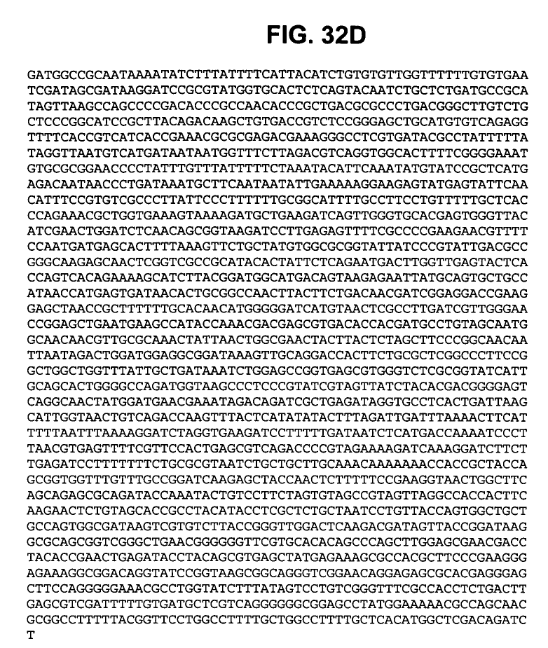

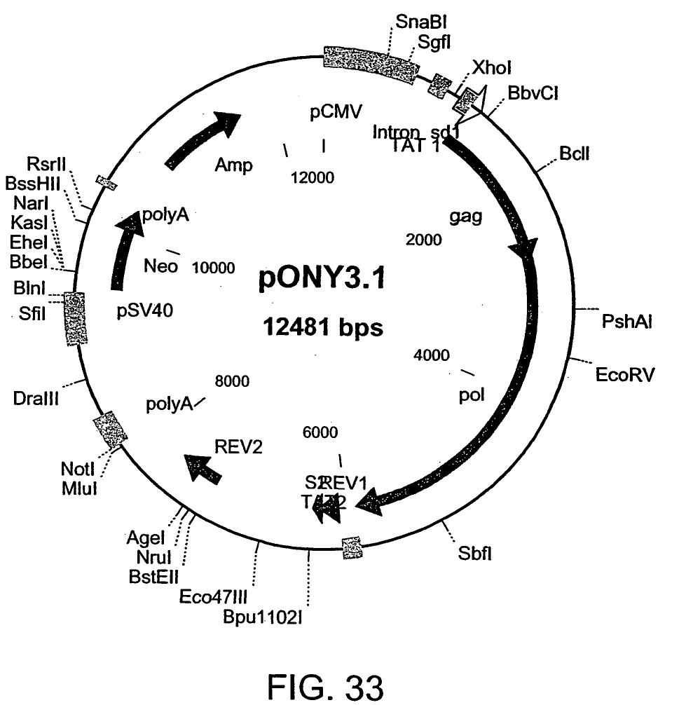

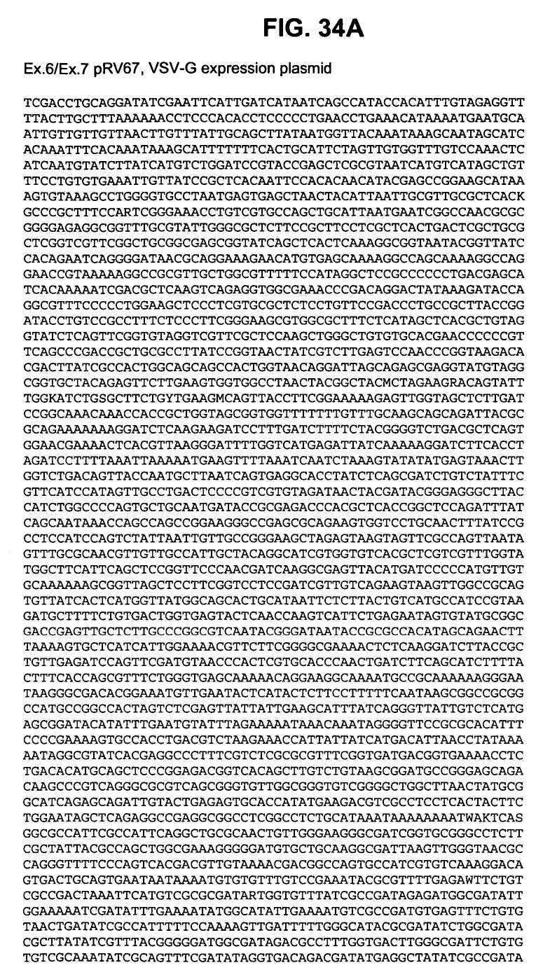

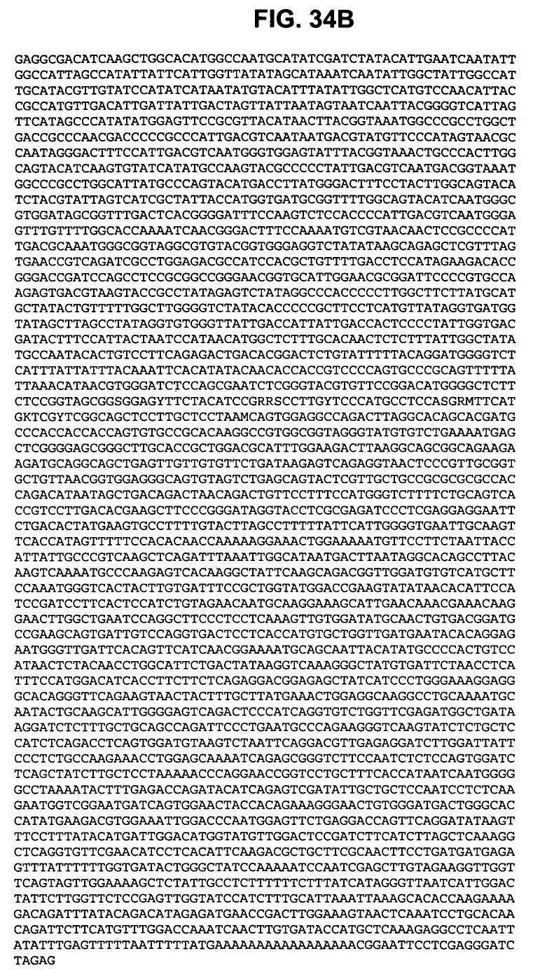

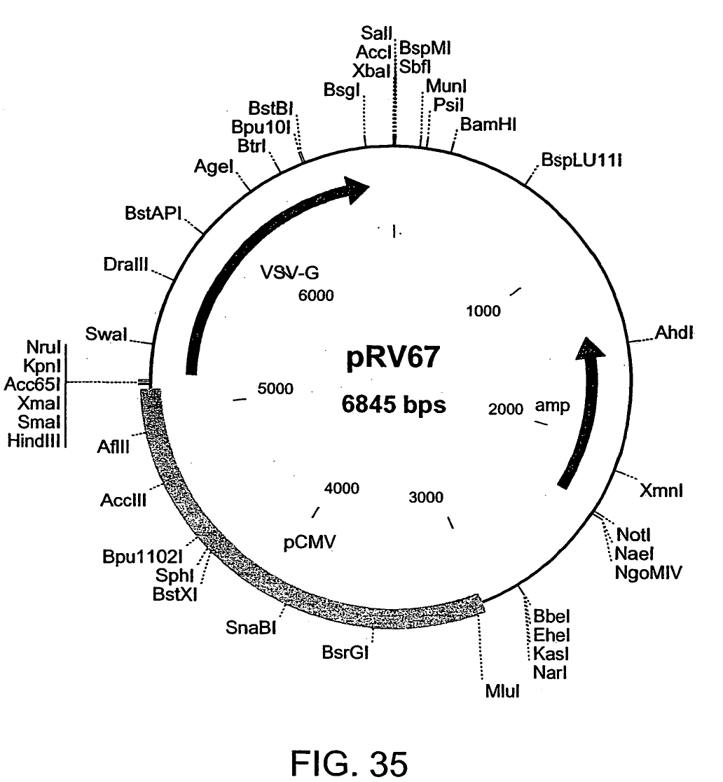

The EIAV vector, pONY8.0Z is made by transient co-transfection of HEK 293T human embryonic kidney cells with pONY8.0Z vector genome plasmid (FIGS. 30 and 31), pONY3.1 (FIGS. 32 and 33) (WO99/32646) and an envelope expression plasmid, pRV67 (FIGS. 34 and 35) (WO99/61639)(which encodes the vesicular stomatitis virus protein G, VSV-G) using the calcium phosphate precipitation method, as described below.

Vectors may also be produced according to the invention using different proteins capable of pseudotyping EIAV, such as Rabies G or variants of Rabies G (WO99/61639) (using pRabG plasmid or a variant such as pSA91RbG plasmid) or VSV-G (using pRV67 plasmid) as described above. The method of production is as described above, except that a VSV-G expression plasmid is replaced with an expression plasmid for Rabies G protein.

pONY8.0Z is derived from pONY4.0Z (WO/9932646) by introducing mutation(s) as follows:

Mutation(s) are introduced which prevent expression of tat. In the present Example, this is accomplished by an 83 nt deletion in the exon 2 of tat.

Mutation(s) are introduced which prevent S2 ORF expression. In the present Example, this is accomplished by a 51 nt deletion.

Mutation(s) are introduced which prevent REV expression. In the present Example, this is accomplished by deletion of a single base within exon 1 of rev.

Mutation(s) are introduced which prevent expression of the N-terminal portion of gag. In the present Example, this is accomplished by insertion of T in ATG start codons, thereby changing the sequence to ATTG from ATG. With respect to the wild type EIAV sequence Acc. No. U01866 these correspond to deletion of nt 5234-5316 inclusive, nt 5346-5396 inclusive and nt 5538. The insertion of T residues is after nt 526 and 543.

The method of vector production by calcium phosphate-mediated transfection is as follows. Twenty four hours before transfection the HEK 293T cells are seeded at 3.6×10⁶ cells per 10 cm dish in 10 ml of DMEM supplemented with glutamine, non-essential aminoacids and 10% foetal calf serum. Transfections are carried out in the late afternoon and the cells are incubated overnight prior to replacement of the medium with 6 ml of fresh media supplemented with sodium butyrate (5 mM). After 7 hours the medium is collected and 6 ml of fresh unsupplemented media added to the cells. The collected medium is cleared by low speed centrifugation and then filtered through 0.45 micron pore-size filters.

Vector particles are then concentrated by low speed centrifugation (6,000g, JLA10.500 rotor) overnight at 4° C. and then the supernatant is poured off, leaving the pellet in the bottom of the tube. The following morning the remaining tissue culture fluid is harvested, cleared and filtered. It is then placed on top of the pellet previously collected and overnight centrifugation repeated. After this the supernatant is decanted and excess fluid is drained. Then the pellet is resuspended in phosphate-buffered saline to 1/1000 of the volume of starting supernatant. Aliquots are then stored at −80° C.

Dorsal root ganglia are prepared for transduction with pONY8.0Z vector. Adult rats (eight months old) are sacrificed, and the DRG removed. This is accomplished by first dissecting away the spinal cord, and then removing the DRG from the bony crevices on the inner surface of the vertebrae (i.e., from crevices in the intra-vertebral space or canal which carries the spinal cord). The whole explanted ganglia are then placed in a cellogen matrix medium, said medium as described in Examples 1 and 2 above. Sections of the spinal cord were also cultured in cellogen matrix medium in DMEM/F12 medium with 5% Foetal Calf Serum (FCS).

The ability of pONY8.0Z to transduce either the spinal cord or DRG explants was assessed by injecting 3 μl of the 1000× concentrated pONY8.0Z vector particles, produced as described above, into the explants. This was accomplished using a fine chromatography needle. After 5 days, they were stained for β-galactosidase expression. pONY8.0Z carries a β-galactosidase gene, expression of which is driven by the CMV promoter. Therefore transduction is easily assessed by X-gal staining, said staining as described in (Lim, F., Hartley, D., Starr, P., Song, S., Lang, P., Yu, L., Wang, Y. M. & Geller, A. I. Use of defective herpes-derived plasmid vectors. Meth. Mol. Biol. 62, 223-232 (1997)).

Thus, expression of nucleic acid sequences using EIAV vectors according to the present invention is demonstrated. Furthermore, it is demonstrated the vectors of the present invention are capable of producing expression of a nucleic acid of interest in non-dividing cells. Further, it is clearly demonstrated that expression of vector sequences according to the present invention may be produced in non-dividing adult neuronal cells.

### Example 7

### Construction and Manufacture of Eiav Vector Genome Expressing Retinoic Acid Recptor β2 (RARβ2)

A fragment of DNA encoding the retinoic acid receptor β2 is amplified by the polymerase chain reaction from a suitable template for RARβ2 such as cDNA produced from Trizol-prepared RNA as described in Example 2, or alternatively any nucleic acid molecule comprising RARβ2, such as described in Genbank Acc. No. S56660. Two versions were made: one in which a wild type RAR-β2 sequence was constructed and one in which it was preceded by the ‘FLAG’ epitope tag (Immunex Corporation). The FLAG sequence allows easy identification RARβ2 expression. The oligonucleotide primers used were:

EX7 RARβ2 FWD:
5′ACTGccg.cgg GCC ACC ATG TTT GAC TGT ATG GAT GTT
CTG TC3′

EX7 RARβ2 FLAG FWD:
5′ACTGccg.cgg GCC ACC ATG GACTACAAGGACGACGATGACA
AG TTT GAC TGT ATG GAT GTT CTG TC3′

EX7 RARβ2 REV:
5′ACTGGCGGCCGCTCACTGCAGCAGTGGTG3′

The oligonucleotide forward (FWD) and reverse (REV) primers contain SacII and NotI recognition sites, respectively, in order to facilitate cloning into the EIAV vector genome. In addition a Kozak sequence (GCCACC) was introduced upstream of the ATG initiation codon of the RARβ2 or FLAG RARβ2 gene in the forward (FWD) primers to improve the efficiency of translational initiation.

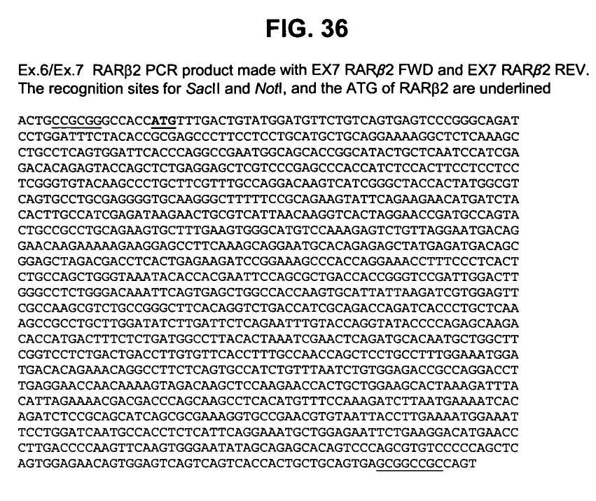

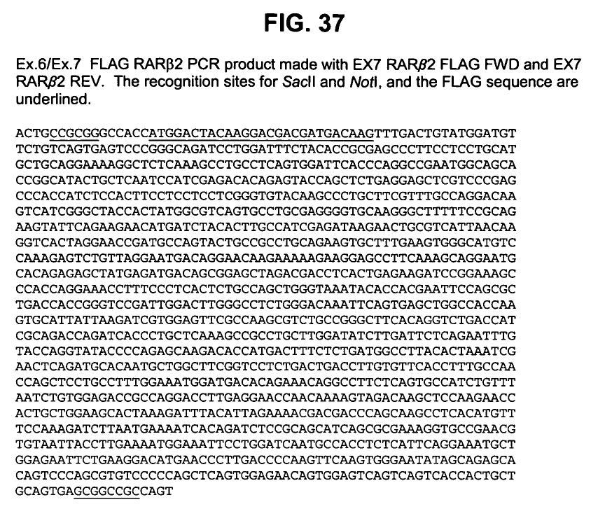

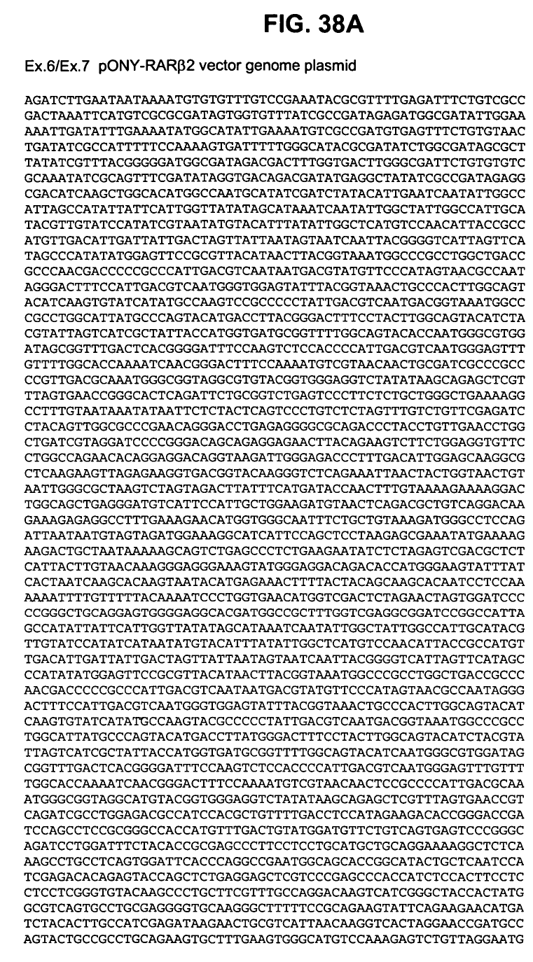

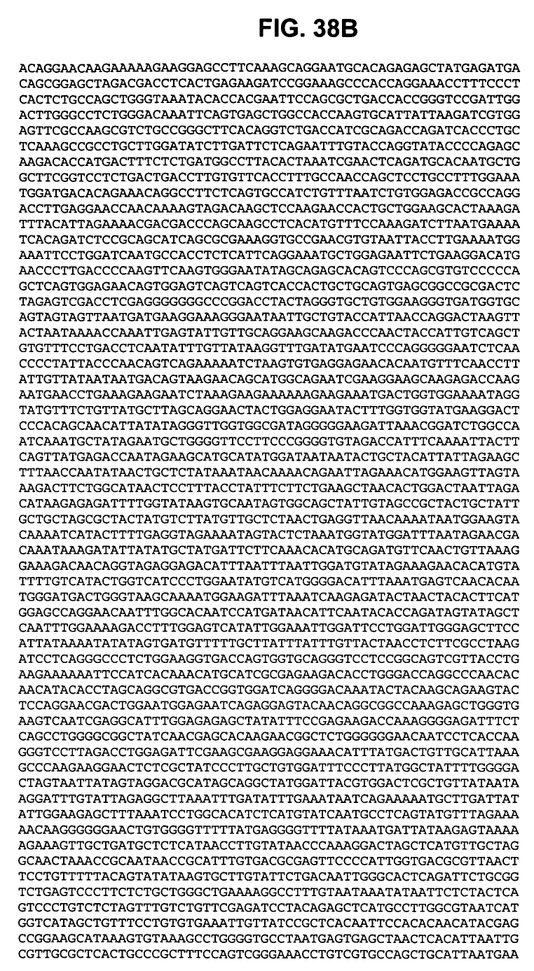

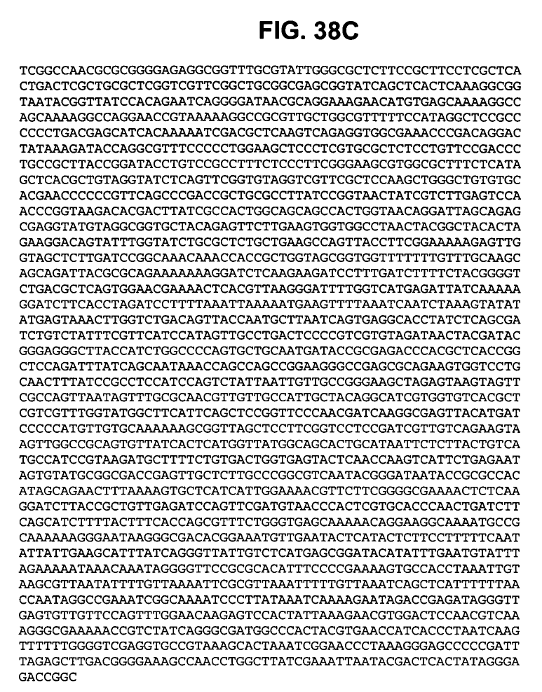

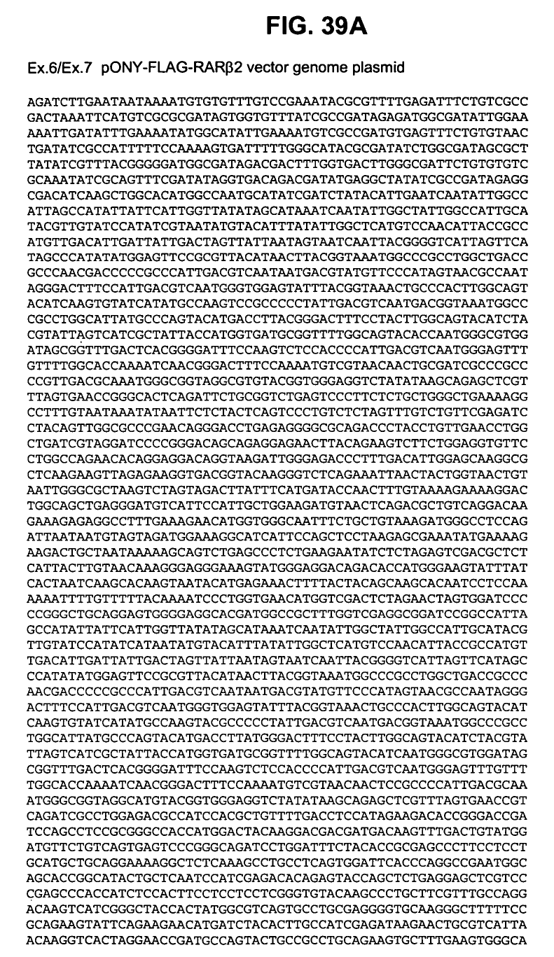

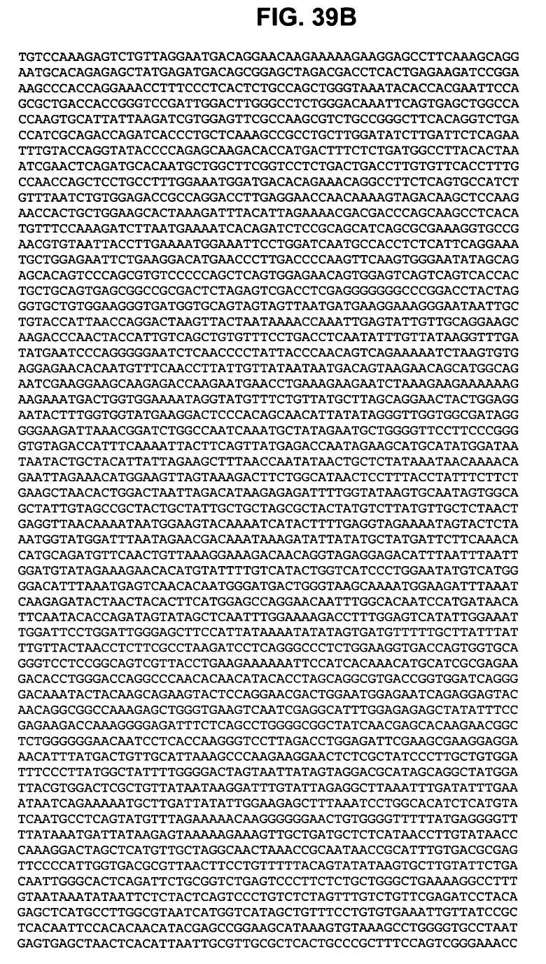

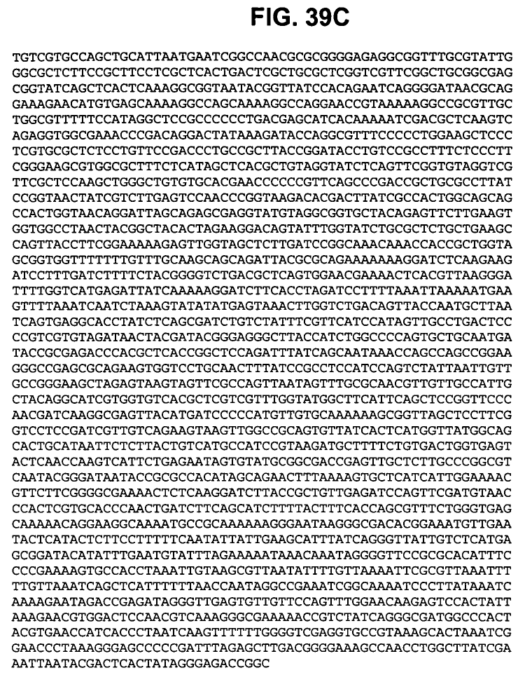

The resultant PCR products encoding RARfl2 or FLAG RAR-β2 (FIGS. 36 and 37) are digested with SacII and NotI and ligated into the EIAV vector genome, pONY8G 5′cPPT POS delCTS prepared for ligation by digestion with SacII and NotI. These enzymes cut the DNA on either side of the enhanced green fluorescent protein (eGFP) reporter gene. The resulting EIAV vector carrying the RARβ2 of FLAG RAR-β2 insert are termed pONY-RARβ2 and pONY-FLAG-RARβ2, respectively (FIGS. 38 and 39). Vector genome plasmid pONY8G 5′cPPT POS delCTS is a derivative of pONY8.0Z constructed as described in the following paragraphs.

Construction of pONY8G 5′cPPT POS del CTS

The presence of a sequence termed the central polypurine tract and central termination sequence (cPPT-see Stetor et al. Biochemistry. 1999 Mar. 23;38(12):3656-6) improves the efficiency of gene delivery to non-dividing cells (WO 99/55892). This cis-acting element is located in the polymerase coding region element and can be obtained as a functional element by using PCR amplification using any plasmid which contains the EIAV polymerase coding region (for example pONY3.1) as follows. The PCR product includes the central polypurine tract and the central termination sequence (CTS). The oligonucleotide primers used in the PCR reaction are:

EX7 EIAV cPPT POS:
CAGGTTATTCTAGAGTCGACGCTCTCATTACTTGTAAC

EX7 EIAV cPPT NEG:
CGAATGCGTTCTAGAGTCGACCATGTTCACCAGGGATTTTG

Recognition sequences for XbaI are shown in italic and use of this enzyme facilitates insertion of the PCR product into the pONY8G backbone.

Before insertion of the cPPT/CTS PCR product prepared as described above, the vector backbone is modified to remove the CTS which is already present due the presence of some EIAV pol sequences downstream of the reporter gene. This is achieved by subcloning the SalI to ScaI fragment encompassing the CTS and RRE region from pONY8.0Z into pSP72 (Genbank Acc.No.X65332), prepared for ligation by digestion with SalI and EcORV. The CTS region is then removed by digestion with KpnI and PpuMI, the overhanging ends ‘blunted’ by T4 DNA polymerase treatment and then the ends religated. The modified EIAV vector fragment is then excised using SalI and NheI and ligated into pONY8G prepared for ligation by digestion with the same enzymes. This new EIAV vector is termed pONY8G delCTS. pONY8G is derived from pONY8.0Z by exchange of the LacZ reporter gene for the enhanced green fluorescent protein (GFP) gene. This is done by transferring the SacII-KpnI fragment corresponding to the GFP gene and flanking sequences from pONY2.13GFP (WO 99/32646) into pONY8.0Z cut with the same enzymes.

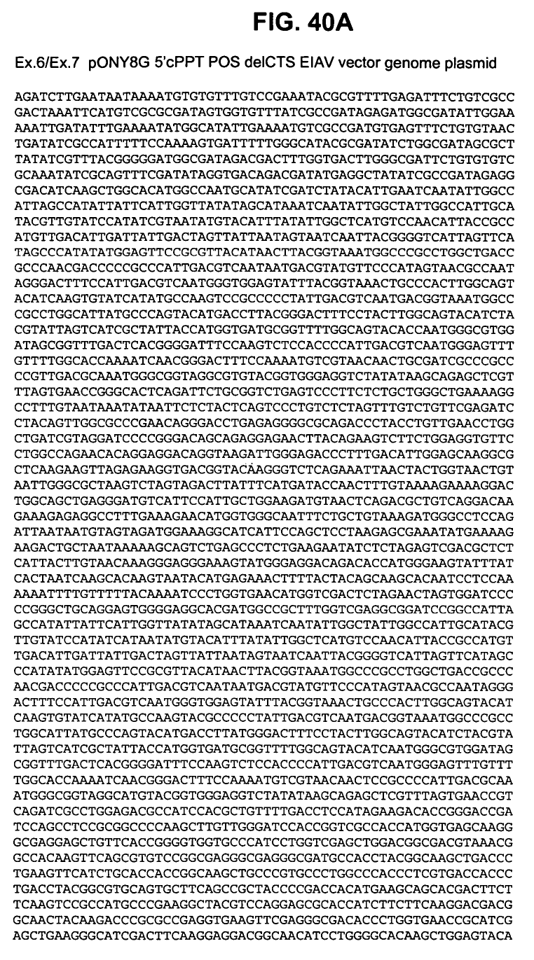

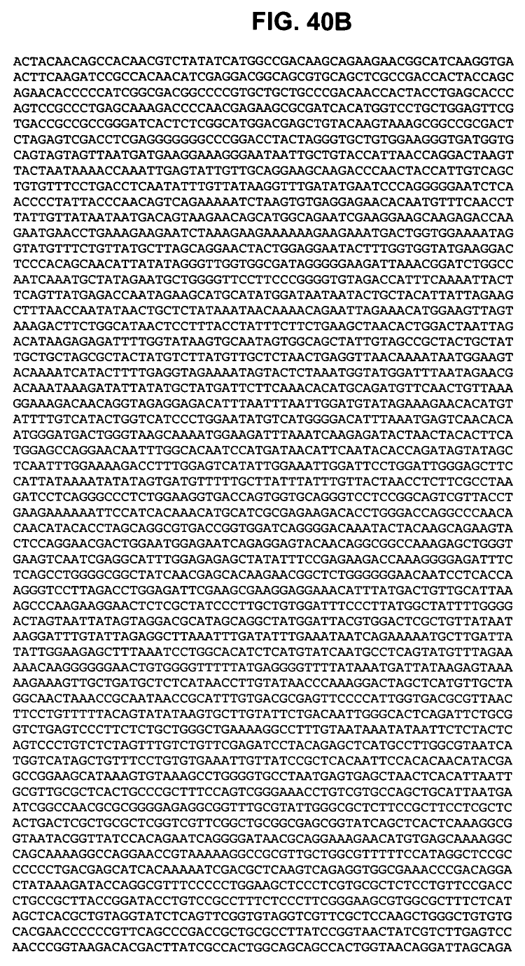

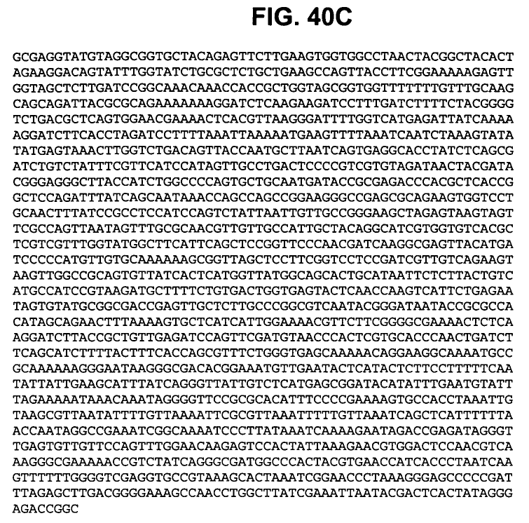

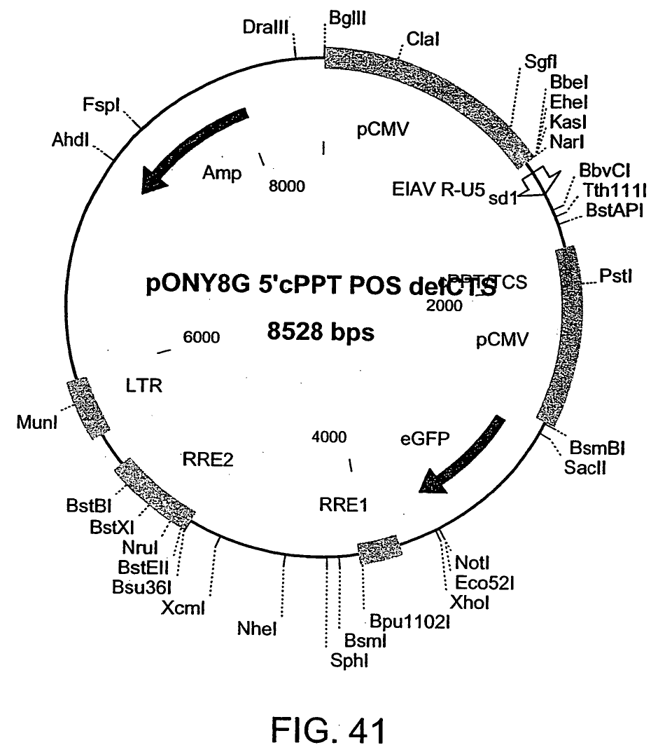

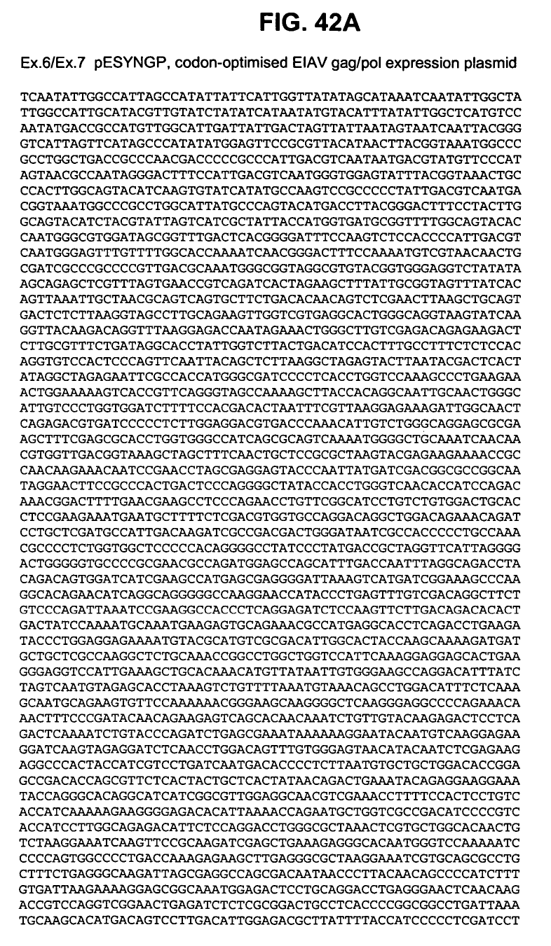

pONY8G delCTS has two XbaI sites which are located immediately upstream and downstream of the CMV-LacZ unit, respectively. The cPPT/CTS PCR product is digested with XbaI and then ligated into pONY8G delCTS prepared for ligation by partial digestion with either XbaI. Ligation into these sites results in plasmids with the cPPT/CTS element in either the positive or negative senses. A clone in which the cPPT/CTS is in the positive sense (functionally active) and located to the 5′-side of the intermal CMVpromoter was selected and termed pONY8G 5′cPPT POS delCTS (FIGS. 40 and 41).

Production of pONY-RARβ2 Vector Preparations

Vector preparations are made by transient co-transfection of HEK 293T, human embryonic kidney cells as described above (see Example 6) for pONY8.0Z, except that the vector genome plasmid was the pONY-RARβ2 vector genome plasmid. Vectors are made using either envelope expression plasmid pRV67 (which encodes VSV-G), pRabG or derivatives of pRabG (which encode Rabies virus G protein). However, expression plasmids encoding other proteins capable of pseudotyping EIAV may equally be used.

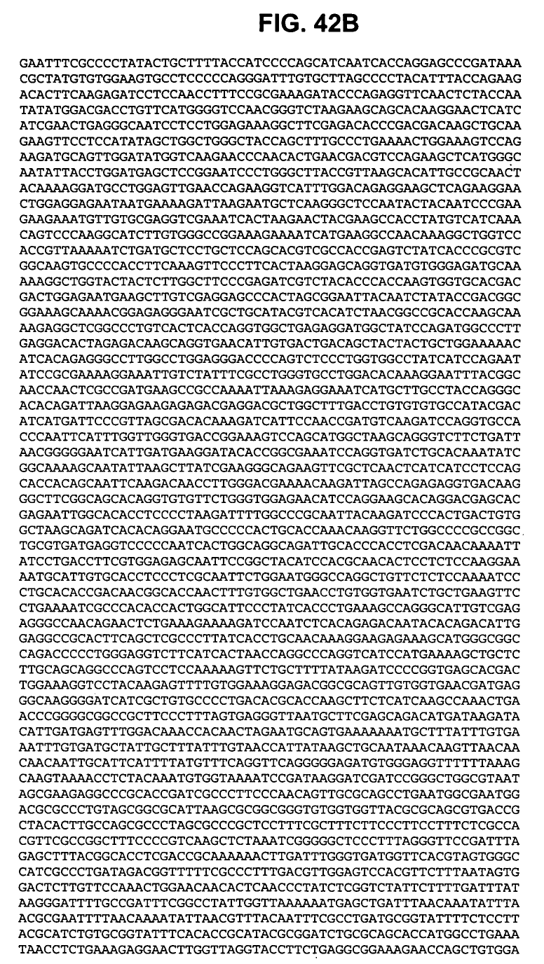

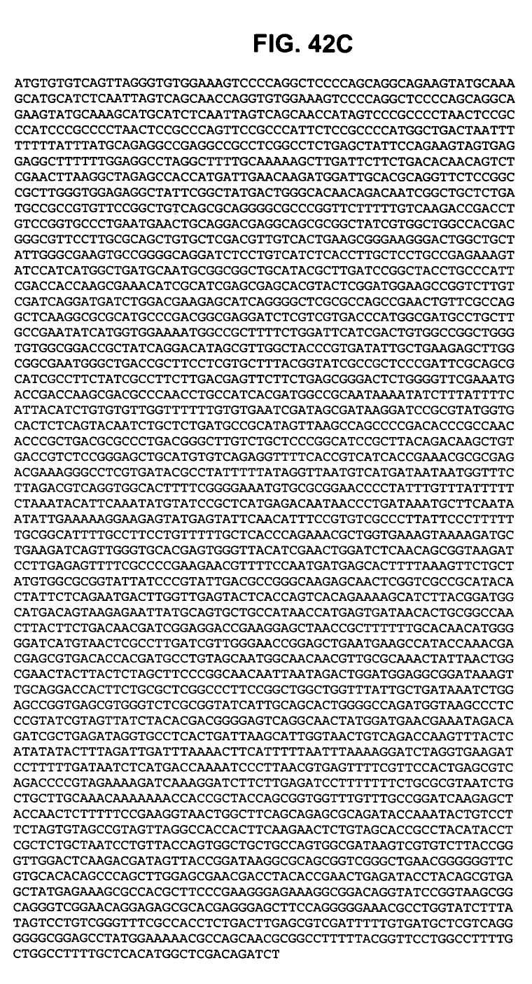

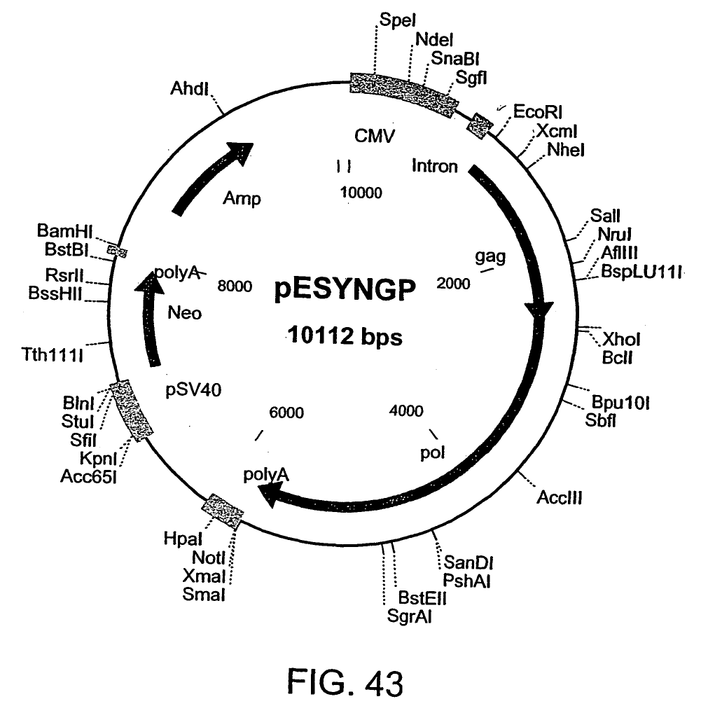

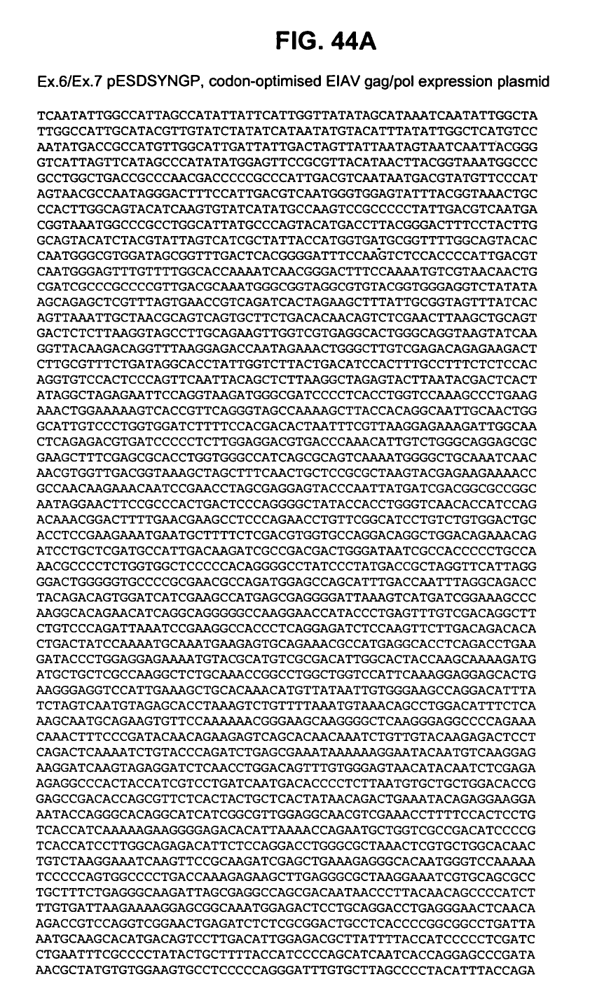

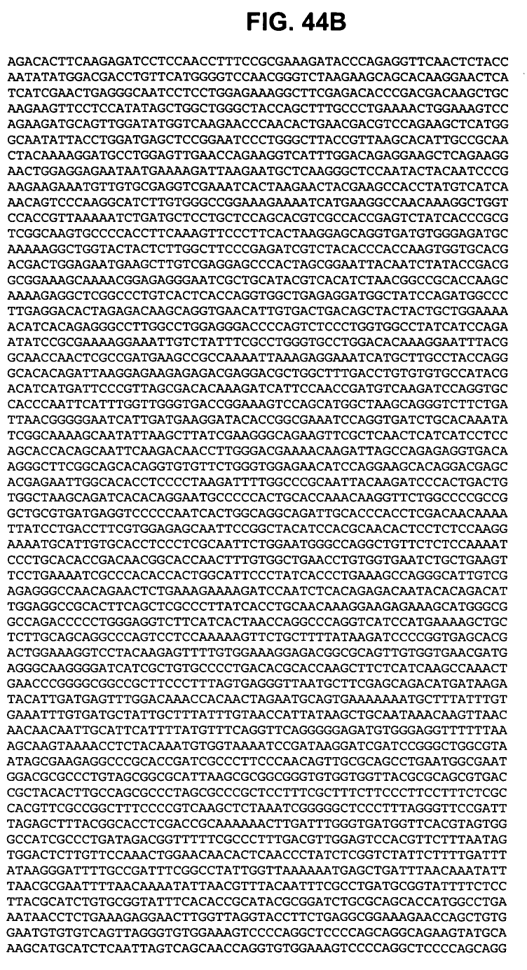

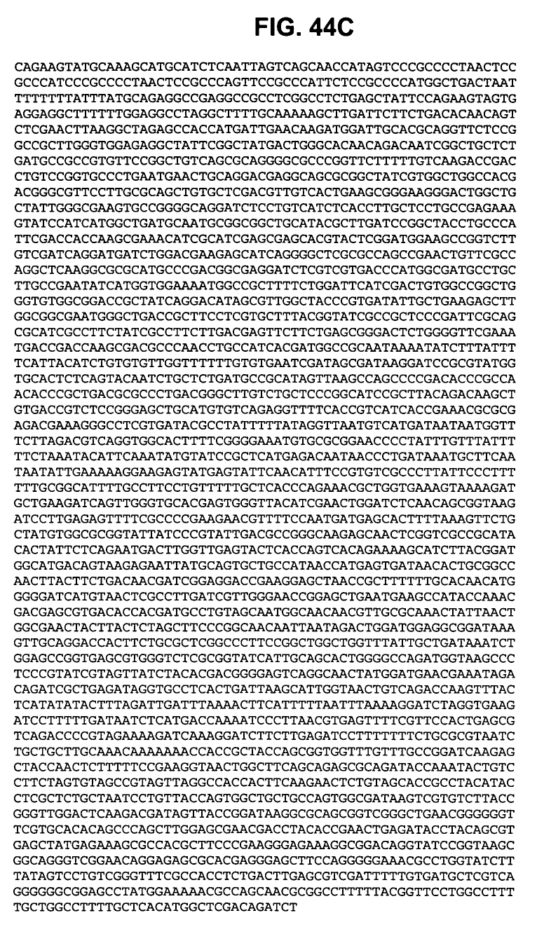

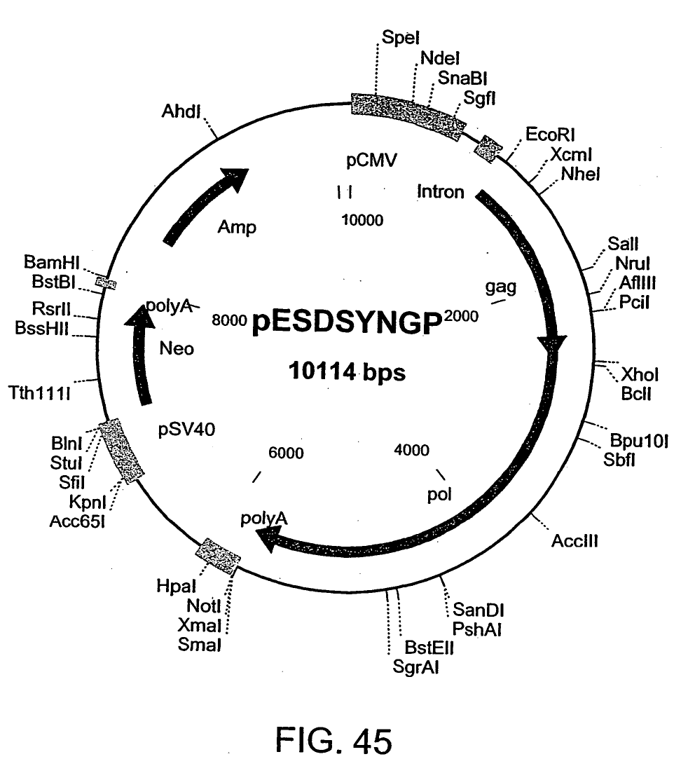

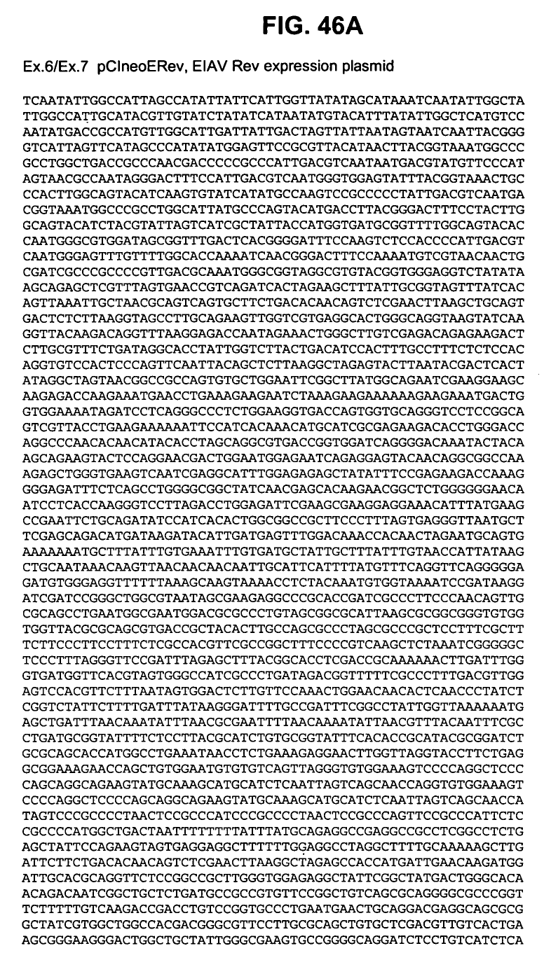

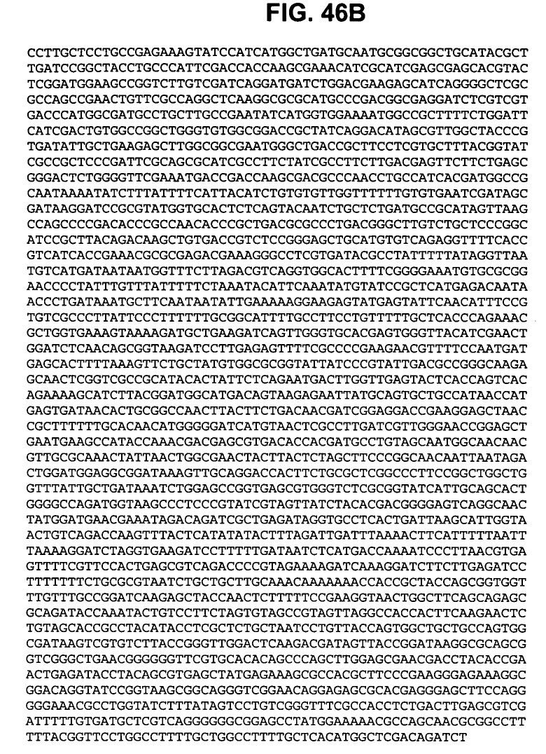

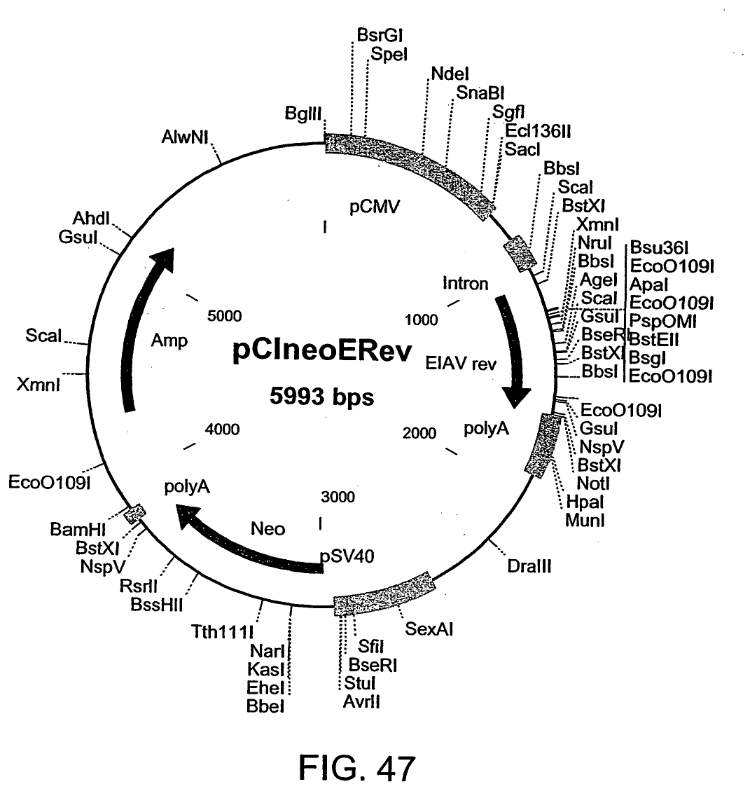

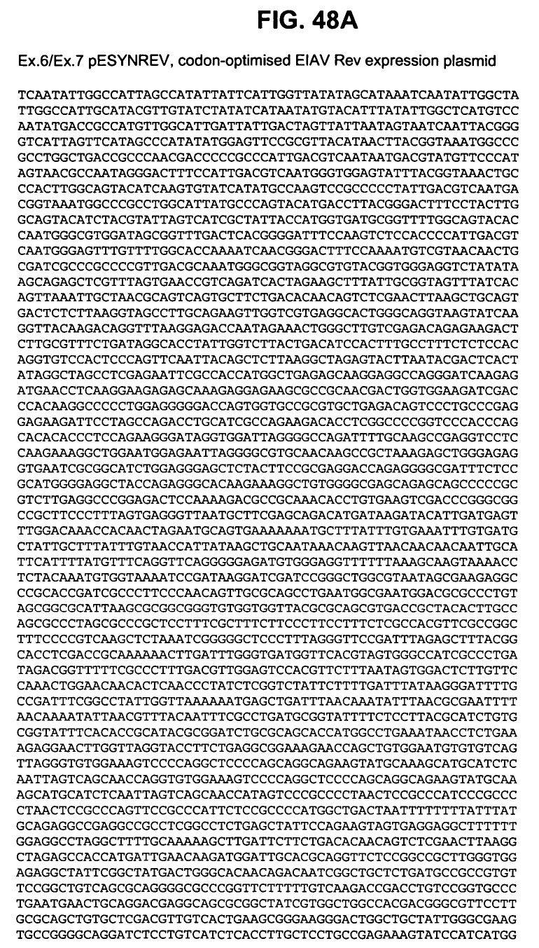

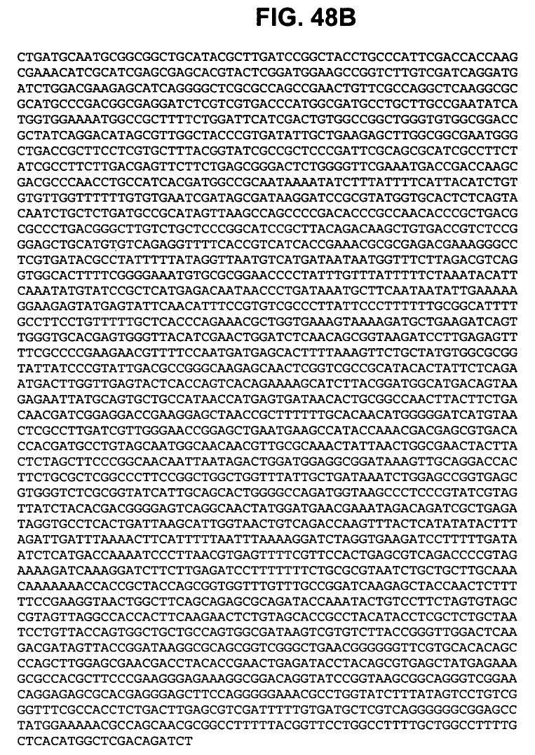

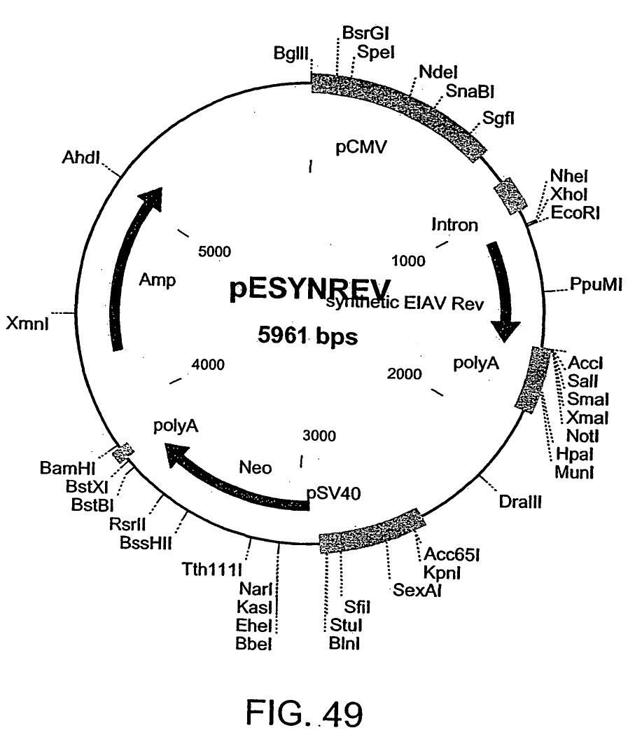

Vector may be made using Gag/Pol expression plasmids other than pONY3.1. For example, they can be made using the pESYNGP plasmid (FIGS. 42 and 43), in which the sequence of gag/pol gene is altered to optimise expression in human cells. This process is termed ‘codon-optimisation’ (Kotsopoulou et al., (2000) J. Virol. 74, 4839-4852 and GB0009760.2). pESYNGP is made by transferring a XbaI-NotI fragment from a plasmid containing a codon-optimised EIAV gag/pol ORF into pCIneo (Promega). The gene is synthesised by Operon Technologies Inc., Alameda, Calif. and supplied in a proprietary plasmid backbone, GeneOp. The complete fragment transferred includes sequences flanking the EIAV gag/pol ORF: tctagaGAATTCGCCACCATG-EIAV gag/pol-UGAACCCGGGgcggccgc. The ATG start and UGA stop codons are shown in bold and the recognition sequences for XbaI and NotI sites in lower case. Alternatively, pESDSYNGP (FIGS. 44 and 45) which has a splice donor in the leader can be used. pESDSYNGP was made from pESYNGP by exchange of the 306 bp EcORI-NheI fragment, which runs from just upstream of the start codon for gag/pol to approximately 300 base pairs inside the gag/pol ORF with a 308 bp EcORI-NheI fragment derived by digestion of a PCR product made using pESYNGP as template and using the following primers: EX7 SD FOR [GGCTAGAGAATTCCAGGTAAGATGGGCGATCCCCTCACCTGG] and EX7 SD REV [TTGGGTACTCCTCGCTAGGTTC]. This manipulation replaces the Kozak concensus sequence upstream of the ATG in pESYNGP with the splice donor found in EIAV. The sequence between the EcORI site and the ATG of gag/pol is thus CAGGTAAG. When transfections are carried out to make vector preparations using codon-optimised Gag/Pol expression plasmids higher yields of vector are obtained if a fourth plasmid encoding EIAV REV is also included in the transfection. Plasmids suitable for expression of EIAV Rev protein are pCIneoERev (WO 99/32646)(FIGS. 46 and 47) and pESYNREV (GB0009760.2) (FIGS. 48 and 49).

pESYNREV which is a pCIneo-based plasmid (Promega) which is made by introducing the EcoRI to SalI fragment from a synthetic EIAV REV plasmid, made by Operon Technologies Alameda, Calif., and contains a codon-optimised EIAV REV open reading frame flanked by EcoRI and SalI recognition sequences.

Transfections, harvesting and concentration of the vector particles are carried out as described above for pONY8.0Z.

Assessement of the Titre of pONY-RARβ2 Vector Preparations

The pONY-RARβ2 vector preparations lack β-galasctosidase or GFP markers which are commonly used to assess titre. However, titre of a vector preparation can be assessed, relative to a preparation of pONY8.0Z or pONY8G vector of known biological titre, by comparison of the levels of vector RNA incorporated into particles. This validity of this measurement method is dependent on equivalent efficiencies of vector entry, reverse transcription and integration for the vectors being compared. Similar methodology can be used to assess titre by direct measurement of integrated vector DNA in chromosomes of target cells. In this approach a direct measurement of biological titre is made.

A) Assessment of Titre Via Measurement of Packaged Vector RNA

The biological titre of the vector is related to the amount of vector RNA packaged in virions and can be assessed using quantitative RT-PCR using the ABI PRISM 7700 Sequence Detector. Any sequence within the vector genomic RNA can be used as a target however it should be unique to the vector component of the production system. A convenient target is the packaging signal. Vector RNA is isolated from the vector stocks using a Qiagen RNA isolation kit (Qiagen) and then DNAse treated using RNAse free DNAse (Ambion). Serial dilutions of the RNA are used as template in the RT-PCR reaction. Two TaqMan (Perkin-Elmer) reaction mixes are prepared, plus-RT and minus-RT, containing 1/10th volume of RNA template and the specific forward and reverse primers and probe. The minus-RT reaction is used to assess the efficiency of DNAse treatment. RT-PCR is carried out on an ABI PRISM 7700 Sequence Detector using conditions as follows: Hold, 48° C. for 30 min; hold, 95° C. for 10 min; forty cycles, 95° C. for 15sec and 60° C. for Imin. The data is analysed using the TaqMan software (Perkin-Elmer).

EX7 NEGATIVE SENSE PRIMER, FOR REVERSE
TRANSCRIPTASE STEP =
5′-accagtagttaatttctgagacccttgta
EX7 PLUS SENSE PRIMER =
5′ ATTGGGAGACCCTTTGACATT
EX7 PROBE =
5′ FAM-CACCTTCTCTAACTTCTTGAGCGCCTTGCT-(TAMRA)p3′

(This set of primers detects vector genome, but not wild type gag/pol.)

A) Assessment of Titre Via Measurement of Integrated Vector DNA

A similar approach to the above is used except that DNA is prepared from cells transduced with pONY-RARβ2 or pONY-FLAG-RARβ2, or the vector standard, using a Qiagen QIAamp DNA Mini Kit and the RT step is omitted.

Thus, it is demonstrated that vector particles for the delivery of RARβ2 may be produced according to the invention.

### Example 8

### Gene Transfer to Adult Nervous Tissue

The vectors of the present invention may be introduced into a subject by direct administration. In this example, it is demonstrated how nucleic acid expression constructs of the present invention can transduced into adult non-dividing neural cells using psudotyped EIAV-derived vectors as described above. It is further demonstrated that robust gene expression mediated by the vectors of the present invention is observed in vivo following gene transfer as disclosed herein.

Intraspinal Injection of EIAV Vectors

Lentiviral vector is used to facilitate direct in vivo gene transfer, and to express the reporter gene β-galactosidase in rodent spinal cord cells. A system comprising a stereotaxic frame and an automatic micropump allows the localised injection of viral stock solution into the rat spinal cord without inducing any significant damage (Azzouz et al., 2000 Hum Mol Genet. vol 9 pp803-11). In this Example, this is accomplished as follows:

Rats are anesthetized with an intraperitoneal injection of mixture solution of Hypnorm and Hypnovel (Wood et al., 1994 Gene Therapy vol 1 pp283-291). Animals are placed in a stereotax and their spinal cords are immobilized using a spinal adapter (Stoelting Co., IL, USA). EIAV vector is injected into the lumbar spinal cord following laminectomy.

To assess transduction efficiency of EIAV vectors into the spinal cord, 2 months old Albino rats are injected with 1 ul EIAVLacZ pseudotyped with VSV-G envelope (n=3) (6×10⁸ T.U./ml) at one site. Injections, controlled by an infusion pump (World Precision Instruments Inc., Sarasota, USA), are at 0.1 ul/min through a 10 ul Hamilton syringe fitted with a 33 gauge needle. Following injection, the needle is left in place for 5 minutes before being retrieved. Three weeks following virus injection, animals are perfused transcardially with 4% paraformaldehyde. The lumbar spinal cord is dissected out and histological analysis is performed.

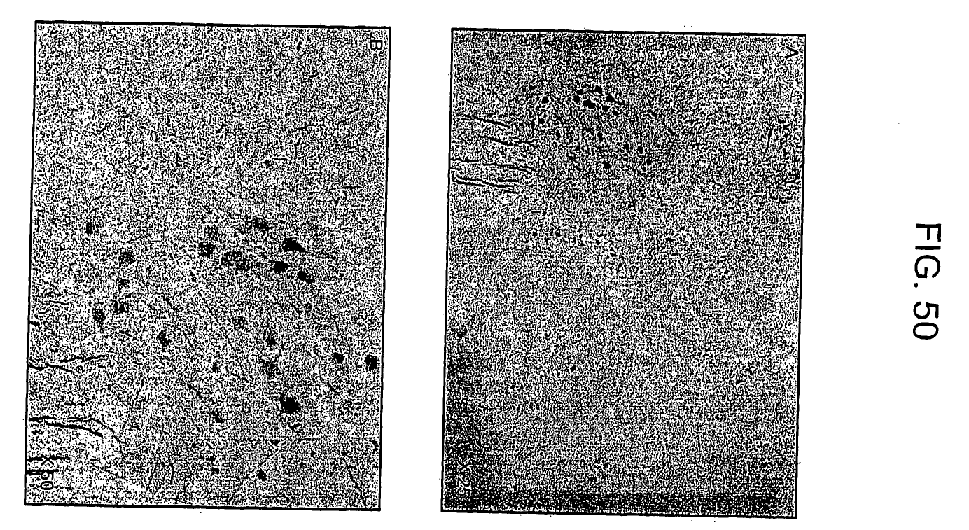

Intraspinal injection of the lentiviral vector is associated with only a mild degree of inflammation, with no significant cell damage. All subjects tolerated the surgery and vector injections with no detectable complications. Subjects continue to move normally in the cage post-injection, indicating the absence of functional deterioration following intraspinal injection of the viral vector. Both histochemistry (x-gal staining) and immunofluorescence reveal robust reporter gene expression within VSV-G injected spinal cord (FIG. 50).

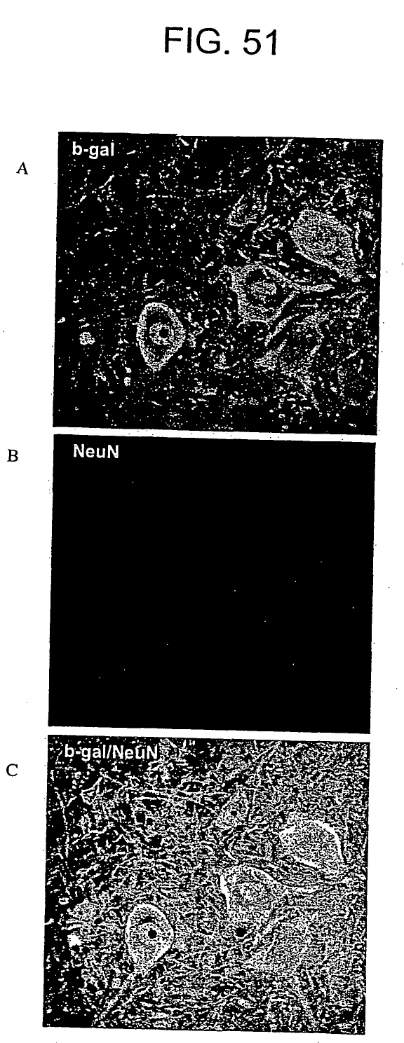

Transverse sections of the spinal cord reveal high transduction efficiency of the VSV-G pseudotyped vector. To demonstrate the phenotype of these cells, sections are double-labelled with antibodies to NeuN and to β-gal. At least about 90% of the transduced cells are double-labeled with NeuN in VSV-G pseudotyped vector injected sections (FIG. 51).

EIAV Injection into the Rat Lumbar DRG

The protocol described above is adapted for direct injection of EIAV based vector in the DRG. Briefly, DRG (levels L4/L5) are surgically exposed by dissecting the musculus multifidus and the musculus longissimus lumborum and by removing the processus accessorius and parts of the processus transversus. EIAV vector coding for the reporter gene β-gal (˜2-5×10⁹ TU/ml) is injected directly into the DRG. Rats receive 1 ul of the viral vector solution per ganglion. All injections are carried out using a stereotaxic frame and a Hamilton syringe with 34-gauge needle. The solution is slowly infused at the speed of approximately 0.1 ul/min. To confirm the transduction of sensory neurons by EIAV vector, histology and immunohistology using β-gal antibodies (Affiniti) are performed at 2, 4 and 8 weeks.

### Example 9

### Regenerative Properties of Retinoic Acid Receptor β2 (RARβ2) in Animal Models of Nerve Injury

The regenerative properties of RARβ2 were investigated in 2 animal models of nerve injury: the rhizotomy model (depicting peripheral nerve injury) and the corticospinal tract lesion model (mimicking spinal cord injury). Our anatomical studies revealed that EIAV vector delivery of RARβ2 improved the growth of injured dorsal root axons into the central region of the dorsal root entry zone (DREZ). By counting the number of regenerating fibres crossing into the DREZ 10 days after dorsal root crush, it was demonstrated that 32% of distally labelled fibres in the peripheral root entered the DREZ in RARβ2-transduced animals, compared to 10% in the control LacZ-transduced animals. In the corticospinal tract lesion model, RARβ2 also appeared to promote the regeneration of descending corticospinal tract fibres up to the lesion after a dorsal column crush injury to the cervical spinal cord. Based on these preliminary data supporting the regenerative capabilities of RARβ2 in in vivo models of nerve injury, longer term studies were subsequently initiated. These studies included longer survival times after injury, during which both treated and untreated animals were subjected to a variety of sensory and locomotor tasks. These behavioural tests served to determine if the anatomical observations of regeneration by RARβ2 could be recapitulated in functional correction of behaviour. Preliminary results indicated that treatment with RARβ2 led to correction of behavioural deficits that were elicited after nerve injury. These results further support that RARβ2 promotes regeneration of injured fibres leading to functional recovery.

Method

Viral Preparations

Viral preparations of SMART2.Z, SMART2.RARβ2 and SMART2.GDNF were prepared using standard protocol. The titres of the respective EIAV vectors, pseudotyped with ERAwt envelope, were 4×10⁸ T.U./ml, 8×10⁸ T.U./ml and 9×10⁸ T.U./ml (Quarterly report 03 Q1 Innurex LFW). The titres of the VSV-G pseudotyped vectors were 9×10⁸ T.U./ml, 7×10⁸ T.U./ml and 3×10⁹ T.U./ml respectively.

1) Cervical Rhizotomy Model

Viral vectors that were pseudotyped with rabies ERAwt envelope were injected unilaterally (4×2 μl) into the left side of the cervical spinal cord (C₄₋₈ level) 3 weeks before the corresponding roots on the same side of the animal were crushed. MW10 000 biotinylated dextran amine (BDA) tracer was injected into the corresponding dorsal root ganglions (DRG) at the time of the crush. Prior to injury and after rhizotomy, animals were tested for sensory and locomotor functions in several behavioural tasks such as staircase test, tape sensing and removal test, ladder crossing, beam crossing and footprint analysis (described below). Animals were sacrificed 4 weeks later for histological analysis. At the time of sacrifice, animals were anaesthetised with urethane and the left forelimb and hindlimb were dipped into a 56° C. waterbath to stimulate the primary sensory afferents. Thermal stimulation of the limbs will lead to an increase in c-Fos expression in the dorsal horn of the spinal cord, thus providing an additional test for functional regeneration of primary sensory afferents in treated animals.

2) Corticospinal Tract Lesion Model

A second set of experiments was performed. Vector injections were made into the motor cortex and left for 4 weeks. Bilateral injections of 6×1 μl were performed at a rate of 0.2 μl/min. A dorsal column crush was performed at the cervical (C4) level, after which the animals were monitored for sensorimotor functions using behavioural tests such as tape sensing and removal, ladder crossing, beam crossing and footprint analysis. 3 days after dorsal column crush, MW10 000 BDA was injected into the cortex to anterogradely label the corticospinal tract fibres. After a 6-week survival, the animals were sacrificed for histology.

3) Behavioural Assessment

Animals were subjected to a variety of sensory and locomotor tasks. In the staircase test (Montoya et al., 1991), the rat was placed on a platform on either side of which was a series of descending steps, each step containing a food pellet. The rat was restricted to reaching for the food pellet using either the left or right paw, and after 5 minutes, the number of food pellets retrieved on either side was scored. The tape removal test (adapted from Thallmair et al., 1998) provided separate scores for sensory and motor behaviour. Adhesive tape (1.5 cm×1 cm) was placed on the forepaw, the time taken to sense the presence of the tape (indicated by paw shake) was noted. For animals that sensed the tape, a removal time was also scored. In the locomotor tasks requiring sensorimotor integration, rats were trained to cross a narrow beam and a horizontal ladder suspended above the floor (Kunkel-Bagden et al., 1993). The number of forepaw foot slips were recorded (determined by a paw slipping off the beam or slipping below the plane of the ladder). Footprint analysis (Kunkel-Bagden et al., 1993) was determined by covering the forepaws with ink to record the walking patterns during continuous locomotion across a wooden runway, and stride lengths and widths were calculated.

Results

1) Cervical Rhizotomy Model (Bk 1316p171, Bk 1344p11, Bk 1344p71)

A total of 14 animals were used in this experiment. Animals were transduced with EIAV vectors encoding LacZ (n=4), RARβ2 (n=5) or GDNF (n=4). During the experiment, one RARβ2-transduced animal had to be sacrificed as the forelimb that was injured was severely mutilated. Behavioural data was recorded and the data is illustrated below.

Staircase Test

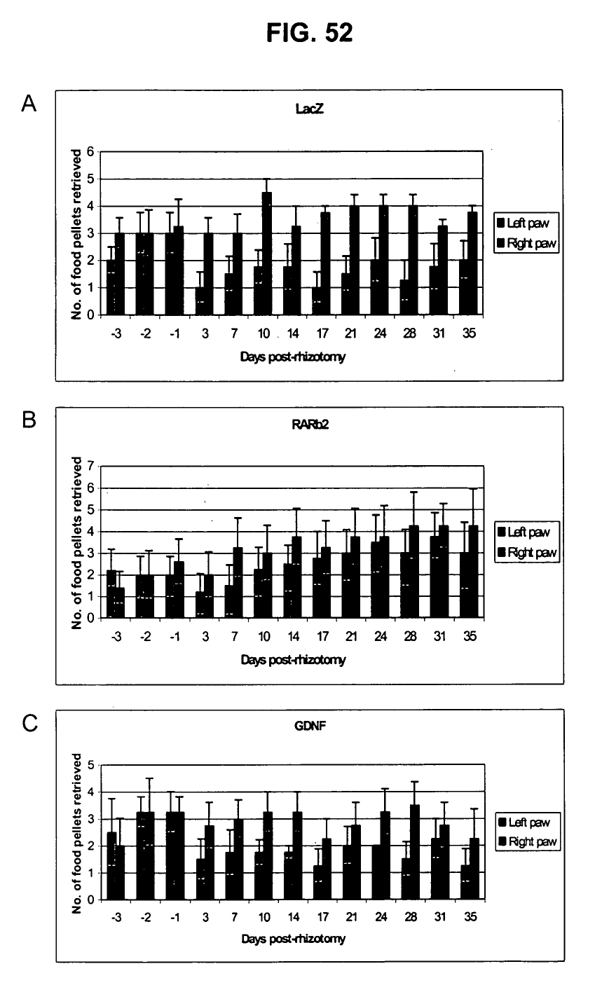

The number of food pellets that were retrieved with either the left or right paw were scored for the 3 groups of animals. Animals were pre-trained for 2 weeks before the time of dorsal root crush (day 0) and after injury, the animals were tested twice a week for 4 weeks. Prior to lesion there were no differences in the number of food pellets that were retrieved with the left or right paw in all 3 groups (FIG. 52). After crush injury was applied to the 4 dorsal roots innervating the left forelimb, the number of food pellets that were retrieved with the left paw was significantly lower in the 3 groups, compared with the right paw. This was sustained in the LacZ group throughout the testing period, up to 35 days after injury. In contrast, there was no significant preference for the left or right paw in the RARβ2 group even after injury to the left forelimb. This indicates that pre-treatment with RARβ2 allowed for recovery of the use of the left paw after injury, suggesting that RARβ2 either prevented the degeneration of injured axons at the earlier stages after injury or promoted the regeneration of injured fibres. In the GDNF group, there were no significant differences in left and right paw usage before injury; after injury the number of food pellets that were retrieved with the left paw decreased compared to the right paw however no significant preference for paw usage was detected. The preferential usage of the left paw over the right paw appeared to reach statistic difference at days 10, 14 and 28. The behavioural testing needs to be tested with more animals in order to obtain conclusive results with the GDNF group.

Tape Sensing and Removal Test

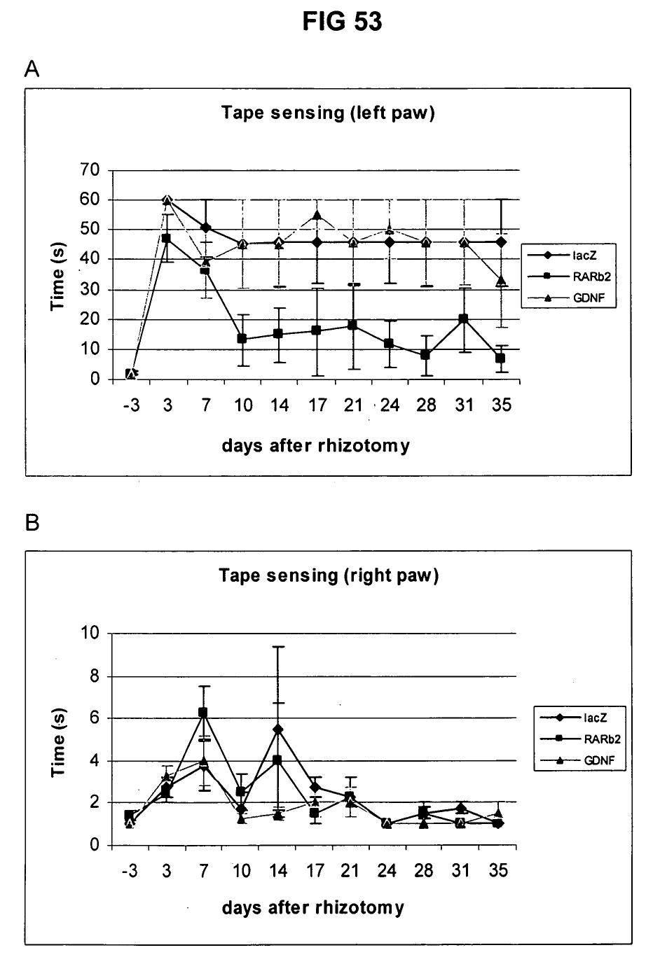

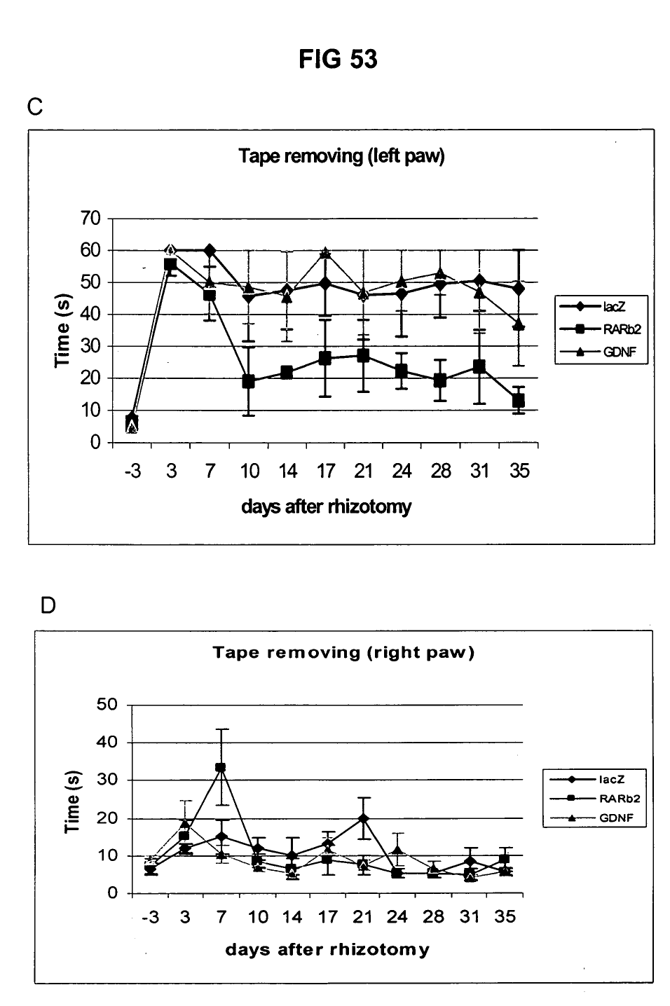

Animals were pre-tested before injury, during which the time taken to sense and remove the adhesive tape with either the left or right paw were below 5s and 10s respectively (FIG. 53). Immediately after injury, the time taken to sense the tape in the injured left forelimb increased significantly in all 3 groups (up to 60s), with no significant changes in the uninjured right forelimb. At 10 days after injury, there was a significant improvement in the RARβ2 group, where the time taken to sense and remove the tape with the left paw was significantly decreased (sense 13±8.4s; remove 19±11s) compared to the LacZ and GDNF groups (45.3±14.8s, 46±14s and 45.3±14.8s, 48.5±11.5s respectively). This was sustained throughout the testing period. No significant differences were detected with the right paw between the 3 groups throughout the study. Interestingly, the GDNF group did not show significant improvement in sensory function over time, as the time taken for the left paw to sense the tape was not significantly different from the control LacZ groups.

Ladder Crossing

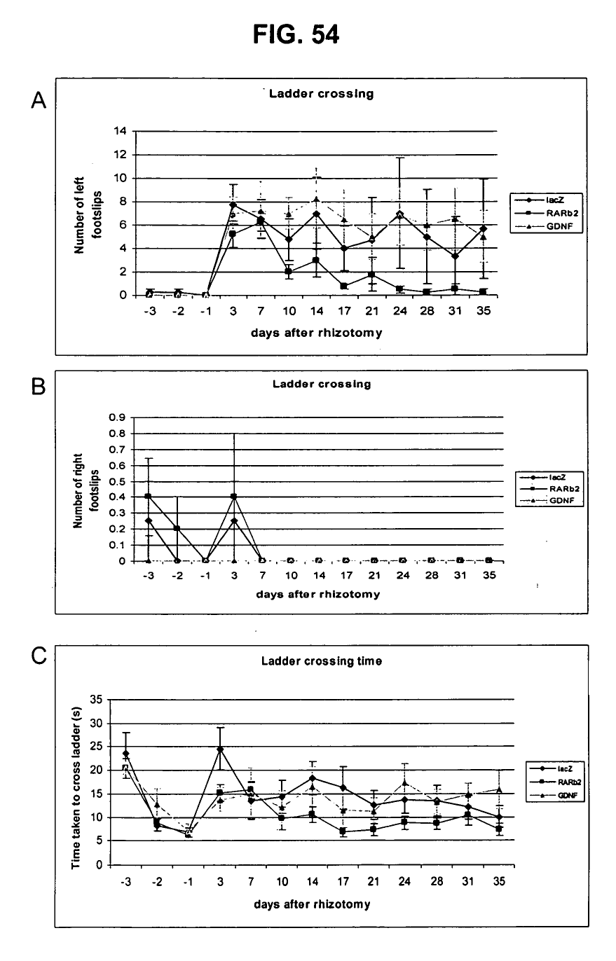

Animals were pre-tested prior to injury during which there no footslips made with the left forelimb. After injury the number of mistakes made with the left forelimb increased in all 3 groups. At day 10, the number of footslips decreased significantly in the RARβ2 group (2±0.58 left footslips) compared to the LacZ and GDNF groups (4.75±1.8 and 7.3±1.4 left footslips respectively; FIG. 54). This difference between the RARβ2 and LacZ groups was sustained up till 35 days. No significant increase in mistakes made by the right control forelimb were detected in the 3 groups throughout the study. Furthermore, the time taken to cross the ladder after injury was lower in the RARβ2 animals up till 28 days. This suggests that RARβ2 improved the locomotor deficits that were elicited by the dorsal root injury in this test.

Beam Crossing

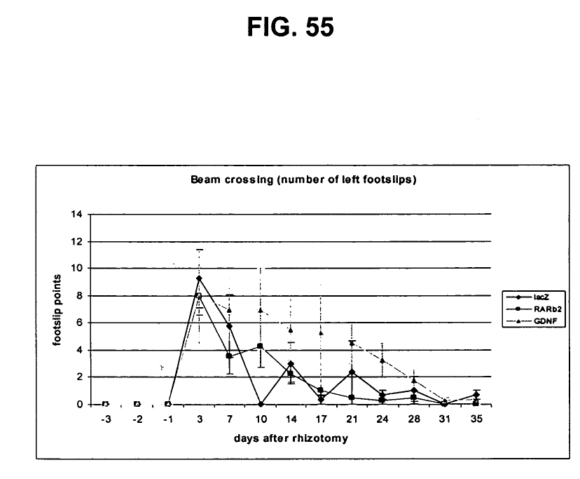

The number of footslips made by the left forelimb during beam crossing was recorded. After nerve injury, there were no significant differences in the number of mistakes made by the left forelimb between the 3 groups (FIG. 55); neither were there differences in the time taken to cross the beam. Over time, animals in all 3 groups made fewer mistakes during this task, suggesting that the animals may have learnt to cross the beam using 3 limbs.

Footprint Analysis

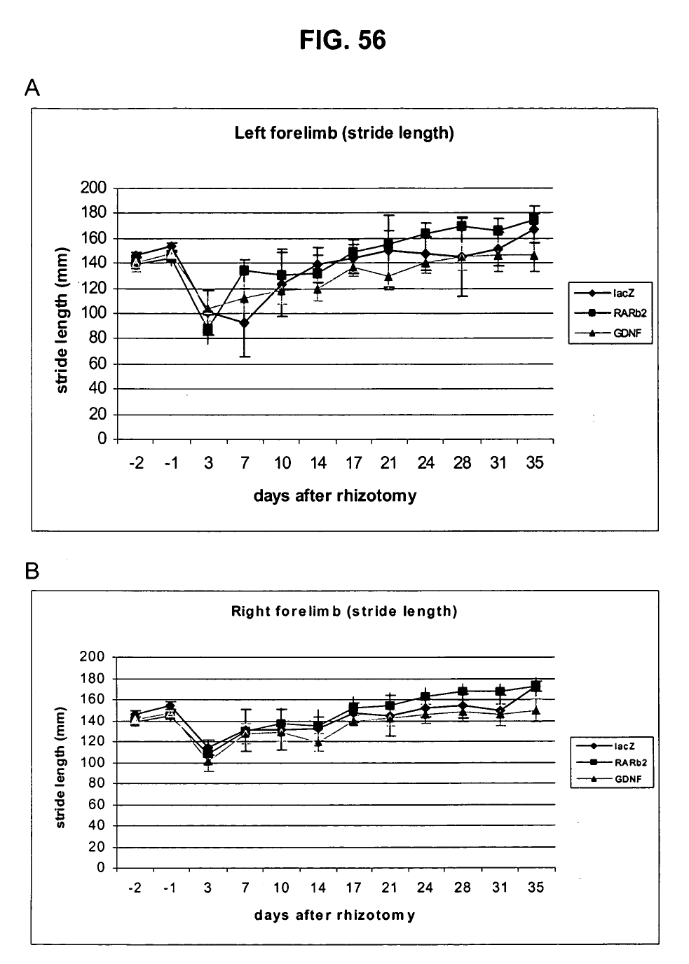

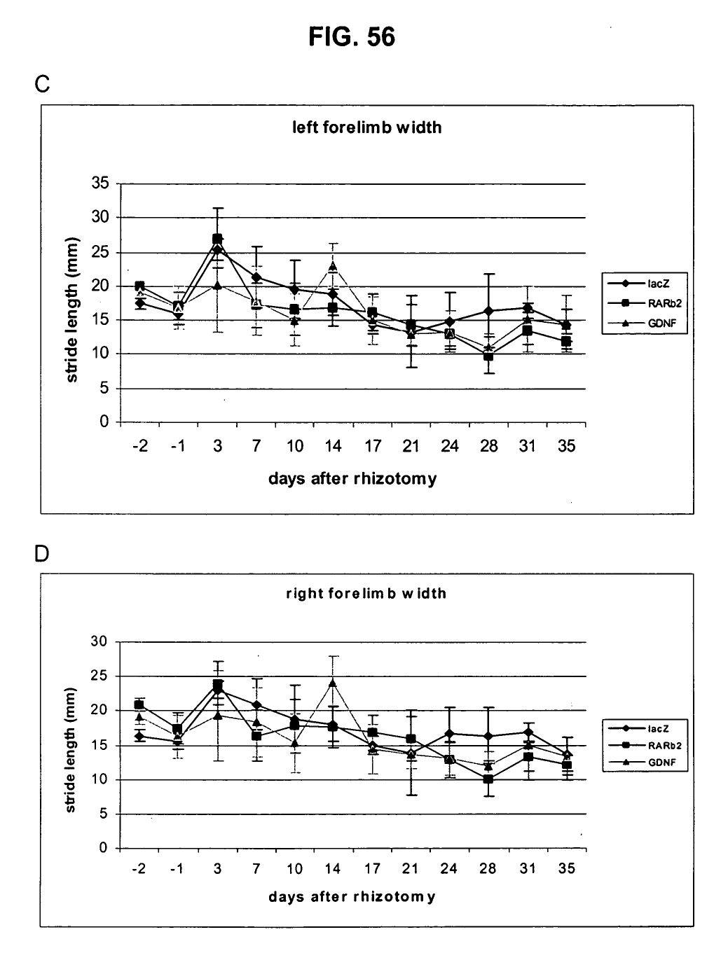

The stride lengths and limb widths of the left and right forelimb were analysed before and after injury. There were no significant differences between the 3 groups in either the left or right forelimbs, although in all groups the stride length in both limbs decreased immediately after injury (FIG. 56). This suggests that RARβ2 treatment did not affect this aspect of the locomotor behaviour.

2) Corticospinal Tract Lesion (CST) Model (Bk1344p96)

14 animals were used in this experiment. 4 rats were transduced with EIAV vectors expressing LacZ or GDNF and 5 rats were transduced with vectors expressing RARβ2; all vectors were pseudotyped with VSV-G envelope. During the study 2 GDNF animals died during operative procedures. Behavioural data was collected 1 week before and after spinal cord injury, and animals were sacrificed 6 weeks post-injury. During behavioural testing, an additional group of sham-operated rats were included as controls to illustrate the locomotor deficits that were induced by spinal cord injury.

Tape Sensing and Removal Test

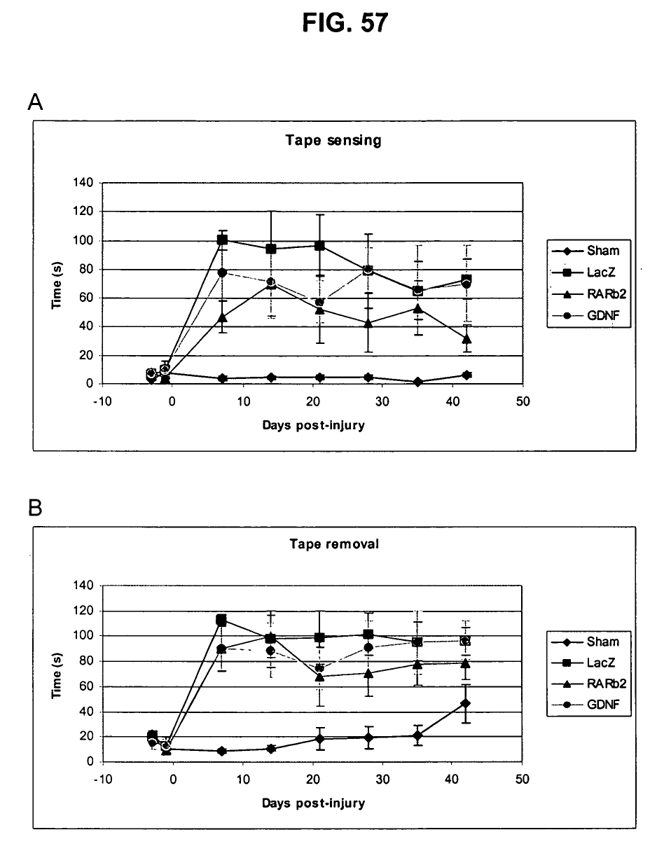

Right and left forepaw scores were averaged as the induced injury was bilateral and there were no significant differences between the left and right forelimb deficits. Prior to lesion the time taken for animals in all 3 groups to sense the adhesive tape was less than 10s; this time increased dramatically after spinal cord injury (FIG. 57). However the RARβ2 rats took a shorter time to sense the tape compared to the LacZ animals (46.7±11.3s and 100.3±6.9s respectively; sham animals 4±1.4s). This trend persisted throughout the testing period and at day 42 the time taken for tape sensing was also significantly lower in the RARβ2 group compared to the control LacZ group (31.5±9.4s and 73.1±1.4s). A similar trend was observed in the tape removal test, although the improvement induced by RARβ2 was less pronounced and was not significantly different from the control LacZ group. There were no significant differences between the GDNF and the LacZ groups.

Beam Crossing

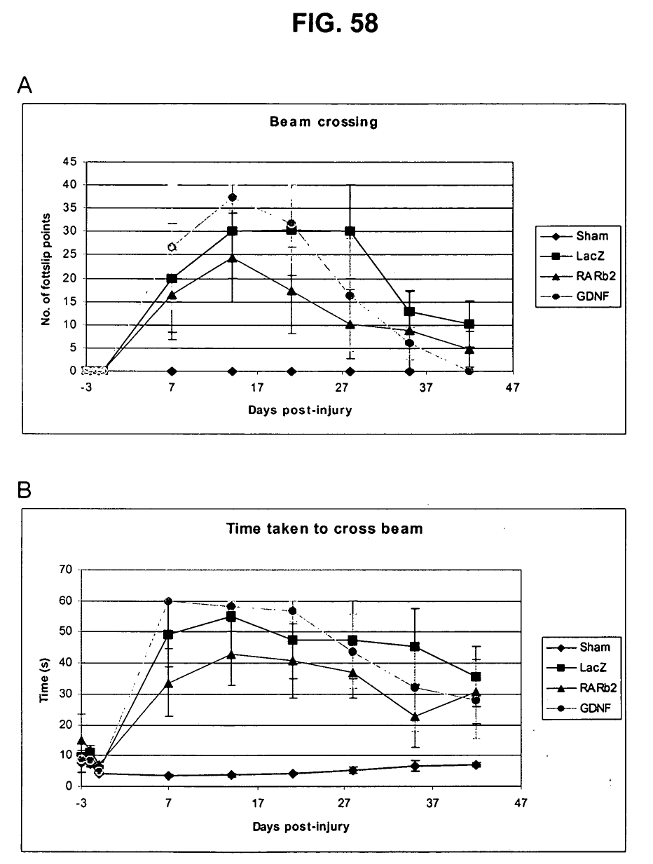

Animals in all 3 groups made a low number of mistakes during beam crossing and took a short time (less than 20s) to cross the beam before injury (FIG. 58). After injury lesioned animals were severely impaired in this locomotor task, however the RARβ2 animals made fewer footslips during beam crossing and took a shorter time to cross the beam indicating that RARβ2 treatment promoted partial recovery of function. There was also a modest recovery of function in the GDNF group.

Ladder Crossing

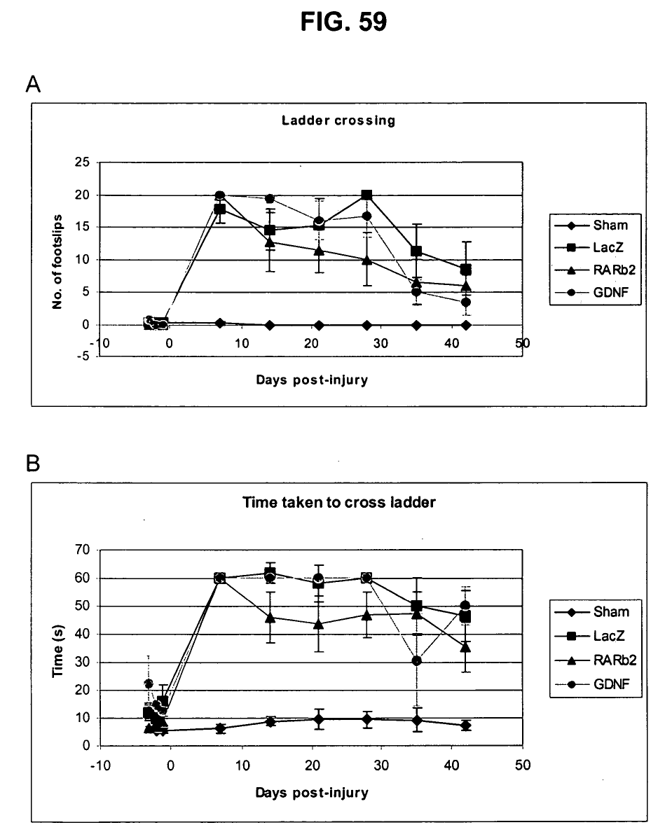

Lesioned animals were severely impaired in their ability to cross the horizontal ladder without making footslips and took up to 60s to cross the ladder (FIG. 59). At day 21 there was a small recovery of function in the RARβ2 group compared to the control LacZ group however this was not significant (11.4±3.5 and 15.3±4.1 footslip points respectively). No significant improvement was observed in the GDNF group.

Footprint Analysis

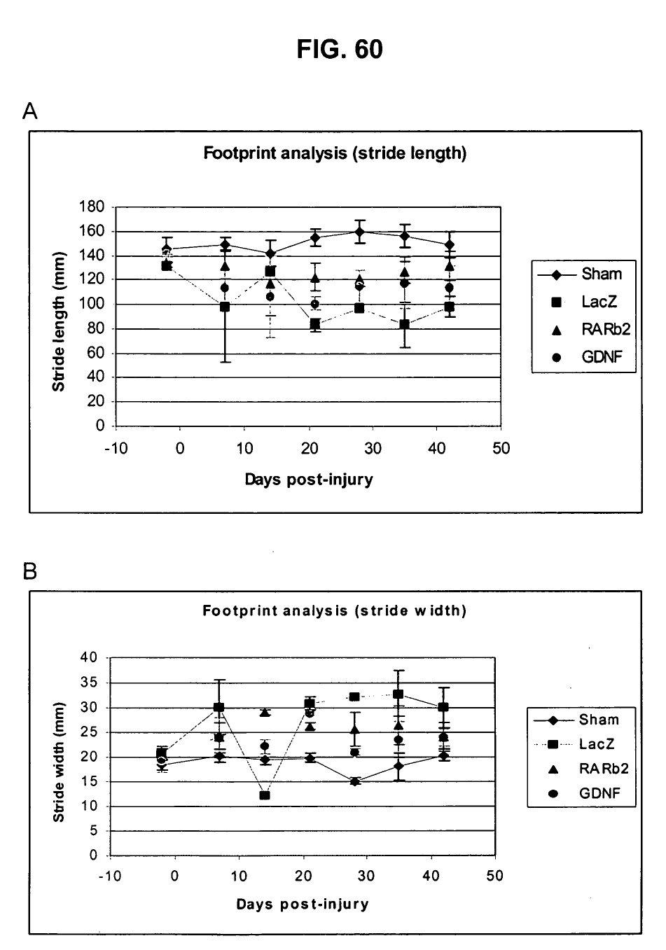

Walking patterns were assessed by analysing footprint spacing. Lesioned LacZ rats took shorter and wider strides (98.2+46.3 mm and 29.9+5.5 mm), compared to sham-operated rats (148.8±6.1 mm and 20.3±1.4mrnm; FIG. 60). Pre-treatment with RAR#2 resulted in longer and narrower strides (131.4±11.2 mm and 23.9±3.0 mm), although the walking patterns were not restored to that of sham-operated rats. Similarly, GDNF animals demonstrated an improvement in walking patterns (stride length and width 100±5.2 mm and 28.8±0.3 mm) at day 21, compared to LacZ rats (82.9±7.2 mm and 30.8±1.3 mm).

2. Discussion

1) Cervical Rhizotomy Model

Results from the behavioural assessment suggested that pre-treatment with RARβ2 promoted recovery of sensory and locomotor function in impaired rats in this model. This was illustrated by the improvement in sensory tasks such as tape sensing test, in locomotor tasks such as ladder crossing, and in integrated sensorimotor tasks such as the staircase test. No functional correction could be detected in the beam crossing test as all the animals demonstrated similar improvement in task performance over time. The animals may have learned to cross the beam using the 3 fully functional limbs without making mistakes; this could be achieved by dragging the injured left forelimb along the beam during crossing. As the functional deficit that was induced by the injury was not maintained in the control animals (LacZ) over the testing period, it was difficult to assess any positive effect RARβ2 or GDNF might have had. Similarly, footprint analysis did not demonstrate any significant differences between treated and untreated groups. Again, rhizotomy did not induce a large change in walking patterns in the control LacZ animals, and hence there was not a large enough deficit to correct. It is thus important to use a range of behaviour tests to determine which test is suitable as an assessment of functional outcome after treatment.

The behavioural correction effects observed with RARβ2 will be used to correlate to anatomical evidence of axonal ingrowth, past the DREZ and into laminae I/II in the grey matter of the spinal cord, where regenerating fibres can form functional connections to synapsing interneurones. Therefore the tissue from these animals are currently being analysed using immunohistochemistry. An important test of functional regeneration is the observation of c-Fos immunoreactivity in neurones located within the dorsal horn of the spinal cord after thermal stimulation in the forelimb, as these neurones receive sensory synaptic input from the peripheral limb.

There was no significant correction in the behavioural deficits in most of the tasks after GDNF treatment. This may be due to the poor quality of EIAV.GDNF vectors that was used in the experiment. Post-hoc analysis of the GDNF batch of vectors indicated that the integration titre of the vectors were at least 10-fold lower than the RNA titres, suggesting that the particular vector batch may not have adequate integration capacities. This will have a severe impact on GDNF expression in the transduced tissue and may therefore explain the lack of behavioural improvement in the GDNF group.

2) Corticospinal Tract Lesion Model

Sensory and locomotor tasks such as the tape sensing and removal test, beam crossing test and analysis of walking patterns demonstrated that RARβ2 promoted a modest but significant recovery of function in lesioned rats. The GDNF-treated animals also showed a modest improvement in behavioural tests in this study. The ladder crossing test gave less conclusive results in this model. This may be due to the fact that this particular task was a huge challenge to the rats as the rungs were spaced relatively far apart. To improve the significance of the results obtained in this study, it may be necessary to repeat the experiment to increase the sample size in order to reduce the variance in the deficits induced by injury. Again, the behavioural data will be used to correlate with anatomical evidence of axonal regeneration.

In this model, vectors were delivered to the sensorimotor cortex where the descending fibres of the corticospinal tract originate. The large distance between the site of delivery and the site of injury (cervical spinal cord) may reduce the effectiveness with which the vectors may promote regeneration. For example, if the regenerative action of RARβ2 was to occur via reduction of inflammation at the site of injury, delivery of RARβ2 to the sensorimotor cortex may reduce the efficacy of the vector. Furthermore, multiple injections had to be performed to transduce a large area occupied by the cortex and not all corticospinal neurones may have been transduced with 6 injections. Another potential site of delivery is at the site of injury—the cervical spinal cord.

3) Mechanism of Action of RARβ2

The mechanism by which RARβ2 may promote regeneration of nerve fibres is being investigated. RNA derived from tissue samples from rat spinal cord transduced with either LacZ, RARβ2 or GDNF were analysed on Affymetrix Neurobiology chips to determine if RARβ2 expression mediated gene changes in the spinal cord. The number of genes affected by RARβ2 or GDNF, relative to LacZ, are illustrated in the following table:

| | Upregulated ≧ 2 fold | Downregulated ≧ 2 fold |
|---|---|---|
| RARβ2 | 77 | 62 |
| GDNF | 12 | 17 |

Examples of genes that were affected by RARβ2 are given below:

| | Gene | |
|---|---|---|
| | change by | |
| Gene | RARβ2 | Comments |
| Glutamate receptors 1,3 | ↑2.6-3.8 | Involved in multiple sclerosis |
| α-synuclein | ↑2-2.2 | Mutant synuclein is involved in |
| | | Parkinson's disease |
| Zinc finger protein 179 | ↑4.1-11 | Transcription factor involved in |
| | | injury |
| Purinergic receptor P₂X₄ | ↓2.3-3.4 | P₂X₄ is induced in microglia in |
| | | neuropathic pain |
| Small inducible cytokine | ↓125-271 | Chemokine |
| A5 | | |
| β-transforming growth | ↓2.6-6.9 | Involved in scarring and wound |
| factor and receptor | | healing |
| neuropilin | ↓2.6 | SM has in situ probes to this |
| | | protein |

The gene changes caused by RARβ2 and GDNF could be classed into groups such as receptors, ion channels, synaptic transmission and structural proteins. More significantly a large number of genes that were downregulated by RARβ2 were caspases, receptors, cytokines and chemokines. This may be of significance in nerve regeneration as these inflammatory mediators are often upregulated in spinal cord injury and the regenerative action of RARβ2 may be mediated through downregulation of inflammatory activities in the spinal cord. The information that is derived from these microarray experiments will be useful in elucidating the mechanisms by which RARβ2 potentiates neurite regeneration in nerve injury. It will be useful to investigate the changes in protein expression of these inflammatory markers in the above models. Furthermore this information will be useful in planning further experiments to maximise the regenerative potential of RARβ2 in the above models.

### Example 10

### Regenerative Properties of Retinoic Acid Receptor β2 (RARβ2) in Animal Models of Nerve Injury

Preliminary results of Example 9 indicated that the EIAV vector expressing RARβ2 (Innurex™) improved the growth of injured dorsal root axons into the dorsal root entry zone (DREZ). This axonal regeneration was accompanied by an improvement in task performance in sensorimotor tasks such as the paw reaching, tape sensing/removal and ladder crossing tests, compared to the control (LacZ-transduced) group. Together these results support that RARβ2 promotes regeneration of injured sensory fibres leading to functional recovery.

Results from immunohistochemistry studies performed on tissue that was harvested from the above experiment confirmed that RARβ2 established functional connectivity in the spinal cord. Furthermore an additional experiment was initiated in order to increase the sample size and add statistical value to the behaviour data. In this latter experiment animals were treated with EIAV.RARβ2 or EIAV.LacZ after which the dorsal roots in each animal were crushed. Performance at various sensorimotor tasks were again assessed.

The RARβ2 that is encoded by the current Innurex™ vector is of mouse origin and therefore may not be optimal for therapeutic use in humans. In order to advance on the preclinical development of Innurex™, steps were undertaken to codon-optimise the human RARβ2 sequence. The newly synthesised human RARβ2 sequence was cloned into the pONY8.7 and pONY8.95 series of vectors and are currently being tested in vitro. The new RARβ2 sequence will also be cloned into the clinical vectors, after which they will be tested rigorously in the above model so as to obtain valuable preclinical data for bringing Innurex™ closer to the clinic.

Method

Immunohistochemistry on Harvested Tissue

At the termination of the study, animals were overdosed with anaesthesia and transcardially perfused with 0.9% saline followed by 4% paraformaldehyde. The dissected cervical spinal cord with the attached dorsal roots was post-fixed with 4% paraformaldehyde for 2 hours and cryoprotected in 20% sucrose. 20 μm sections were used for immunohistochemistry to detect the tracer biotinylated dextran amine (BDA) using Extravidin-FITC (Sigma), calcitonin gene related peptide (CGRP; Sigma) and pERK (Sigma).

Viral Preparations

Viral preparations of SMART2.Z and SMART2.RARβ2 were prepared using standard protocol. The titres of the respective EIAV vectors, pseudotyped with ERAwt envelope, were 5.9×10⁹ T.U./ml (biological titre) and 5.6×10⁸ T.U./ml (RNA titre; 8.4×10⁷ integrating copies/ml).

Cervical Rhizotomy Model

Viral vectors that were pseudotyped with rabies ERAwt envelope were injected unilaterally (4×2 μl) into the left side of the cervical spinal cord (C₄₋₈ level) 3 weeks before the corresponding roots on the same side of the animal were crushed. Prior to injury, animals were trained in various locomotor tasks such as paw reaching, tape sensing/removal, ladder crossing, beam crossing, grid crossing and footprint analysis (described in Quarterly report 03 Q3 Innurex LFW) and baseline measurements were taken. Following rhizotomy (4 corresponding roots C₄-C₈) the animals were again tested for sensorimotor functions.

Cloning of Codon Optimised RARβ2

The human RARβ2 sequence was submitted to GeneART (Germany) for codon optimisation. Codon usage was adapted to the codon bias of highly expressed mammalian genes in accordance to the codon usage table by Haas et al. (1996) thereby increasing translational efficiency. During optimisation, a Kozak sequence was introduced upstream of the starting ATG to increase translational initiation and negative cis-acting sites were removed. The codon optimised RARβ2 (CO—RARβ2) was excised out of pPCR-Script Amp with XhoI and KpnI and ligated into the pONY8.7NC and pONY8.95 vectors using the same enzymes. The resultant positive clones were identified and used to make unconcentrated EIAV preparations using standard protocol.

Results

1) Cervical Rhizotomy Model

Tissue from the previous rhizotomy study was utilised for histological analysis.

Immunohistochemistry was performed in order to detect axonal regeneration of injured sensory axons into the DREZ.

BDA Labelling

In the previous experiment injured sensory axons were labelled with the anterograde tracer biotinylated dextran amine (BDA; MW10000) at the time of the rhizotomy and were left for 5 weeks before termination. Immunohistochemistry was performed to detect the BDA in regenerating axons, however this was unsuccessful in all samples. The failure to detect the BDA in the dorsal roots could be due to several reasons. One, the quality of the BDA at the time of injection was questionable; two, the injection techniques were not satisfactory; or three, the rate of anterograde transport of the BDA. It is unlikely that the lack of BDA labelling in the samples was due to the first two reasons as the same batch of BDA has been utilised in other experiments with success. Furthermore at least 2 different operators performed the BDA injections and it is unlikely that all the labelling injections failed. It is more probably that the BDA was injected too early in the experiment (5 weeks) and that most of the BDA was anterogradely transported down the sensory axons into the spinal cord, leaving very little BDA in the dorsal roots. It has been established that the MW10000 BDA is capable of travelling at least 500 mm in 4 weeks (from the sensory cortex down the spinal cord) in corticospinal tract labelling studies. In contrast the distance between the DRG and the spinal cord is less than 5 mm. Indeed in previous cervical rhizotomy experiments where good BDA labelling was obtained, the BDA injections were performed 10 days before termination and this timepoint may be optimal for labelling the injured sensory fibres.

Intrinsic Markers

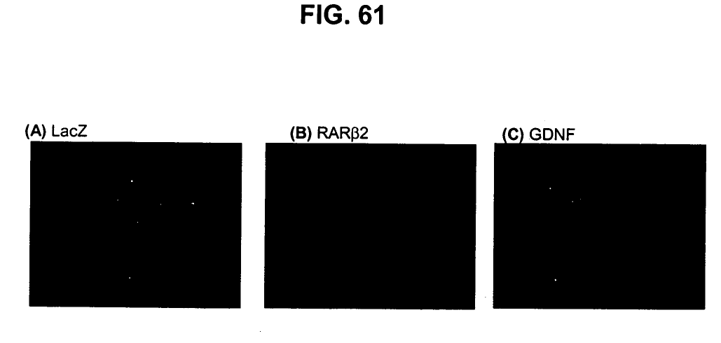

As a result of the lack of BDA labelling in the injured dorsal roots in the above experiments, we resorted to determining axonal regeneration in the treated/untreated groups using intrinsic markers. The neuropeptide calcitonin gene-related peptide (CGRP) identifies the small-diameter unmyelinated peptidergic class of DRG neurones and can be used to detect afferents into the spinal laminae I/II. There was increased CGRP staining in the RARβ2-treated animals in the superficial laminae in the spinal cord compared to the LacZ-treated animals (FIG. 61). This suggests that there was a higher amount of connectivity between the dorsal root axons and the spinal cord in the RARβ2 animals compared to the LacZ animals however this needs to be quantified. There was a slight increase in CGRP staining in GDNF animals however this again needs to be quantified.

pERK Labelling

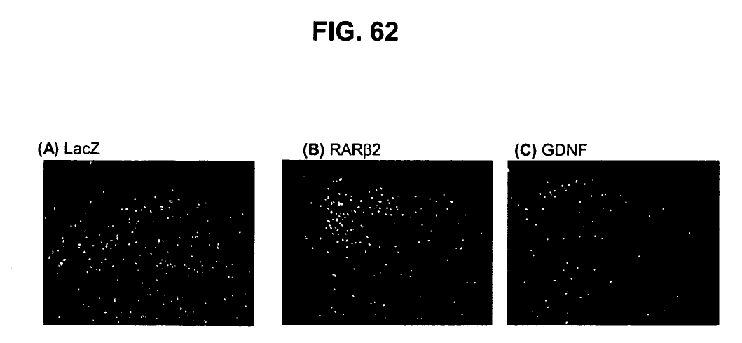

In the same study, prior to termination the injured forelimbs and the hindlimbs of the animals were dipped into a 56° C. waterbath to stimulate the primary sensory afferents. Thermal stimulation of the limbs will lead to an increase in pERK expression cells within the dorsal horn of the spinal cord, thus providing an additional test for the functional regeneration of primary sensory afferents and the establishment of re-connectivity in treated animals. pERK immunoreactivity could be detected in RARβ2 treated animals but could not be detected in LacZ treated animals (FIG. 62). A limited amount of pERK immunoreactivity was detected in the GDNF-treated animals. This again suggests that RARβ2 promoted functional regeneration of the sensory fibres in the dorsal root compared to control untreated groups. Thus this histological data correlated well with the behaviour data that was observed in the study.

Transduction Patterns in DRG Neurones

In the cervical rhizotomy model, the expression of the transgene is mediated by injection of EIAV vectors pseudotyped with rabies envelope into the spinal cord. Retrograde transport of the vector allowed the expression of the vectors in the cell bodies of the DRG neurones. In the DRG, the large-diameter neurones with Aβ fibres are responsible for mechanosensory function whilst the small-diameter neurones with C fibres account for nociception. The former can be identified by neurofilament staining, while the latter can be further divided into 2 subclasses that express either CGRP or can be identified by isolectin B₄ (1134) staining.

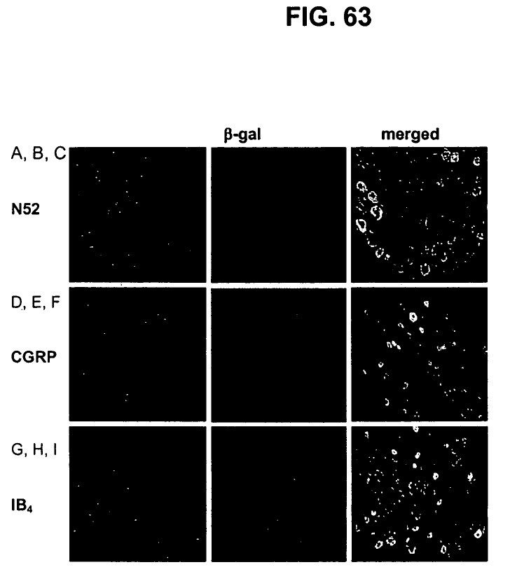

In order to distinguish the different subclasses of DRG neurones that are retrogradely transduced by the EIAV vectors, double immunohistochemistry was carried out with anti-β-galactosidase (β-gal) antibody in combination with either anti-neurofilament (N52), anti-CGRP or IB₄ antibodies. There was colocalisation of β-gal with N52, CGRP and IB₄, suggesting that both mechanoreceptive and nociceptive DRG neurones are transduced by EIAV vectors (FIG. 63).

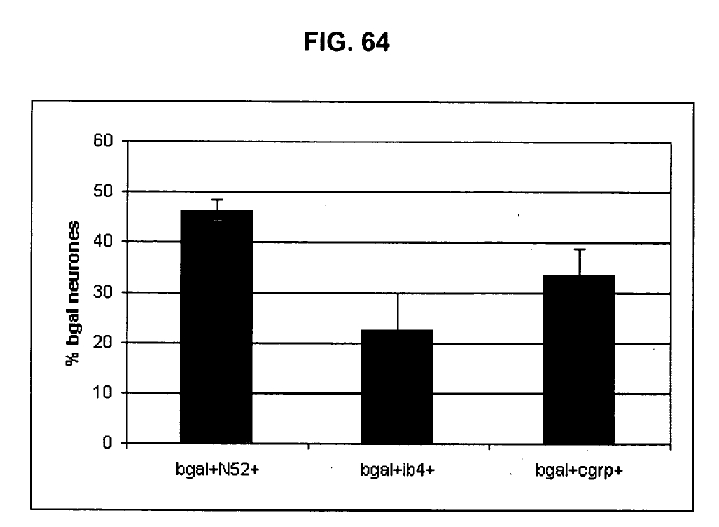

The number of doubly immunoreactive neurones was counted and expressed as a percentage of the transduced neurones. The data indicated that 46.2±2.2% of β-gal neurones were N52-positive, 33.6±5.1% of β-gal neurones were CGRP-positive and 22.5±7.7% of β-gal neurones were 11B₄-positive (FIG. 64). This suggests that all classes of DRG neurones were transduced.

2) Initiation of Further Experiment in Cervical Rhizotomy Model

A further experiment was initiated in order to increase the sample size of the animal groups used in cervical rhizotomy. In this second study animals were transduced with SMT2.Z or SMT.RARβ2 (n=4 in each group) 3 weeks before C₅-C₈ rhizotomy was performed. Baseline measurements for behavioural tests were obtained before rhizotomy. The rhizotomies have been performed and behavioural data is currently being collected.

3) Codon Optimised RARβ2

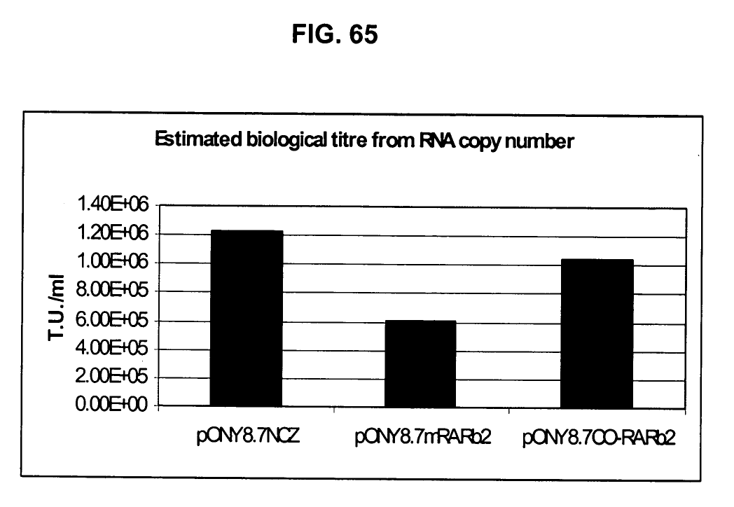

In order to further vector development in the Innurex™ programme, a codon optimised human RARβ2 was synthesised and cloned in the pONY8.7 and pONY8.95 series of vectors. This was then used to make unconcentrated EIAV preparations, after which the vectors were titred by evaluating packaging signal copy number in RNA extracted from the vector prep. The biological titre was estimated based on a comparison with a known titred bank. Results suggested that the codon optimised human RARβ2 was not detrimental to vector titres (at least by detecting RNA copy number) and increased the vector titre by 2-fold compared to wild type mouse RARβ2 (FIG. 65). The unconcentrated titres of the mouse wild type RARβ2 and the codon optimised RARβ2 vector preps were 6×10⁵ T.U./ml and 1×10⁶ T.U./ml respectively.

4) Mechanism of Action of RARβ2

The mechanism by which RARβ2 may promote regeneration of nerve fibres is being investigated.

One replicate of RNA from transduced and hemisectioned cord (7 day survival after injury) was analysed using Affymetrix chips (Neurobiology U34 arrays). When compared to the injured LacZ sample (LacZ 7d), the number of gene expression changes are listed below:

| | Upregulated ≧ 2 fold | Downregulated ≧ 2 fold |
|---|---|---|
| RARβ2 7d | 10 | 9 |
| GDNF 7d | 19 | 5 |

The low number of gene changes observed in this set of samples were surprising as in the previous report, it was determined that in uninjured cord samples 139 and 29 gene changes were observed in RARβ2 and GDNF transduced samples respectively (compared to LacZ). Further samples of transduced and hemisectioned cord (14 days survival after injury) were analysed on Neurobiology U34 arrays and the list of gene changes are listed below:

| | No. of genes | |
|---|---|---|
| | Replicates 2 and 3 | Replicates 1, 2 and 3 |
| Up 2 fold in RARB2 | 0 | 0 |
| Up 2 fold in GDNF | 4 | 2 |
| Down 2 fold in RARB2 | 6 | 5 |
| Down 2 fold in GDNF | 8 | 7 |

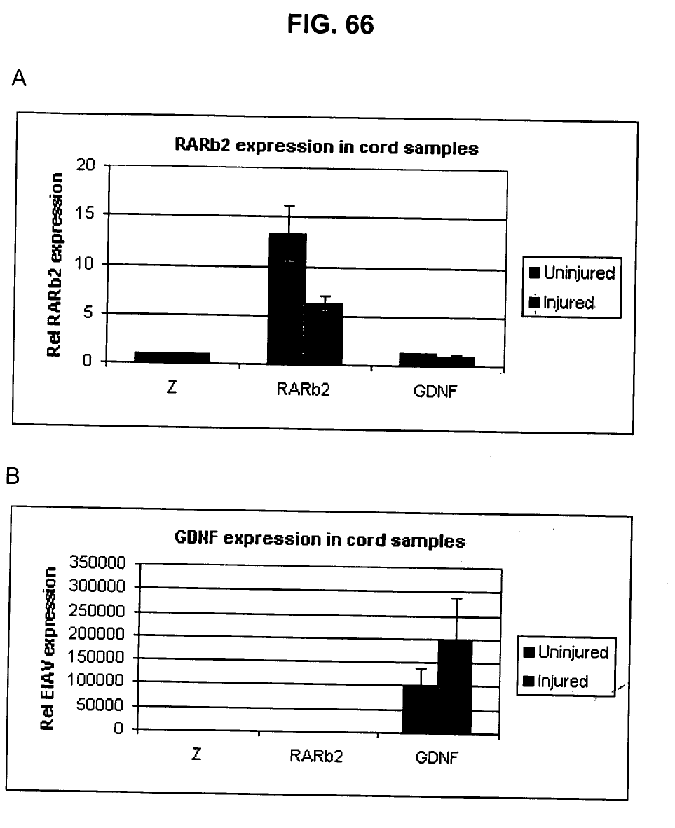

Again, the low number of gene changes in the injured samples was unexpected. Quantitative PCR analysis on the same samples was carried out to detect RARβ2 and GDNF expression in the samples to ascertain transduction efficiencies. At the same times, PCR was performed on previous uninjured samples. RARβ2 and GDNF expression could be detected in the respective tissues, and in both uninjured and injured tissue (FIG. 66). However the relative expression of RARβ2 in the injured spinal cord was half that of the uninjured sample. This decrease in RARβ2 expression in the injured spinal cord may be responsible for the low number of gene expression changes that were observed in the Affymetrix experiments. Alternatively, the low number of gene changes may be due to the delay in harvesting the tissue after injury (14 days).

Discussion

In this experiment immunohistochemistry was performed on tissue sections to determine if the improvement in sensorimotor task performance in RARβ2-treated animals could be used to correlate to anatomical evidence of axonal ingrowth from the injured dorsal roots into the spinal cord. At this time, we were unable to detect the anterograde tracer BDA that was used to label the regenerating axons in the samples however this may be due to a technical problem. Immunohistochemistry with instrinsic markers such as CGRP showed that there was increased CGRP immunoreactivity in RARβ2-treated animals compared to control animals, furthermore pERK immunoreactivity could be detected in RARβ2-treated animals and not in control LacZ animals. Together these data suggest that the functional correction in behaviour was correlated with restoration of connectivity at the molecular level. The experiments that have been initiated to increase the sample size of the treated and untreated groups in the above study, and the new data generated can be combined with the above to provide strong evidence for the in vivo regeneration capacities of RARβ2.

The recent data with transduced and injured rat spinal cords with the low number of gene expression changes was unexpected and the team has had to re-think the biology of spinal cord injury especially with regard to the timing of gene expression changes. In a review by Bareyre and Schwab (2003), it was indicated that a large number of gene changes occurred within the first 48 hours after spinal cord injury, and therefore this may be a more suitable timepoint to harvest the injured tissue. Small scale experiments are currently underway to investigate this issue.

Summary

The Examples demonstrate that RARfl2 and/or an agonist thereof can be used to cause neurite development.

In particular, we show inter alia the use of retinoids to stimulate neurite regeneration in peripheral nerves by activation of RARβ2.

The delivery and expression of nucleic acid sequences into non-dividing neuronal cells is demonstrated using retroviral vectors, in particular using EIAV pseudotyped with Rabies-G or VSV-G proteins.

Furthermore, we show that viral vectors can be produced for delivery of nucleic acid sequences encoding RARβ2 into cells.

Thus, neurite outgrowth and/or neurite regeneration are brought about via the vectors of the present invention delivering nucleic acid sequences encoding RARβ2 to non-dividing cells of the mammalian nervous system.

When peripheral nerves are damaged some regeneration can occur unlike nerves of the central nervous system which show no regeneration. However regeneration of peripheral nerves is limited particularly when there is traumatic nerve injury where there is a loss of nerve tissue such that a gap is created which the regenerating neurite cannot grow across. This delay in nerve regeneration can lead to muscle atrophy and lead to permanent disability.

In response to peripheral nerve injury neurotrophins are produced. These are a family of growth factors that are required for the survival of a variety of neurons. The family includes nerve growth factor (NGF) neurotrophin-3 (NT-3) and brain-derived neurotrophic factor (BDNF). It was hoped that neurotrophins could be used in the treatment of PNS injuries. However the results have not been encouraging. Two major problems have been encountered, firstly the problem of delivery to the injury, and secondly since different neurons need different neurotrophins a cocktail of them as to be administered in order for all the nerves to regenerate. We have investigated how neurotrophins stimulate neurite regeneration.

We have found that the vitamin A derivative all-trans-retinoic acid (tRA) like NGF induces neurite outgrowth from various embryonic sources, including PNS. Cellular effects of tRA are mediated by binding to nuclear receptors that are ligand activated transcription factors. There are two classes of receptors, retinoic acid receptors (RARs) and retinoid X receptors (RXRs), with three subtypes of each: α, β and γ. RAR receptors mediate gene expression by forming heterodimers with the RXRs, whereas RXRs can mediate gene expression either as homodimers or by forming heterodimers with orphan receptors.

We have found that only RARβ2 is required for neurite outgrowth of all types of neurons we have cultured. Furthermore when adult mouse DRG are cultured in the presence of NGF and an inhibitor of tRA synthesis, neurite outgrowth does not occur. Conversely, when tRA is added along with a blocking antibody to NGF, neurite outgrowth occurs as normal. We have also shown that NGF induces transcription of both the tRA-synthesizing enzyme RALDH-2 and the RARβ2 as well as a detectable release of synthesized tRA.

We propose that the stimulation of RARβ2 is an intrinsic requirement for the regeneration of neurites in the peripheral nervous system and that crucially this is downstream of the neurotrophins. Therefore in regard to the peripheral nervous system we want to administer retinoids that can activate the RARβ2 receptor in order for neurite regeneration to occur.

We have extended our observations to the CNS. We have found that the embryonic spinal cord expresses RARβ2 and that the amount of its expression correlates with the amount of neurite outgrowth. In contrast the adult spinal cord does not express RARβ2 nor can it regenerate neurites. We have shown that by transfecting RARβ2 by use of a defective herpes simplex virus type 1 (HSV-1) vector into cultured adult spinal cord we have transformed the normally inert spinal cord into one which can extend neurites. Therefore, we propose that gene therapy of injured spinal cord with RARβ2 will lead to functional recovery.

The use of retinoid to treat peripheral nervous system (PNS) injuries would have at least three major advantages over the use of neurotrophins. Firstly retinoids unlike neurotrophins are small lipophilic molecules which can be easily administered to the site of injury therefore regeneration should occur at a much quicker rate than can be achieved with neurotrophins, this should lead to a reduction in muscle atrophy and consequent paralysis. Secondly since the stimulation of RARβ2 is crucial to the regeneration of all neurons we have tested only one type of retinoid need be taken circumventing the need to administer a cocktail of neurotrophins. Thirdly retinoids are relatively easy to synthesise unlike neurotrophins.

Gene therapy with RARβ2 to treat CNS and/or PNS injuries should lead to functional recovery and therefore the prevention of paralysis.

Increasing RARβ2 levels according to the present invention preferably stimulates other neural repair mechanisms such as peripheral repair.

PNS and CNS injuries occur all over the world unfortunately it is unlikely that the incidence of such injuries will decrease. World wide a 1000 people per million of the population a year suffer spinal cord injury, ten times this number suffer some sort of PNS injury.

In addition there are three other areas where retinoids would be of use. In leprosy diabetes and AIDS neuropathy occurs (the neurites die) this is equivalent to PNS injury. In both leprosy and diabetes it has been shown that there is a loss of NGF in the skin of both types of patients leading to the loss of pain sensation and inflammation which can lead to ulcer formation. In AIDS patients sensory neuropathy is one of the most common effects of HIV infection, already NGF as been used to treat this condition.

Hence, we propose that RARβ2 agonists can be used to treat PNS injuries including neuropathy associated with leprosy, diabetes and AIDS. Gene therapy with RARβ2 can be used to treat CNS injuries and/or PNS injuries.

In summation, our results indicate a role for RA acting via RARβ2 in the outgrowth of neurites from certain classes of neurons.

The present invention therefore comprises a method of treatment of neurodegenerative disease in which expression of the retinoic acid receptor RARβ2 is ensured in affected cells or tissues. This may be achieved by treatment with an agonist of the RARβ2receptor and/or by gene therapy i.e. insertion of the nucleic acid coding for this receptor. The invention may also be seen as the use of these agents in medication for the treatment of peripheral nervous injuries and spinal cord regeneration e.g. in cases of paraplegia.

Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, biotechnology, chemistry or related fields are intended to be within the scope of the following claims. For example, it may be possible to substitute some or all of the RARβ2 and/or some or all of the RARβ2 agonist of the present invention with an inhibitor of an antagonist of RARβ2.

### REFERENCES TO RETINOIC ACID SECTION

1. Benbrook, D.; Lernhardt, E.; Pfahl, M.: A new retinoic acid receptor identified from a hepatocellular carcinoma. Nature 333: 669-672, 1988.

2. Brand, N.; Petkovich, M.; Krust, A.; Chambon, P.; de The, H.; Marchio, A.; Tiollais, P.; Dejean, A.: Identification of a second human retinoic acid receptor. Nature 332: 850-853, 1988.

3. Dejean, A.; Bougueleret, L.; Grzeschik, K.-H.; Tiollais, P.: Hepatitis B virus DNA integration in a sequence homologous to v-erb-A and steroid receptor genes in a hepatocellular carcinoma. Nature 322: 70-72, 1986.

4. de The, H.; del Mar Vivanco-Ruiz, M.; Tiollais, P.; Stunnenberg, H.; Dejean, A.:Identification of a retinoic acid responsive element in the retinoic acid receptor beta gene. Nature 343: 177-180, 1990.

5. de The, H.; Marchio, A.; Tiollais, P.; Dejean, A.: A novel steroid thyroid hormone receptor-related gene inappropriately expressed in human hepatocellular carcinoma. Nature 330: 667-670, 1987.

6. Kreczel, W.; Ghyselinck, N.; Samad, T. A.; Dupe, V.; Kastner, P.; Borrelli, E.; Chambon, P.: Impaired locomotion and dopamine signaling in retinoid receptor mutant mice. Science 279: 863-867, 1998.

7. Lotan, R.; Xu, X.-C.; Lippman, S. M.; Ro, J. Y.; Lee, J. S.; Lee, J. J.; Hong, W. K. Suppression of retinoic acid receptor-beta in premalignant oral lesions and its up-regulation by isotretinoin. New Eng. J. Med. 332: 1405-1410, 1995.

8. Mattei, M.-G.; de The, H.; Mattei, J.-F.; Marchio, A.; Tiollais, P.; Dejean, A.: Assignment of the human hap retinoic acid receptor RAR-beta gene to the p24 band of chromosome 3. Hum. Genet. 80: 189-190, 1988.

9. Mattei, M.-G.; Riviere, M.; Krust, A.; Ingvarsson, S.; Vennstrom, B.; Islam, M. Q.; Levan, G.; Kautner, P.; Zelent, A.; Chambon, P.; Szpirer, J.; Szpirer, C.: Chromosomal assignment of retinoic acid receptor (RAR) genes in the human, mouse, and rat genomes. Genomics 10: 1061-1069, 1991.

10. Nadeau, J. H.; Compton, J. G.; Giguere, V.; Rossant, J.; Varmuza, S.: Close linkage of retinoic acid receptor genes with homeobox- and keratin-encoding genes on paralogous segments of mouse chromosomes 11 and 15. Mammalian Genome 3: 202-208, 1992.

11. Samad, A.; Kreczel, W.; Chambon, P.; Borrelli, E.: Regulation of dopaminergic pathways by retinoids: activation of the D2 receptor promoter by members of the retinoic acid receptor-retinoid X receptor family. Proc. Nat. Acad. Sci. 94: 14349-14354, 1997.

### REFERENCES TO EXAMPLE 1

1. Lindsay, R. J. Neurosci. 8, 2394-2405 (1988).

2. Quinn, S. D. P. & De Boni, U. In vitro Cell. Dev. Biol. 27A, 55-62 (1991).

3. Haskell, B. E., Stach, R. W., Werrbach-Perez, K. & Perez-Polo, J. R. Cell Tissue Res. 247, 67-73 (1987).

4. Rodriguez-Tebar, A. & Rohrer, H. Development 112, 813-820 (1991).

5. Wion, D., Houlgatte, R., Barbot, N., Barrand, P., Dicou, E. & Brachet, P. Biochem. Biophys. Res. Comm. 149, 510-514 (1987).

6. Kastner, P., Chambon, P. & Leid, M. in Vitamin A in Health and Disease (ed. Blomhoff, R.) 189-238 (Dekker, New York, 1994).

7. Kliewer, S. A., Umesono, K., Evans, R. M. & Mangelsdorf, D. J. in Vitamin A in Health and Disease (ed. Blomhoff, R.) 239-255. (Dekker, New York, 1994)

8. Mangelsdorf, D. J. & Evans, R. M. Cell 83, 841-850 (1995).

9. Millbrandt, J. Neuron 1, 183-188 (1988).

10. McCaffery, P., Lee, M.-O., Wagner, M. A., Sladek, N. E. & Drager, U. Development 115, 371-382 (1992).

11. Duester, G. Biochemistry 35, 12221-12227 (1996).

12. Drager, U. C. & McCaffery, P. in Enzymology and Molecular Biology of Carbonyl Metabolism Vol. 5 (eds. Weiner, H. et al.) 185-192 (Plenum, New York, 1995).

13. Plum, L. A. & Clagett-Dame, M. Dev. Dynam. 205, 52-63 (1996).

14. Schnell, L., Schneider, R., Kolbeck, R., Barde, Y.-A. & Schwab, M. E. Nature 367, 170-173 (1994).

15. Schatzl, H. M. Trends Neurosci. 18, 463-464 (1995).

16. Maden, M., Sonneveld, E., van der Saag, P. T. & Gale, E. Development 125, 4133-4144 (1998).

### REFERENCES TO EXAMPLE 2

1. David, S. & Agayo, A. J. Axonal elongation into peripheral nervous system “bridges” after central nervous system injury in adult rats. Science 214, 931-933 (1981).

2. Cheng, H., Cao, Y. & Olsen, L. Spinal cord repair in adult paraplegic rats: partial restoration of hind limb function. Science 273, 510-513 (1996).

3. Schwab, M. E. Nerve fibre regeneration after traumatic lesions of the CNS; progress and problems. Phil. Trans. R. Soc. Lond. B 331, 303-306 (1991).

4. Bregman, B. S. et al. Recovery from spinal cord injury mediated by antibodies to neurite growth inhibitors. Nature 378, 498-501 (1995).

5. Schnell, L. et al. Neurotrophin-3 enhances sprouting of corticospinal tract during development and after adult spinal cord lesion. Nature 367, 170-173 (1994).

6. Li, Y. et al. Repair of adult rat corticospinal tract by transplants of olfactory ensheathing cells. Science 277, 2000-2002 (1997).

7. Kobayashi, N. R. et al. BDNF and NT4/5 prevent atrophy of rat rubrospinal neurons after cervical axotomy, stimulate GAP-43 and Tal-tubulin mRNA expression, and promote axonal regeneration. J. Neurosci. 17, 9583-9595 (1997).

8 Wagner, M. et al. Regional differences in retinoid release from embryonic neural tissue detected by an in vitro reporter assay. Development 116, 55-66 (1992).

9. Horton, C. & Maden, M. Endogenous distribution of retinoids during normal development and teratogenesis in the mouse embryo. Dev. Dynam. 202, 312-323 (1995).

10. McCaffery, P. & Drager, U. C. Hot spots of retinoic acid synthesis in the developing spinal cord. Proc. Natl. Acad. Sci. USA 91, 71947197 (1994).

11. McCaffery, P. & Drager, U. C. Retinoic acid synthesising enzymes in the embryopnic and adult vertebrate. In Enzymology and Molecular Biology of Carbonyl Metabolism 5 (H. Weiner et al. eds) pp173-183. Plenum Press, New York (1995).

12. Yamamoto, M. et al. Influence of the choroid plexus on cerebellar development:

analysis of retinoic acid synthesis. Dev. Brain Res. 93, 182-190 (1996).

13. Maden, M. et al. The distribution of endogenous retinoic acid in the chick embryo: implications for developmental mechanisms. Development 125, 4133-4144 (1998).

14. Maden, M. et al. Vitamin A-deficient quail embryos have half a hindbrain and other neural defects. Current Biol. 6, 417-426 (1996).

15. Maden, M. et al. The role of vitamin A in the development of the central nervous system. J. Nutr 128, 471S-475S (1998).

16. Maden, M. Retinoids in neural development. In Handbook of Experimental Pharmacology. (H. Nau & W. S. Blaner eds.) Springer-Verlag, Heidelberg (1998) in press.

17. Maden, M. et al. Retinoic acid as a chemotactic molecule in neuronal development. Int. J. Devl. Neurosci. 16, 317322 (1998).

18. Kastner, P et al. Role of nuclear retinoic acid receptors in the regulation of gene expression. In Vitamin A in Health and Disease. (R. Blomhoff ed.) ppl89-238. Marcel Dekker Inc., New York (1994).

19. Kliewer, S. A. et al. The retinoid X receptors: modulators of multiple hormonal signalling pathways. In Vitamin A in Health and Disease. (R. Blomhoff ed.) pp239-255. Marcel Dekker Inc., New York (1994).

20. Corcoran, J and Maden M. (1999). Nerve growth factor acts via retinoic acid synthesis to stimulate neurite outgrowth. Nat. Neuroscience 2, 307-308.

21. Quinn, S. D. P. & De Boni, U. Enhanced neuronal regeneration by retinoic acid of murine dorsal root ganglia and of fetal murine and human spinal cord in vitro. In vitro Cell. Dev. Biol. 27A, 55-62 (1991).

22. Wuarin, L. & Sidell, N. Differential susceptibilities of spinal cord neurons to retinoic acid-induced survuval and differentiation. Dev. Biol. 144, 429-435 (1991).

23. Ved, H. S. & Pieringer, R. A. Regulation of neuroanl differentiation by retinoic acid alone and in cooperation with thyroid hormone or hydrocortisone. Dev. Neurosci. 15, 49-53 (1993).

24. Corcoran, J. & Maden, M. Nerve growth factor acts via retinoic acid synthesis to stimulate neurite outgrowth. Nature Neurosci. in press (1999).

25. Caroni, P. & Schwab, M. E. Codistribution of neurite growth inhibitors and oligodendrocytes in rat CNS: appearance follows nerve fiber growth and precedes myelination. Dev. Biol. 136, 287-295. (1989).

26. Notterpek, L. M. & Rome, L. H. Functional evidence for the role of axolemma in CNS myelination. Neuron 13, 473-485. (1994).

27. Smith, D. S. & Skene, J. H. P. A transcription-dependent switch controls competence of adult neurons for distinct modes of axon growth. J Neurosci. 17, 646-658 (1997).

28. Lim, F., Hartley, D., Starr, P., Song, S., Lang, P., Yu, L., Wang, Y. M. & Geller, A. I. Use of defective herpes-derived plasmid vectors. Meth. Mol. Biol. 62, 223-232 (1997).

### REFERENCES TO EXAMPLE 3

Achkar, C. C., Dergiuni, F., Blumberg, B., Langston, A., Levin, A. A., Speck, J., Evans, R. M., Bolado, J., Nakanishi, K., Buck, J. and Gudas, L. J. (1996). 4-oxoretinol, a new natural ligand and transactivator of the retinoic acid receptors. Proc. Natl. Acad. Sci. USA 93, 4879-4884.

Ang, H. L. and Duester, G. (1997). Initiation of retinoid signalling in primitive streak mouse embryos: spatiotemporal expression patterns of receptors and metabolic enzymes for ligand synthesis. Dev. Dynam. 208, 536-543.

Barde, Y.-A., Edgar, D. and Thoenen, H. (1982). Purification of a new neurotrophic factor from mammalian brain. EMBO J. 1, 549-553.

Buck, J., Derguini, F., Levi, E., Nakanishi, K. and Hammerling, U. (1991). Intracellular signalling by 14-hydroxy-4,14-retro-retinol. Science 254, 1654-1656.

Campenot, R. B. (1977). Local control of neurite development by nerve growth factor. Proc. Natl. Acad. Sci. USA 74, 4516-4519.

Crowley, C., Spencer, S. D., Nishimura, M. C., Chen, K. S., Pitts-Meek, S., Armanini, M. P., Ling, L. H., McMahon, S. B., Shelton, D. L, Levinson, A. D. and Phillips, H. S. (1994). Mice lacking nerve growth factor display perinatal loss of sensory and sympathetic neurons yet develop basal forebrain cholinergic neurons. Cell 76, 1001-1012.

Corcoran, J and Maden M. (1999). Nerve growth factor acts via retinoic acid synthesis to stimulate neurite outgrowth. Nat. Neuroscience 2, 307-308.

Drager, U. C. and McCafery, P. (1995). Retinoic acid synthesis in the developing spinal cord. In Enzymology and Molecular Biology of Carbonyl Metabolism. 5 (ed. H. Weiner et al.) 185-192 (Plenum Press, New York.

Emfors, P, Lee, K, Kucera. J. and Jaenisch, R. (1994). Lack of Neurotrophin-3 leads to deficiencies in the peripheral nervous system and loss of limb proprioceptive afferents. Cell 77, 503-512.

Farinas, I., Jones, K. R., Backus, C., Wang, X-Y. and Reichardt, L. F. (1994). Severe sensory and sympathetic deficits in mice lacking neurotrophin-3. Nature 369, 658-661.

Godbout, R., Packer, M., Poppema, S. & Dabbath, L. (1996). Localization of cytosolic aldehyde dehydrogenase in the developing chick retina: in situ hydridisation and immunohistochemical analyses. Dev. Dynam. 205, 319-331.

Jones, K. R, Farlinas, I, Backus, C. and Reichardt, L. F. (1994). Targeted disruption of the BDNF gene perturbs brain and sensory neuron development but not motor neuron development. Cell 76, 989-999.

Klein, R., Silos-Santiago, I., Smeyne, R. J., Lira, S. A., Brambilla, R., Bryant, S., Zhang, L., Snider, W. D. and Barbacid. M. (1994). Disruption of the neurotrophin-3 receptor gene trkC eliminates 1a muscle afferents and results in abnormal movements. Nature 368, 249-251.

Klein, R., Smeyne, R. J., Wurst, W., Long, L. K., Auerbach, B. A., Joyner, A. L. and Barbacid. M. (1993). Targeted disruption of the trkb neurotrophin receptor gene results in nervous system lesions and neonatal death. Cell 75, 113-122.

Kastner, P., Chambon, P. and Leid, M. (1994). Role of nuclear retinoic acid receptors in the regulation of gene expression. In Vitamin A in Health and Disease. (R. Blomhoff ed.) pp 189-238. Marcel Dekker Inc., New York.

Kliewer, S. A., Umesono, K., Evans, R. M. and Mangelsdorf, D. J. (1994). The retinoid X receptors: modulators of multiple hormonal signalling pathways. In Vitamin A in Health and Disease. (R. Blomhoff ed.) pp 239-255. Marcel Dekker Inc., New York.

Leid, M., Kastner, P. and Chambon, P. (1992). Multiplicity generates diversity in the retinoic signalling pathways. Trends Biol. Sci. 17, 427-433

Levi-Montanlcini, R. The nerve growth factor: Thirty five years later. Science 237, 1154-1164 (1987).

Lindsay, R. (1998). Nerve growth factors (NGF, BDNF) enhance axonal regeneration but are not required for survival of adult sensory neurons. J. Neurosci. 8, 2394-2405.

Maden, M., Gale, E., Kostetskii, I. and Zile, M. (1996). Vitamin A-deficient quail embryos have half a hindbrain and other neural defects. Current Biol. 6, 417-426.

Maden, M. Retinoids in neural development. In Handbook of Experimental Pharmacology. (H. Nau & W. S. Blaner eds.) Springer-Verlag, Heidelberg (1998) in press.

Maden, M., Gale, E. and Zile, E. (1998). The role of vitamin A in the development of the central nervous system. J. Nutr. 128, 471S-475S.

Maden, M., Sonneveld, E., van der Saag, P. T. and Gale, E. (1998). The distribution of endogenous retinoic acid in the chick embryo: implications for developmental mechanisms. Development 125 in press.

Mangelsdorf, D. J. and Evans, R. M. (1995). The RXR heterodimers and orphan receptors. Cell 83, 841-850.

Maisonpierre. P. C., Belluscio, L., Squinto, S., Ip, N. Y., Furth, M. E., Lindsay, R. M. and Yancopoulos, G. D. (1990). Neurotrophin-3: a neurotrophic factor related to NGF and BDNF. Science 247, 1446-1451.

McCaffery, P. and Drager, U. C. (1994). Hot spots of retinoic acid synthesis in the developing spinal cord. Proc. Natl. Acad. Sci. USA 91, 7194-7197.

McCaffery, P., Lee, M.-O., Wagner, M. A., Sladek, N. E. & Drager, U. (1992). Asymmetrical retinoic acid synthesis in the dorsoventral axis of the retina. Development 115, 371-382.

McCormick, A. M., Napoli, J. L., Schnoes, H. K. & Deluca, H. F. (1978). Isolation and identification of 5,6-epoxyretinoic acid: a biologically active metabolite of retinoic acid. Biochemistry 17, 4084-4090.

Millbrandt, J. (1989). Nerve growth factor induces a gene homologous to the glucocorticoid receptor gene. Neuron 1, 183-188.

Niederreither, K., McCaffery, P., Drager, U. C., Chambon, P. and Dolle, P. (1997). Restricted expression and retinoic acid-induced downregulation of the retinaldehyde dehydrogenase type 2 (RALDH-2) gene during mouse development. Mech of dev 62, 67-68 Quinn, S. D. P and De Boni, U. (1991). Enhanced neuronal regeneration by retinoic acid of murine dorsal root ganglia and of fetal murine and human spinal cord in vitro. In vitro Cell. Dev. Biol. 27A, 55-62.

Smeyne, R. J., Klein, R., Schnapp, A., Long, L. K., Bryant, S., Lewin, A, Lira, S. A. and Barbacid, M. (1994). Severe sensory and sympathetic neuropathies in mice carrying a disrupted trk/NGF receptor gene. Nature 368, 246-249.

Snider, W. D. (1994). Functions of the neurotrophins during nervous system development what the knockouts are teaching us. Cell 77, 627-638.

Tuttle R. & Mathew, W. D (1995). Neurotrophins affect the pattern of DRG neurite growth in a bioassay that presents a choice of CNS and PNS substrates. Development 121, 1301-1309.

Wuarin, L. & Sidell, N. (1991). Differential susceptibilities of spinal cord neurons to retinoic acid-induced survival and differentiation. Dev. Biol. 144, 429-435

### REFERENCES TO EXAMPLE 4

Lim, F., Hartley, D., Starr, P., Song, S., Lang, P., Yu, L., Wang, Y. M. & Geller, A. I. Use of defective herpes-derived plasmid vectors. Meth. Mol. Biol. 62, 223-232 (1997).

World Intellectual Property Organization publication number WO 98/17817 (IMPROVED RETROVIRAL VECTORS).

World Intellectual Property Organization publication number WO 98/17816 LENTIVIRAL VECTORS (Tradsducing non-dividing cells).

World Intellectual Property Organization publication number WO 99/61639 DELIVERY SYSTEM (Pseudotyping with Rabies G).

We claim:

1. A method for treating nerve injury in a mammal in need of such treatment, comprising administering a vector to a target cell in injured nervous tissue of the mammal, said vector comprising a nucleic acid sequence encoding retinoic acid receptor β2 (RARβ2) operably linked to a promoter, wherein RAR β2 is expressed in the target cell thereby promoting growth of said injured nervous tissue.

2. The method of claim 1, wherein said vector is a viral vector.

3. The method of claim 2, wherein said viral vector is a lentiviral vector.

4. The method of claim 3, wherein said lentiviral vector is selected from the group consisting of an HIV (Human Immunodeficiency Virus) lentiviral vector, an EIAV (Equine Infectious Anemia Virus) lentiviral vector, and an FIV (Feline Immunodeficiency Virus) lentiviral vector.

5. The method of claim 3, wherein said lentiviral vector is pseudotyped with a heterologous envelope.

6. The method of claim 5, wherein said heterologous envelope is VSV-G or Rabies-G.

7. The method of claim 3, wherein said lentiviral vector is a minimal lentiviral vector.

8. The method of claim 1, wherein said nerve injury is a spinal cord injury or a peripheral nerve injury.

9. The method of claim 1, wherein said nerve injury is an avulsion injury.

10. The method of claim 1, wherein the treatment of the nerve injury results in at least one therapeutic effect relating to said nerve injury, said at least one therapeutic effect selected from the group consisting of regeneration of neurons or nerve fibers, promotion of neurite outgrowth, reduction of inflammation, reduction of apoptotic cell death, promotion of sensory recovery, and promotion of locomotor function recovery.

11. The method of claim 10, wherein said regeneration of neurons or nerve fibers comprises the regeneration of axons.

12. The method of claim 11, wherein said regeneration of axons comprises increased functional connectivity of axons in spinal cord.

13. The method of claim 1, wherein said promoter is a constitutive promoter, an inducible promoter or a tissue-specific promoter.

14. The method of claim 1, wherein administration of said vector is into or near site of nerve injury or into spinal cord.

15. The method of claim 1, wherein the nucleic acid sequence encoding RARβ2 is codon optimized.

16. A pharmaceutical composition comprising a minimal lentiviral vector comprising a nucleic acid sequence encoding RARβ2 in operable linkage with a promoter, and a pharmaceutically acceptable carrier or diluent.

17. The pharmaceutical composition of claim 16, wherein said nucleic acid sequence encoding RARβ2 is codon optimized.

18. The pharmaceutical composition of claim 16, wherein said minimal lentiviral vector is an HIV, EIAV, or FIV lentiviral vector.

19. The pharmaceutical composition of claim 16, wherein said promoter is a constitutive promoter, an inducible promoter or a tissue-specific promoter.

20. The pharmaceutical composition of claim 16, wherein said lentiviral vector is pseudotyped with a heterologous envelope.

21. The pharmaceutical composition of claim 20, wherein said heterologous envelope is VSV-G or Rabies-G.

22. A method for expressing RARβ2 in a target cell in nervous tissue, comprising contacting the target cell with a vector, said vector comprising a nucleic acid sequence encoding retinoic acid receptor β2 (RARβ2) operably linked to a promoter, wherein RARβ2 is expressed in the target cell thereby promoting growth of said nervous tissue.

23. The method of claim 22, wherein said vector is a viral vector.

24. The method of claim 23, wherein said viral vector is a lentiviral vector.

25. The method of claim 24, wherein said lentiviral vector is selected from the group consisting of an HIV (Human Immunodeficiency Virus) lentiviral vector, an EIAV (Equine Infectious Anemia Virus) lentiviral vector, and an FIV (Feline Immunodeficiency Virus) lentiviral vector.

26. The method of claim 24, wherein said lentiviral vector is pseudotyped with a heterologous envelope.

27. The method of claim 26, wherein said heterologous envelope is VSV-G or Rabies-G.

28. The method of claim 24, wherein said lentiviral vector is a minimal lentiviral vector.

29. The method of claim 22, wherein said nervous tissue is injured nervous tissue.

30. The method of claim 22, wherein growth of said nervous tissue comprises regeneration of neurons or nerve fibers, or promotion of neurite outgrowth.

31. The method of claim 30, wherein said regeneration of neurons or nerve fibers comprises the regeneration of axons.

32. The method of claim 22, wherein said promoter is a constitutive promoter, an inducible promoter or a tissue-specific promoter.

33. The method of claim 22, wherein the nucleic acid sequence encoding RARβ2 is codon optimized.
